(19) United States
(12) Patent Application Publication
(10) Pub. No.: US 2024/0132910 A1
(43) Pub. Date: Apr. 25, 2024

(54) USE OF HISTIDINE RICH PEPTIDES AS A TRANSFECTION REAGENT FOR rAAV AND rBV PRODUCTION

(71) Applicant: BioMarin Pharmaceutical Inc., Novato, CA (US)

(72) Inventors: Irina PEREVOSHCHIKOVA, Novato, CA (US); Baran ARSLAN, Novato, CA (US); Julie GARDIN, Novato, CA (US); Pauline BOURBON, Novato, CA (US); Helai ARABSHAHI, Novato, CA (US); Tomas CINEK, Novato, CA (US); Shilpa SHROFF, Berkeley, CA (US)

(73) Assignee: BioMarin Pharmaceutical Inc., Novato, CA (US)

(21) Appl. No.: 18/277,141

(22) PCT Filed: Feb. 10, 2022
(86) PCT No.: PCT/US2022/015903

§ 371 Date: Aug. 14, 2023

Related U.S. Application Data
(60) Provisional application No. 63/149,425, filed on Feb. 15, 2021.

(51) Int. Cl. C12N 15/86; C12N 5/07; C12N 5/073

(52) U.S. Cl. CPC C12N 15/86; C12N 5/0601; C12N 5/0603; C12N 2710/14051; C12N 2750/14151

(57) ABSTRACT

The present invention provides methods, compositions, and kits for preparing and using adeno associated virus and baculovirus. The methods for producing adeno associated virus and baculovirus particles include using histidine rich peptides and other cationic peptides as transfection reagents. The adeno associated virus are pseudotyped with capsids, in particular for use in gene therapy and/or diagnostics. The baculovirus are also used to prepare adeno associated virus.

### FIELD OF THE INVENTION

The present invention is directed to methods and processes for producing recombinant adeno-associated virus (rAAV) and recombinant baculovirus (rBV) particles using histidine rich peptides (HRPs) as a transfection reagent.

### BACKGROUND OF THE INVENTION

The present invention is directed to methods of improving adeno associated virus (AAV) production. AAV are non-enveloped viruses with single-stranded DNA genome of 20-25 nm (4.7 kb). AAV has a linear single-stranded DNA (ssDNA) genome of approximately 4.7-kilobases (kb), with two 145 nucleotide-long inverted terminal repeats (ITR) at the termini. The virus does not encode a polymerase and therefore relies on cellular polymerases for genome replication. The ITRs flank the two viral genes—rep (replication) and cap (capsid), encoding non-structural and structural proteins, respectively. The rep gene, through the use of two promoters and alternative splicing, encodes four regulatory proteins that are dubbed Rep78, Rep68, Rep52, and Rep40. These proteins are involved in AAV genome replication and packaging. The cap gene, through alternative splicing and initiation of translation, gives rise to three capsid proteins, VP1 (virion protein 1), VP2 and VP3, with molecular weight of 87, 72, and 62 kDa, respectively. These capsid proteins assemble into a near-spherical protein shell of 60 subunits.

AAV are unable to replicate on their own and require co-infection with a helper virus, typically adenovirus or herpesvirus. When AAV infects a human cell alone, its gene expression program is auto-repressed and latent infection of the cell occurs. However, when a latently infected cell is co-infected with a helper virus, such adenovirus or herpes simplex virus, AAV gene expression is activated leading to excision of the provirus DNA from the host cell chromosome, followed by replication and packaging of the viral genome.

Production of viral vectors in mammalian or insect cells requires the delivery of foreign nucleic acids coding the gene of interest and necessary proteins involved in viral vector production. Passive uptake of foreign nucleic acids into the cells is restricted to protect the cells from possible infections. Facilitated delivery of nucleic acid is therefore required. Most commonly a transfection reagent is used to shuttle the nucleic acids into the cells. The choice of the transfection reagent for large scale manufacturing of viral vectors is governed by its efficiency, safety, consistency, scalability and cost.

A suitable transfection reagent should meet several criteria to be a successful candidate for large scale viral vector production: 1) preferably bind the foreign nucleic acids provided in the forms of plasmids, bacmids or any other forms; 2) complexes of foreign DNA and the transfection reagent is preferably stable for more than 10 minutes; 3) complexes of foreign DNA: transfection reagent bind to the cell surface and be taken up by the cells; 4) the transfection reagent preferably provides a way for endosomal release of the foreign DNA or foreign DNA:transfection reagent complexes; 5) foreign DNA or foreign DNA: transfection reagent complexes is preferably able to reach the nucleus of the cell; 6) foreign DNA or foreign DNA:transfection reagent complexes is preferably available for transcription of the proteins and replication of the genes; 7) the transfection reagent and foreign DNA:transfection reagent complexes is preferably well tolerated by the cells; 8) foreign DNA or foreign DNA:transfection reagent complexes is preferably compatible with the production media of choice; and 9) the transfection reagent is preferably able to transfect the production cells efficiently.

Currently commercially available transfection reagents lack one or more of the criteria mentioned above. For example, PEIPRO® (G1VIP grade, PolyPlus), a widely used transfection reagent of choice for the production of viral vectors and vaccine that includes linear polyethylenimine(s) (PEI), is known for the poor stability of DNA:PEI complexes (Sang Y. et al. Salt ions and related parameters affect PEI-DNA particle size and transfection efficiency in Chinese hamster ovary cells Cytotechnology 2015, 67(1):67-74; Han X. et al., The heterogeneous nature of polyethylenimine-DNA complex formation affects transient gene expression Cytotechnology 2009, 60(1-3):63).

For different productions systems, the transfection reagent directly affects the scaling the productions of biologic materials. To this end, transfection reagents such PEI are incapable of expanding the size of the productions. For example, it is noted that human embryonic kidney 293 (HEK293) cell culture productions are limited to volumes of 500 Liters (L) or less.

In addition, lipid based transfection reagents are unstable in the diluted form before complexation with DNA. Also, transfection reagents used for insect and mammalian cells (e.g., CELLFECTIN®, TRANSIT®, and FECTOVIR®) are not yet available in Good Manufacturing Practice (GMP) grade, which is desirable for rAAV production to be used for clinical purposes.

Therefore, there is a need for a better transfection reagent suitable for large scale manufacturing of viral vectors in a GMP setting.

### SUMMARY OF THE INVENTION

In various embodiments, methods for preparing recombinant adeno-associated virus (rAAV) and recombinant baculovirus (rBV) are disclosed. Also in various embodiments, methods for generating cells stably expressing elements for producing rAAV are disclosed. The methods include the use of cationic peptides as transfection reagents, where the peptides have a positive charge at a pH ranging from 6 to 8 (e.g., 7.4). Some exemplary cationic peptides include histidine rich peptides (HRPs) are capable of electrostatically interacting with deoxyribonucleic acid (DNA) and penetrating cell membranes such that the DNA is delivered into the cell. Thus, HRPs can be use as transfection reagents for delivering plasmids and bacmids to cells.

The rAAV production methods of various embodiments include the steps of co-transfecting cells with one or more vectors for rAAV production using a cationic peptide such as an HRP, culturing the transfected cells to generate rAAV, and optionally, recovering the rAAV.

In other embodiments, the rAAV production methods include the steps of transiently co-transfecting cells suspended in a culture volume of more than 500 liters (L) with one or more vectors for rAAV production using a transfection reagent, culturing the transfected cells to generate rAAV, and optionally, recovering the rAAV.

The rBV production methods of various embodiments include the steps of transfecting cells with recombinant bacmids having a heterologous nucleotide sequence using a cationic peptide such as an HRP, culturing the transfected cells to generate rBV and optionally, recovering the rBV.

In other embodiments, the rBV production methods include the steps of transfecting cells with a heterologous nucleotide sequence using a cationic peptide such as an HRP, culturing the transfected cells to generate rBV, and optionally, recovering the rBV. The cells have at least a portion of a baculovirus genome. During the culturing step, the heterologous nucleotide sequence and the at least a portion of a baculovirus genome combine to form a baculovirus genome capable of generating rBV.

In other embodiments, the rBV production methods include the steps of co-transfecting cells with a complete or partial baculovirus genome, either circular or linearized, and with a heterologous nucleotide sequence (which can be incorporated in a transfer vector) containing a region or regions homologous to the baculovirus genome, using a cationic peptide such as an HRP, culturing the transfected cells to generate rBV, and optionally, recovering the rBV. During the culturing step, the complete or partial baculovirus genome and the heterologous nucleotide sequence recombine via the homologous regions to form a recombinant baculovirus genome capable of generating rBV and carrying at least a portion of the heterologous nucleotide sequence.

The rBV production methods of various embodiments include the steps of transfecting cells with recombinant bacmids having a nucleotide sequence providing an rAAV genome vector or encoding Rep or Cap proteins using a cationic peptide such as an HRP, culturing the transfected cells to generate rBV, and optionally, recovering the rBV.

In other embodiments, the rBV production methods include the steps of transfecting cells with a nucleotide sequence providing an AAV genome vector or encoding Rep or Cap proteins using a cationic peptide such as an HRP, culturing the transfected cells to generate rBV, and optionally, recovering the rBV. The cells have at least a portion of a baculovirus genome. During the culturing step, the nucleotide sequence and the at least a portion of a baculovirus genome combine to form a baculovirus genome capable of generating rBV.

In other embodiments, the rBV production methods include the steps of co-transfecting cells with a complete or partial baculovirus genome, either circular or linearized, and with a heterologous nucleotide sequence (which can be incorporated in a transfer vector) containing either a rAAV vector genome or AAV rep and/or cap genes, and also containing a region or regions homologous to the baculovirus genome, using a cationic peptide such as an HRP, culturing the transfected cells to generate rBV, and optionally, recovering the rBV. During the culturing step, the complete or partial baculovirus genome and the heterologous nucleotide sequence recombine via the homologous regions to form a recombinant baculovirus genome capable of generating rBV, carrying at least a portion of the heterologous nucleotide sequence.

The cell generating methods of various embodiments include the steps of transfecting cells with a vector with a nucleotide sequence encoding an element used for generating rAAV using a cationic peptide such as an HRP and isolating a transfected cell having a genome with the nucleotide sequence.

In various embodiments, the cationic peptide of any embodiment including an amino acid sequence that is at least 85% identical to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124.

In various embodiments, a cationic peptide is disclosed having an amino acid sequence that is at least 85% identical to any one of SEQ ID NO: 93, 94, 95, 96, 97, 98, 99, 100, 101, 102, 103, 104, 105, 106, 107, 108, 121, 122, 123, or 124. In other embodiments, a cationic peptide is disclosed having an amino acid sequence of SEQ ID NO: 93, 94, 95, 96, 97, 98, 99, 100, 101, 102, 103, 104, 105, 106, 107, 108, 121, 122, 123, or 124.

In various embodiments, a composition is disclosed that include one or more cationic peptides having an amino acid sequence that is at least 85% identical to any one of SEQ ID NO: 93, 94, 95, 96, 97, 98, 99, 100, 101, 102, 103, 104, 105, 106, 107, 108, 121, 122, 123, 124, or combinations thereof. In other embodiments, compositions are disclosed that include one or more cationic peptides having an amino acid sequence of SEQ ID NO: 93, 94, 95, 96, 97, 98, 99, 100, 101, 102, 103, 104, 105, 106, 107, 108, 121, 122, 123, 124, or combination thereof.

### BRIEF DESCRIPTION OF THE DRAWINGS

FIG. 1 discloses titers of Bba41 capsids produced in human embryonic kidney 293 (HEK293) cells cultured in Ambr15 minibioreactors (15 milliliter (mL) volume) and transfection efficiencies of different histidine rich peptides (HRPs) and PEIPRO® at 24 and 66 hours post transfection. The cells were transfected with plasmids for producing Bba41 capsids and providing an AAV genome vector containing a green fluorescence protein (GFP) expression cassette. The transfection efficiency was measured using flow cytometry to identify cells expressing GFP protein. The AAV titers were quantified using digital droplet polymerase chain reaction (ddPCR).

FIG. 2 shows the transfection efficiency of the LAH4 peptide in HEK293 cells at 24 hours post transfection in shake flasks (30 mL working volume) when transfecting plasmids for producing Bba41 capsids and providing an AAV genome vector containing a GFP expression cassette. The transfection efficiency was measured using flow cytometry to identify cells expressing GFP protein.

FIG. 3 shows the GFP fluorescence intensity of HEK293 cells at 24 hours post transfection in shake flasks (30 mL working volume) transfected with plasmids for producing Bba41 capsids and providing an AAV genome vector containing an GFP expression cassette using different LAH4 peptide concentrations and complexation volumes. The transfection efficiency was measured using flow cytometry to identify cells expressing GFP protein.

FIG. 4 shows the titers of Bba41 capsids produced in HEK293 cells at 72 hours post transfection in shake flasks (30 mL working volume) transfected with plasmids for producing Bba41 capsids and providing an AAV genome vector containing an GFP expression cassette using different LAH4 peptide concentrations and complexation volumes. The AAV titers were quantified using ddPCR.

FIGS. 5A, 5B, and 5C show the cell densities of HEK293 cells in shake flasks (30 mL working volume) transfected with plasmids for producing Bba41 capsids and providing an AAV genome vector containing an GFP expression cassette using different LAH4 peptide concentrations and complexation volumes. The cell densities were assessed at 0 hours, 24 hours, 48 hours, and 72 hours post transfection. The cell densities were quantified using an automated cell counter and Trypan Blue exclusion.

FIGS. 6A, 6B, and 6C show the viabilities of HEK293 cells in shake flasks (30 mL working volume) transfected with plasmids for producing Bba41 capsids and providing an AAV genome vector containing an GFP expression cassette using different LAH4 peptide concentrations and complexation volumes. The viabilities were assessed at 0 hours, 24 hours, 48 hours, and 72 hours post transfection. The cell viabilities were quantified using an automated cell counter and Trypan Blue exclusion.

FIG. 7 discloses titers of Bba41 capsids produced in HEK293 cells cultured in shake flasks (30 mL working volume) and transfected with plasmids for producing Bba41 capsids and providing an AAV genome vector containing an GFP expression cassette using different HRPs and PEIPRO®. The different HRPs and PEIPRO® were mixed with the plasmids and incubated for different times prior to transfection. The AAV titers were quantified using ddPCR.

FIG. 8A shows the transfection efficiency of different HRPs and PEIPRO® in HEK293 cells cultured in shake flasks (30 mL working volume) at 24 hours post transfection when transfecting plasmids for producing Bba41 capsids and providing an AAV genome vector containing an GFP expression cassette. The different HRPs and PEIPRO® were mixed with the plasmids and incubated for different times prior to transfection. The transfection efficiency was measured using flow cytometry to identify cells expressing GFP protein.

FIG. 8B shows the transfection efficiency of different HRPs in HEK293 cells cultured in shake flasks (30 mL working volume) at 48 hours post transfection when transfecting plasmids for producing Bba41 capsids and providing an AAV genome vector containing an GFP expression cassette. The different HRPs were mixed with the plasmids and incubated for different times prior to transfection. The transfection efficiency was measured using flow cytometry to identify cells expressing GFP protein.

FIG. 9 shows the titers of AAV9 capsids produced in HEK293 cells in 1.6 L shake flasks (500 mL working volume) transfected with plasmids for producing AAV9 capsids and providing an AAV genome vector containing a gene of interest using the LAH4 peptide and PEIPRO®. The AAV titers were quantified using ddPCR.

FIG. 10A shows the transfection efficiency of different complexation volumes of the LAH4 peptide in HEK293 cells in Ambr15 minibioreactors (15 mL volume) at 48 hours post transfection when transfecting plasmids for producing AAV9 capsids and providing an AAV genome vector containing an GFP expression cassette. The transfection efficiency was measured using flow cytometry to identify cells expressing GFP protein.

FIG. 10B shows the transfection efficiency of different complexation volumes of the LAH4 peptide in HEK293 cells in Ambr15 minibioreactors (15 mL volume) at 24 hours post transfection when transfecting plasmids for producing AAV9 capsids and providing an AAV genome vector containing an GFP expression cassette. The transfection efficiency was measured using flow cytometry to identify cells expressing GFP protein.

FIG. 10C shows the titers of AAV9 capsids produced in HEK293 cells in in Ambr15 minibioreactors (15 mL volume) and transfected with plasmids for producing AAV9 capsids and providing an AAV genome vector containing an GFP expression cassette using different complexation volumes of the LAH4 peptide. The AAV titers were quantified using ddPCR.

FIG. 10D shows the viability of HEK293 cells in Ambr15 minibioreactors (15 mL volume) and transfected with plasmids for producing AAV9 capsids and providing an AAV genome vector containing an GFP expression cassette using different complexation volumes of the LAH4 peptide. The cell viabilities were quantified using an automated cell counter and Trypan Blue exclusion.

FIG. 10E shows the transfection efficiency of the LAH4 peptide in HEK293 cells in Ambr15 minibioreactors (15 mL volume) at 48 hours post transfection when transfecting plasmids for producing AAV9 capsids and providing an AAV genome vector containing an GFP expression cassette, where the LAH4 peptide and plasmids were mixed and incubated for different times prior to transfection. The transfection efficiency was measured using flow cytometry to identify cells expressing GFP protein.

FIG. 10F shows the transfection efficiency of the LAH4 peptide in HEK293 cells in Ambr15 minibioreactors (15 mL volume) at 24 hours post transfection when transfecting plasmids for producing AAV9 capsids and providing an AAV genome vector containing an GFP expression cassette, where the LAH4 peptide and plasmids were mixed and incubated for different times prior to transfection. The transfection efficiency was measured using flow cytometry to identify cells expressing GFP protein.

FIG. 10G discloses titers of AAV9 capsids produced in HEK293 cells in Ambr15 minibioreactors (15 mL volume) when transfected with plasmids for producing rAAV and providing an AAV genome vector containing an GFP expression cassette into HEK293 cells using the LAH4 peptide, where the LAH4 peptide and plasmids were mixed and incubated for different times prior to transfection. The AAV titers were quantified using ddPCR.

FIG. 10H shows the viability of HEK293 cells in Ambr15 minibioreactors (15 mL volume) and transfected with plasmids for producing AAV9 capsids and providing an AAV genome vector containing an GFP expression cassette using the LAH4 peptide, where the LAH4 peptide and plasmids were mixed and incubated for different times prior to transfection. The cell viabilities were quantified using an automated cell counter and Trypan Blue exclusion.

FIG. 11 shows titers of AAV9 capsids produced in HEK293 cells in 3 L bioreactors and transfected with plasmids for producing AAV9 capsids and providing an AAV genome vector containing a gene of interest using the LAH4 peptide. The AAV titers were quantified using ddPCR.

FIG. 12 shows the transfection efficiency of different HRPs (His-PTD4-LAH4, PTD4-LAH4, LAH4(W), and AAV2 VP1-2 BR3-spacer-LAH4) at different concentrations. HEK293 cells in wells (0.5 mL) of a 96 deep well plate were transfected with plasmids for producing rAAV and providing an AAV genome vector containing an GFP expression cassette using the different HRPs at different concentrations. At 24 hours post transfection, the transfection efficiency was measured using flow cytometry to identify cells expressing GFP protein.

FIG. 13 shows the transfection efficiency of different HRPs (His-PTD4-LAH4-L1-F4, PTD4-LAH4-L1-F4, LAH4-L1-F4(W), AAV2 VP1-2 BR3-spacer-LAH4-L1-F4 and SV40-T-NLS-spacer-LAH4-L1-F4) at different concentrations. HEK293 cells in wells (0.5 mL) of a 96 deep well plate were transfected with plasmids for producing rAAV and providing an AAV genome vector containing an GFP expression cassette using the different HRPs at different concentrations. At 24 hours post transfection, the transfection efficiency was measured using flow cytometry to identify cells expressing GFP protein.

FIG. 14 shows the AAV9 capsid titers of different HRPs (His-PTD4-LAH4, PTD4-LAH4, LAH4(W), and AAV2 VP1-2 BR3-spacer-LAH4) at different concentrations. HEK293 cells in wells (0.5 mL) of a 96 deep well plate were transfected with plasmids for producing rAAV and providing an AAV genome vector containing an GFP expression cassette using the different HRPs at different concentrations. After a predetermined time, AAV9 capsids were isolated from the cultures and titers were quantified using ddPCR.

FIG. 15 shows the AAV9 capsid titers of different HRPs (His-PTD4-LAH4-L1-F4, PTD4-LAH4-L1-F4, LAH4-L1-F4(W), and SV40-T-NLS-spacer-LAH4-L1-F4) at different concentrations. HEK293 cells in wells (0.5 mL) of a 96 deep well plate were transfected with plasmids for producing rAAV and providing an AAV genome vector containing an GFP expression cassette using the different HRPs at different concentrations. After a predetermined time, AAV9 capsids were isolated from the cultures and titers were quantified using ddPCR.

FIG. 16 shows the AAV9 capsid titers of HEK293 cells at large scale (e.g., 100 L) using the LAH4 peptide. HEK293 cells in 100 L bioreactor were transfected with plasmids for producing rAAV and providing an AAV genome vector. After a predetermined time, AAV9 capsids were isolated from the cultures and AAV titers were quantified using ddPCR.

FIG. 17 shows the percentage of Sf9 cells expressing GFP when initially transfected with a plasmid having a GFP expression cassette using different HRPs and a control transfection reagent (CELLFECTIN®, Thermo Fisher) at 24 hours post transfection. The transfection efficiency was measured using flow cytometry identify cells expressing GFP protein.

FIG. 18 shows the titers of rBVs produced in Sf9 cells by transfecting bacmids for producing rAAV using different HRPs and the control transfection reagent.

FIG. 19 shows the titers of rAAV produced in Sf9 cells infected with the rBVs generated using HRPs and the control transfection reagent from FIG. 13 and rBVs providing an AAV genome vector with a gene of interest. The AAV titers were quantified using ddPCR.

### DETAILED DESCRIPTION OF THE INVENTION

As required, detailed embodiments of the present disclosure are disclosed herein; however, it is to be understood that the disclosed embodiments are merely exemplary and may be embodied in various and alternative forms.

Except in the examples, or where otherwise expressly indicated, all numerical quantities in this description indicating amounts of material or conditions of reaction and/or use are to be understood as modified by the word “about”. For example, description referring to “about X” includes description of “X.” In one example, the term “about” is understood as within a range of normal tolerance in the art, for example within 2 standard deviations of the mean. In different examples, “about” refers a variability of ±0.0001%, ±0.0005%, ±0.001%, ±0.005%, ±0.01%, ±0.05%, ±0.1%, ±0.5%, ±1%, ±5%, or ±10%. In further examples, “about” can be understood as within ±9%, ±8%, ±7%, ±6%, ±5%, ±4%, ±3%, or ±2%.

Unless otherwise clear from context, all numerical values provided herein are modified by the term about. All ranges include the endpoints of the ranges. The first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

Unless indicated otherwise, all technical and scientific terms used herein have the same meaning as commonly understood by one of ordinary skill in the art to which the present disclosure belongs.

It is also to be understood that this disclosure is not limited to the specific embodiments and methods described below, as specific components and/or conditions may, of course, vary. Furthermore, the terminology used herein is used only for describing particular embodiments and is not intended to be limiting in any way.

Throughout this application, where publications are referenced, the disclosures of these publications in their entireties are hereby incorporated by reference into this application to more fully describe the state of the art to which this invention pertains.

It is preferably also be noted that, as used in the specification and the appended claims, the singular form “a”, “an”, and “the” comprise plural referents unless the context clearly indicates otherwise. For example, reference to a component in the singular is intended to comprise a plurality of components.

The terms “or” and “and” can be used interchangeably and can be understood to mean “and/or”.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

The terms “comprising”, “consisting of”, and “consisting essentially of” can be alternatively used. When one of these three terms is used, the presently disclosed and claimed subject matter can include the use of either of the other two terms.

In accordance with the present invention, the inventors have surprisingly discovered that cationic peptides identified as histidine rich peptides (HRPs) as well as other cationic peptides are capable of effectively transfecting cells to produce greater titers of recombinant adeno associated virus (rAAV) in different cell types such as human embryonic kidney 293 (HEK293) cells. To this end, the use of HRPs as a transfection reagent unexpectedly solves limitations for scaling the productions of biologic materials (e.g., recombinant proteins, nucleotides such as antisense oligonucleotides, small interfering ribonucleic acid (siRNA), micro RNA (miRNA), biologics, and gene therapy vectors such as rAAV and recombinant lentivirus), which have not been addressed by the current state of the art and has been a long felt need in the industry. Furthermore for some production types including insect cell productions, HRPs are prepared according to good manufacturing practices (GMP), whereas other transfection reagents are not capable of or are not being generated as GMP grade. HRPs are also biodegradable and has limited to no impairment/effect on cell viabilities.

Although the methods described herein may be disclosed and described as step(s), it is to be understood that the methods are not necessarily limited by the order of steps, as some steps may, in accordance with these methods, occur in different orders, and/or concurrently with other step(s) described herein and/or known in the art.

The rAAV production methods of various embodiments include the steps of co-transfecting cells with one or more vectors for rAAV production using a cationic peptide/HRP, culturing the transfected cells to generate rAAV, and recovering the rAAV. The term “cationic peptide/HRP” refers to HRPs as well as other described cationic peptides. For example, the cationic peptides include an amino acid sequence that is at least 85%, 90%, 95%, 99%, 99%+, or 100% identical to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124. The terms “percent identity”, “percent identical”, or “% identical to” in the context of two or more nucleotide or peptide amino acid sequences, refer to the percentage of nucleotides or amino acid residues that are the same, when compared and aligned for maximum correspondence. For example, percent identity is determined using NCBI blastp (amino acids) using the default settings.

In other examples, cationic peptides having an amino acid sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acid substitutions such as conserved amino acid exchanges. Illustrative examples for conserved amino acid exchanges are amino acid substitutions that maintain structural and/or functional properties of the amino acids' side-chains, e.g., an aromatic amino acid is substituted for another aromatic amino acid, an acidic amino acid is substituted for another acidic amino acid, a basic amino acid is substituted for another basic amino acid, and an aliphatic amino acid is substituted for another aliphatic amino acid. In some embodiments, a conservative amino acid substitution is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Standardized and accepted functionally equivalent amino acid substitutions are presented in Table 1. In contrast, examples of non-conserved amino acid exchanges are amino acid substitutions that do not maintain structural and/or functional properties of the amino acids' side-chains, e.g., an aromatic amino acid is substituted for a basic, acidic, or aliphatic amino acid, an acidic amino acid is substituted for an aromatic, basic, or aliphatic amino acid, a basic amino acid is substituted for an acidic, aromatic or aliphatic amino acid, and an aliphatic amino acid is substituted for an aromatic, acidic or basic amino acid.

TABLE 1
Conservative Amino Acid Substitutions

| Amino Acid Group | Conservative Substitutions |
|---|---|
| Nonpolar side chains | alanine, valine, leucine, glycine, |
| | isoleucine, proline, phenylalanine, |
| | methionine, tryptophan |
| Uncharged polar side chains | asparagine, glutamine, serine, threonine, |
| | tyrosine, cysteine |
| Beta-branched side chains | threonine, valine, isoleucine |
| Aromatic side chains | tyrosine, phenylalanine, tryptophan, |
| | histidine |
| Basic side chains or | lysine, arginine, histidine |
| positively charged R groups | |
| Acidic side chains or | aspartic acid, glutamic acid |
| negatively charged R groups | |

In other embodiments, the rAAV production methods include the steps of transiently co-transfecting cells suspended in a culture volume of more than 500 liters (L) with one or more vectors for rAAV production using a transfection reagent, culturing the transfected cells to generate rAAV, and optionally, recovering the rAAV. As previously noted, the invention solves limitations in current state of the art with cultures that limit the ability for rAAV or other biologic productions at scales of more than 500 L. For example, the current state of the art for rAAV or other biologic productions using HEK293 cells is limited to 500 L or less. Accordingly, the invention as described in this application overcomes the limitations of the current state of the art.

The rBV production methods of various embodiments include the steps of transfecting cells with recombinant bacmids having a baculovirus genome and a heterologous nucleotide sequence using a cationic peptide/HRP, culturing the transfected cells to generate rBV and optionally, recovering the rBV.

In other embodiments, the rBV production methods include the steps of transfecting cells with a heterologous nucleotide sequence using a cationic peptide such as an HRP, culturing the transfected cells to generate rBV, and optionally, recovering the rBV. The cells have at least a portion of a baculovirus genome. During the culturing step, the heterologous nucleotide sequence and the at least a portion of a baculovirus genome combine to form a baculovirus genome capable of generating rBV.

In other embodiments, the rBV production methods include the steps of co-transfecting cells with a complete or partial baculovirus genome, either circular or linearized, and with a heterologous nucleotide sequence (which can be incorporated in a transfer vector) containing a region or regions homologous to the baculovirus genome, using a cationic peptide such as an HRP, culturing the transfected cells to generate rBV, and optionally, recovering the rBV. During the culturing step, the complete or partial baculovirus genome and the heterologous nucleotide sequence recombine via the homologous regions to form a recombinant baculovirus genome capable of generating rBV, carrying at least a portion of the heterologous nucleotide sequence.

The rBV production methods of various embodiments include the steps of transfecting cells with recombinant bacmids having a nucleotide sequence providing an AAV genome vector or encoding Rep or Cap proteins using a cationic peptide/HRP, culturing the transfected cells to generate rBV, and optionally, recovering the rBV.

In other embodiments, the rBV production methods include the steps of transfecting cells with a nucleotide sequence providing an AAV genome vector or encoding Rep or Cap proteins using a cationic peptide such as an HRP, culturing the transfected cells to generate rBV, and optionally, recovering the rBV. The cells have at least a portion of a baculovirus genome. During the culturing step, the nucleotide sequence and the at least a portion of a baculovirus genome combine to form a baculovirus genome capable of generating rBV.

In other embodiments, the rBV production methods include the steps of co-transfecting cells with a complete or partial baculovirus genome, either circular or linearized, and with a heterologous nucleotide sequence (which can be incorporated in a transfer vector) containing either a rAAV vector genome or AAV rep and/or cap genes, and also containing a region or regions homologous to the baculovirus genome, using a cationic peptide such as an HRP, culturing the transfected cells to generate rBV, and optionally, recovering the rBV. During the culturing step, the complete or partial baculovirus genome and the heterologous nucleotide sequence recombine via the homologous regions to form a recombinant baculovirus genome capable of generating rBV, carrying at least a portion of the heterologous nucleotide sequence.

The cell generating methods of various embodiments include the steps of transfecting cells with a vector with a nucleotide sequence encoding an element used for generating rAAV using a cationic peptide/HRP and isolating a transfected cell having a genome with the nucleotide sequence.

In various embodiments of the rAAV production methods, the one or more vectors for rAAV production of any embodiment are co-transfected into at least 1 cell. In various embodiments, the one or more vectors for rAAV production of any embodiment are co-transfected into at least 1%, at least 5%, at least 5%, at least 10%, at least 15%, at least 20%, at least 25%, at least 30%, at least 35%, at least 40%, at least 45%, at least 50%, at least 55%, at least 60%, at least 65%, at least at least 70%, at least 75%, at least 80%, at least 85%, at least 90%, at least 95%, at least 99%, at least 99%+, or at least 100% of the cells. In further embodiments, the percentage of cells transfected with the one or more vectors is a range between any two percentages provided above.

In various embodiments of the rAAV production methods, the percentage of cells transfected with the one or more vectors for rAAV production using a cationic peptide/HRP is greater than a percentage of cells transfected with the one or more vectors for rAAV production using transfection reagents(s) including polyethylenimine or derivatives thereof, where transfections conditions are optimized for maximizing the percentage of cells transfected with one or more vectors or alternatively, under the same of similar transfections conditions.

In various embodiments of the rAAV production methods, the one or more vectors for rAAV production of any embodiment and one of the cationic peptide/HRP and transfection reagent of any embodiment are added to cells in a volume that is less than 10%, less than 9%, less than 8%, less than 7%, less than 6%, less than 5%, less than 4%, less than 3%, less than 2%, or less than 1% of a volume in which the cells are in culture. In various embodiments, the one or more vectors for rAAV production of any embodiment and one of the cationic peptide/HRP and transfection reagent of any embodiment are added to cells in a volume that is 0%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of a volume in which the cells are in culture. In further embodiments, the volume of the mixture of one or vectors and at least one of the cationic peptide/HRP and transfection reagent is a fraction of the volume in which the cells are in culture where the fraction is a range between any two percentages provided above.

In various embodiments of the rAAV production methods, the one or more vectors for rAAV production of any embodiment and one of the cationic peptide/HRP and transfection reagent of any embodiment are mixed together prior to the co-transfecting step. The mixture is stable for extended periods of time allowing for transfections of cells in larger cell culture volumes. In various embodiments, the mixture of the one or more vectors and peptide/HRP and transfection reagent of any embodiment are incubated for at least 1 minute, at least 5 minutes, at least 10 minutes, at least 20 minutes, at least 50 minutes, at least 100 minutes, at least 200 minutes, at least 500 minutes, at least 1000 minutes, or at least 2000 minutes prior to the co-transfecting step. In various embodiments, the mixture of the one or more vectors and peptide/HRP and transfection reagent of any embodiment are incubated for 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 120 minutes, 130 minutes, 140 minutes, 150 minutes, 160 minutes, 170 minutes, 180 minutes, 190 minutes, 200 minutes, 210 minutes, 220 minutes, 230 minutes, 240 minutes, 250 minutes, 260 minutes, 270 minutes, 280 minutes, 290 minutes, 300 minutes, 310 minutes, 320 minutes, 330 minutes, 340 minutes, 350 minutes, 360 minutes, 370 minutes, 380 minutes, 390 minutes, 400 minutes, 410 minutes, 420 minutes, 430 minutes, 440 minutes, 450 minutes, 460 minutes, 470 minutes, 480 minutes, 490 minutes, 500 minutes, 510 minutes, 520 minutes, 530 minutes, 540 minutes, 550 minutes, 560 minutes, 570 minutes, 580 minutes, 590 minutes, 600 minutes, 700 minutes, 800 minutes, 900 minutes, 1000 minutes, 1500 minutes, or 2000 minutes prior to the co-transfecting step. In various embodiments, the incubation time is a range between any two times provided above.

In various embodiments of the rAAV production methods, the percentage of cells transfected with the one or more vectors for rAAV production using a cationic peptide/HRP is greater than a percentage of cells transfected with the one or more vectors for rAAV production using transfection reagents(s) including polyethylenimine or derivatives thereof, wherein the cationic peptide/HRP and the transfection reagents(s) including polyethylenimine or derivatives thereof are mixed and incubated with the one or more vectors for the same time period prior to the co-transfecting step. In various embodiment, the percentage of cells transfected with the one or more vectors for rAAV production using a cationic peptide/HRP is at least 1%, at least 10%, at least 50%, at least 100%, at least 500%, at least 1000%, at least 5000%, or at least 10000% greater than a percentage of cells transfected with the one or more vectors for rAAV production using transfection reagents(s) including polyethylenimine or derivatives thereof, wherein the cationic peptide/HRP and the transfection reagents(s) including polyethylenimine or derivatives thereof are mixed and incubated with the one or more vectors for the same time period prior to the co-transfecting step.

In various embodiments of the rAAV production methods, the cells transfected with the one or more vectors for rAAV production using a cationic peptide/HRP have a specific productivity (vector genome (vg)/cell) that is greater than a specific productivity of cells transfected with the one or more vectors for rAAV production using transfection reagents(s) including polyethylenimine or derivatives thereof, where transfections conditions are optimized for maximizing the percentage of cells transfected with one or more vectors or alternatively, under the same of similar transfections conditions. Specific productivity can be assessed using digital droplet polymerase chain reaction (ddPCR) and calculated by dividing the final titer (vector genomes (vg)/mL of harvest fluid) by peak viable cell density (number of cells/mL). In various embodiments, the specific productivity of the cells transfected with the one or more vectors for rAAV production using a cationic peptide/HRP is at least at least 1%, at least 10%, at least 50%, at least 100%, at least 500%, at least 1000%, at least 5000%, at least 10000%, at least 3 log, at least 4 log, or at least 5 log greater than the specific productivity of cells transfected with the one or more vectors for rAAV production using transfection reagents(s) including polyethylenimine or derivatives thereof, where transfections conditions are optimized for maximizing the percentage of cells transfected with one or more vectors or alternatively, under the same of similar transfections conditions.

In various embodiments, the specific productivity of the cells transfected with the one or more vectors for rAAV production using a cationic peptide/HRP is at least 1×10² vector genome (vg)/cell, at least 1×10³ vg/cell, at least 1×10⁴ vg/cell, at least 1×10⁵ vg/cell, at least 1×10⁶ vg/cell, or at least 1×10⁷ vg/cell.

In various embodiments of the rAAV production methods, the cells transfected with the one or more vectors for rAAV production using a cationic peptide/HRP generate an rAAV titer that is greater than an rAAV titer of cells transfected with the one or more vectors for rAAV production using transfection reagents(s) including polyethylenimine or derivatives thereof, where transfections conditions including the cell culture volume are optimized for maximizing the percentage of cells transfected with one or more vectors or alternatively, under the same of similar transfections conditions.

In various embodiments of the rAAV production methods, the rAAV titer generated by cells transfected with the one or more vectors for rAAV production using a cationic peptide/HRP is at least 1%, at least 10%, at least 50%, at least 100%, at least 500%, at least 1000%, at least 5000%, at least 10000%, or at least 3 log greater than the rAAV titer generated by cells transfected with the one or more vectors for rAAV production using transfection reagents(s) including polyethylenimine or derivatives thereof, where transfections conditions including the cell culture volume are optimized for maximizing the percentage of cells transfected with one or more vectors or alternatively, under the same of similar transfections conditions.

In various embodiments of the rAAV production methods, the rAAV titer generated by cells transfected with the one or more vectors for rAAV production using a cationic peptide/HRP is at least 0.1×10¹⁰ vg/mL, at least 0.5×10¹⁰ vg/mL, at least 1×10¹⁰ vg/mL, at least 1×10¹¹ vg/mL, at least 1×10¹² vg/mL, at least 1×10¹³ vg/mL, or at least 1×10¹⁴ vg/mL.

In various embodiments of the rAAV production methods, the weight ratio of the one or more vectors for rAAV production of any embodiment to the one of the cationic peptide/HRP and transfection reagent of any embodiment is 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, or 1:50. In various embodiments, the weight ratio of the one or more vectors for rAAV production of any embodiment to the one of the cationic peptide/HRP and transfection reagent of any embodiment is a range between any two weight ratios provided above.

In various embodiments of the rAAV production methods, the concentration of cationic peptide/HRP in the cell culture is at least 0.001 microgram (μg)/mL, at least 0.01 μg/mL, at least 0.1 μg/mL, at least 0.5 μg/mL, at least 1 μg/mL, at least 10 μg/mL, at least 50 μg/mL, at least 100 μg/mL, 1-200 μg/mL, 5-50 μg/mL, 10-150 μg/mL, or 100-200 μg/mL. In various embodiments, the concentration of cationic peptide or HRP in the cell culture is 1 μg/mL, 1.5 μg/mL, 2 μg/mL, 2.5 μg/mL, 3 μg/mL, 3.5 μg/mL, 4 μg/mL, 4.5 μg/mL, 5 μg/mL, 5.5 μg/mL, 6, μg/mL, 6.5 μg/mL, 7 μg/mL, 7.5 μg/mL, 8 μg/mL, 8.5 μg/mL, 9 μg/mL, 9.5 μg/mL, 10 μg/mL, 10.5 μg/mL, 11 μg/mL, 11.5 μg/mL, 12 μg/mL, 12.5 μg/mL, 13 μg/mL, 13.5 μg/mL, 14 μg/mL, 14.5 μg/mL, 15 μg/mL, 15.5 μg/mL, 16 μg/mL, 16.5 μg/mL, 17 μg/mL, 17.5 μg/mL, 18 μg/mL, 18.5 μg/mL, 19 μg/mL, 19.5 μg/mL, 20 μg/mL, 20.5 μg/mL, 21 μg/mL, 21.5 μg/mL, 22 μg/mL, 22.5 μg/mL, 23 μg/mL, 23.5 μg/mL, 24 μg/mL, 24.5 μg/mL, 25 μg/mL, 26 μg/mL, 27 μg/mL, 28 μg/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 61 μg/mL, 62 μg/mL, 63 μg/mL, 64 μg/mL, 65 μg/mL, 66 μg/mL, 67 μg/mL, 68 μg/mL, 69 μg/mL, 70 μg/mL, 71 μg/mL, 72 μg/mL, 73 μg/mL, 74 μg/mL, 75 μg/mL, 76 μg/mL, 77 μg/mL, 78 μg/mL, 79 μg/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 μg/mL, 97 μg/mL, 98 μg/mL, 99 μg/mL, 100 μg/mL, 101 μg/mL, 102 μg/mL, 103 μg/mL, 104 μg/mL, 105 μg/mL, 106 μg/mL, 107 μg/mL, 108 μg/mL, 109 μg/mL, 110 μg/mL, 111 μg/mL, 112 μg/mL, 113 μg/mL, 114 μg/mL, 115 μg/mL, 116 μg/mL, 117 μg/mL, 118 μg/mL, 119 μg/mL, 120 μg/mL, 121 μg/mL, 122 μg/mL, 123 μg/mL, 124 μg/mL, 125 μg/mL, 126 μg/mL, 127 μg/mL, 128 μg/mL, 129 μg/mL, 130 μg/mL, 131 μg/mL, 132 μg/mL, 133 μg/mL, 134 μg/mL, 135 μg/mL, 136 μg/mL, 137 μg/mL, 138 μg/mL, 139 μg/mL, 140 μg/mL, 141 μg/mL, 142 μg/mL, 143 μg/mL, 144 μg/mL, 145 μg/mL, 146 μg/mL, 147 μg/mL, 148 μg/mL, 149 μg/mL, 150 μg/mL, 151 μg/mL, 152 μg/mL, 153 μg/mL, 154 μg/mL, 155 μg/mL, 156 μg/mL, 157 μg/mL, 158 μg/mL, 159 μg/mL, 160 μg/mL, 161 μg/mL, 162 μg/mL, 163 μg/mL, 164 μg/mL, 165 μg/mL, 166 μg/mL, 167 μg/mL, 168 μg/mL, 169 μg/mL, 170 μg/mL, 171 μg/mL, 172 μg/mL, 173 μg/mL, 174 μg/mL, 175 μg/mL, 176 μg/mL, 177 μg/mL, 178 μg/mL, 179 μg/mL, 180 μg/mL, 181 μg/mL, 182 μg/mL, 183 μg/mL, 184 μg/mL, 185 μg/mL, 186 μg/mL, 187 μg/mL, 188 μg/mL, 189 μg/mL, 190 μg/mL, 191 μg/mL, 192 μg/mL, 193 μg/mL, 194 μg/mL, 195 μg/mL, 196 μg/mL, 197 μg/mL, 198 μg/mL, 199 μg/mL, or 200 μg/mL. In various embodiments, the concentration of the cationic peptide/HRP in the cell culture is a range between any two concentrations provided above.

In various embodiments of the rAAV production methods, the concentration of the one or more vectors for rAAV production in the cell culture is at least 0.0001 μg/ml, at least 0.001 μg/ml, at least 0.01 μg/ml, at least 0.1 μg/ml, at least 0.5 μg/ml, at least 1, μg/ml, 0.1-15 μg/ml, or 0.5-200 μg/ml. In various embodiments, the concentration of the one or more vectors for rAAV production in the cell culture is 0.0001 μg/ml, 0.001 μg/ml, 0.01 μg/ml, 0.05 μg/ml, 0.1 μg/ml, 0.5 μg/ml, 1 μg/ml, 1.5 μg/ml, 2 μg/ml, 2.5 μg/ml, 3 μg/ml, 3.5 μg/ml, 4 μg/ml, 4.5 μg/ml, 5 μg/ml, 5.5 μg/ml, 6, μg/ml, 6.5 μg/ml, 7 μg/ml, 7.5 μg/ml, 8 μg/ml, 8.5 μg/ml, 9 μg/ml, 9.5 μg/ml, 10 μg/ml, 10.5 μg/ml, 11 μg/ml, 11.5 μg/ml, 12 μg/ml, 12.5 μg/ml, 13 μg/ml, 13.5 μg/ml, 14 μg/ml, 14.5 μg/ml, 15 μg/ml, 15.5 μg/ml, 16 μg/ml, 16.5 μg/ml, 17 μg/ml, 17.5 μg/ml, 18 μg/ml, 18.5 μg/ml, 19 μg/ml, 19.5 μg/ml, 20 μg/ml, 20.5 μg/ml, 21 μg/ml, 21.5 μg/ml, 22 μg/ml, 22.5 μg/ml, 23 μg/ml, 23.5 μg/ml, 24 μg/ml, 24.5 μg/ml, 25 μg/ml, 26 μg/mL, 27 μg/mL, 28 μg/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 61 μg/mL, 62 μg/mL, 63 μg/mL, 64 μg/mL, 65 μg/mL, 66 μg/mL, 67 μg/mL, 68 μg/mL, 69 μg/mL, 70 μg/mL, 71 μg/mL, 72 μg/mL, 73 μg/mL, 74 μg/mL, 75 μg/mL, 76 μg/mL, 77 μg/mL, 78 μg/mL, 79 μg/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 μg/mL, 97 μg/mL, 98 μg/mL, 99 μg/mL, 100 μg/mL, 101 μg/mL, 102 μg/mL, 103 μg/mL, 104 μg/mL, 105 μg/mL, 106 μg/mL, 107 μg/mL, 108 μg/mL, 109 μg/mL, 110 μg/mL, 111 μg/mL, 112 μg/mL, 113 μg/mL, 114 μg/mL, 115 μg/mL, 116 μg/mL, 117 μg/mL, 118 μg/mL, 119 μg/mL, 120 μg/mL, 121 μg/mL, 122 μg/mL, 123 μg/mL, 124 μg/mL, 125 μg/mL, 126 μg/mL, 127 μg/mL, 128 μg/mL, 129 μg/mL, 130 μg/mL, 131 μg/mL, 132 μg/mL, 133 μg/mL, 134 μg/mL, 135 μg/mL, 136 μg/mL, 137 μg/mL, 138 μg/mL, 139 μg/mL, 140 μg/mL, 141 μg/mL, 142 μg/mL, 143 μg/mL, 144 μg/mL, 145 μg/mL, 146 μg/mL, 147 μg/mL, 148 μg/mL, 149 μg/mL, 150 μg/mL, 151 μg/mL, 152 μg/mL, 153 μg/mL, 154 μg/mL, 155 μg/mL, 156 μg/mL, 157 μg/mL, 158 μg/mL, 159 μg/mL, 160 μg/mL, 161 μg/mL, 162 μg/mL, 163 μg/mL, 164 μg/mL, 165 μg/mL, 166 μg/mL, 167 μg/mL, 168 μg/mL, 169 μg/mL, 170 μg/mL, 171 μg/mL, 172 μg/mL, 173 μg/mL, 174 μg/mL, 175 μg/mL, 176 μg/mL, 177 μg/mL, 178 μg/mL, 179 μg/mL, 180 μg/mL, 181 μg/mL, 182 μg/mL, 183 μg/mL, 184 μg/mL, 185 μg/mL, 186 μg/mL, 187 μg/mL, 188 μg/mL, 189 μg/mL, 190 μg/mL, 191 μg/mL, 192 μg/mL, 193 μg/mL, 194 μg/mL, 195 μg/mL, 196 μg/mL, 197 μg/mL, 198 μg/mL, 199 μg/mL, or 200 μg/mL. In various embodiments, the concentration of the one or more vectors for rAAV production in the cell culture is a range between any two concentrations provided above.

In various embodiments of the rAAV production methods, the one or more vectors for rAAV production of any embodiment include an AAV genome vector, an AAV helper vector, and a vector having a non-AAV helper function-providing nucleotide. Examples the one or more vectors for rAAV production includes nucleotide sequences in the form of plasmids or cosmids that when transfected into a cell express Rep and Cap proteins, provide for the generation of AAV genome vectors, and provide viral helper functions (e.g., transcription factors, etc.) for generating rAAV.

In various embodiments of the rAAV production methods and cell generating methods, the cells are eukaryotic cells such as mammalian cells. Examples of mammalian cells include human cells. Other examples of mammalian cells include HEK293, HeLa, CHO, NS0, SP2/0, PER.C6, Vero, RD, BHK, HT 1080, A549, Cos-7, ARPE-19, or MRC-5 cells.

In various embodiments of the rBV production methods, the recombinant bacmids of any embodiment and cationic peptide/HRP of any embodiment are mixed together prior to the transfecting step.

In various embodiments of the rBV production methods, the recovery step includes recovering baculovirus infected insect cells (BIICs). In various embodiments of the rBV production methods, the recovery step comprises recovering BIICs and the recovered BIICs are cultured with cells previously uninfected with baculovirus in culture to generate rAAV.

In various embodiments of the rBV production methods and cell generating methods, the cells are insect cells derived from Spodoptera frugiperda, Aedes albopictus, Bombyxmori, Trichoplusia ni, Ascalapha odorata, Drosphila, Anophele, Culex, or Aedes. Examples of insect cells include Sf9, High Five, Se301, SeIZD2109, SeUCR1, Sf900+, Sf21, BTI-TN-5B1-4, MG-1, Tn368, HzAm1, BM-N, Ha2302, Hz2E5 or Ao38 cells.

In various embodiments of the rAAV or rBV production methods, the cell culture is produced in shake flasks or bioreactors with volumes of at least 1 mL, at least 10 mL, at least 20 mL, at least 50 mL, at least 100 mL, at least 500 mL, at least 1 liter (L), at least 10 L, at least 50 L, at least 100 L, at least 250 L, at least 500 L, at least 1000 L, at least 1500 L, at least 2000 L, or at least 2500 L. In various embodiments, the cell culture volume is 1 mL, 10 mL, 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, 5 L, 6 L, 7 L, 8 L, 9 L, 10 L, 11 L, 12 L, 13L, 14 L, 15 L, 16 L, 17 L, 18 L, 19 L, 20 L, 21 L, 22 L, 23 L, 24 L, 25 L, 26 L, 27 L, 28 L, 29 L, 30 L, 31 L, 32 L, 33 L, 34 L, 35 L, 36 L, 37 L, 38 L, 39 L, 40 L, 41 L, 42 L, 43 L, 44 L, 45 L, 46 L, 47 L, 48 L, 49 L, 50 L, 51 L, 52 L, 53 L, 54 L, 55 L, 56 L, 57 L, 58 L, 59 L, 60 L, 61 L, 62 L, 63 L, 64 L, 65 L, 66 L, 67 L, 68 L, 69 L, 70 L, 71 L, 72 L, 73 L, 74 L, 75 L, 76 L, 77 L, 78 L, 79 L, 80 L, 81 L, 82 L, 83 L, 84 L, 85 L, 86 L, 87 L, 88 L, 89 L, 90 L, 91 L, 92 L, 93 L, 94 L, 95 L, 96 L, 97 L, 98 L, 99 L, 100 L, 110 L, 120 L, 130 L, 140 L, 150 L, 160 L, 170 L, 180 L, 190 L, 200 L, 210 L, 220 L, 230 L, 240 L, 250 L, 260 L, 270 L, 280 L, 290 L, 300 L, 310 L, 320 L, 330 L, 340 L, 350 L, 360 L, 370 L, 380 L, 390 L, 400 L, 410 L, 420 L, 430 L, 440 L, 450 L, 460 L, 470 L, 480 L, 490 L, 500 L, 510 L, 520 L, 530 L, 540 L, 550 L, 560 L, 570 L, 580 L, 590 L, 600 L, 610 L, 620 L, 630 L, 640 L, 650 L, 660 L, 670 L, 680 L, 690 L, 700 L, 710 L, 720 L, 730 L, 740 L, 750 L, 760 L, 770 L, 780 L, 790 L, 800 L, 810 L, 820 L, 830 L, 840 L, 850 L, 860 L, 870 L, 880 L, 890 L, 900 L, 910 L, 920 L, 930 L, 940 L, 950 L, 960 L, 970 L, 980 L, 990 L, 1000 L, 1010 L, 1020 L, 1030 L, 1040 L, 1050 L, 1060 L, 1070 L, 1080 L, 1090 L, 1100 L, 1110 L, 1120 L, 1130 L, 1140 L, 1150 L, 1160 L, 1170 L, 1180 L, 1190 L, 1200 L, 1210 L, 1220 L, 1230 L, 1240 L, 1250 L, 1260 L, 1270 L, 1280 L, 1290 L, 1300 L, 1310 L, 1320 L, 1330 L, 1340 L, 1350 L, 1360 L, 1370 L, 1380 L, 1390 L, 1400 L, 1410 L, 1420 L, 1430 L, 1440 L, 1450 L, 1460 L, 1470 L, 1480 L, 1490 L, 1500 L, 1510 L, 1520 L, 1530 L, 1540 L, 1550 L, 1560 L, 1570 L, 1580 L, 1590 L, 1600 L, 1610 L, 1620 L, 1630 L, 1640 L, 1650 L, 1660 L, 1670 L, 1680 L, 1690 L, 1700 L, 1710 L, 1720 L, 1730 L, 1740 L, 1750 L, 1760 L, 1770 L, 1780 L, 1790 L, 1800 L, 1810 L, 1820 L, 1830 L, 1840 L, 1850 L, 1860 L, 1870 L, 1880 L, 1890 L, 1900 L, 1910 L, 1920 L, 1930 L, 1940 L, 1950 L, 1960 L, 1970 L, 1980 L, 1990 L, 2000 L, 2010 L, 2020 L, 2030 L, 2040 L, 2050 L, 2060 L, 2070 L, 2080 L, 2090 L, 2100 L, 2110 L, 2120 L, 2130 L, 2140 L, 2150 L, 2160 L, 2170 L, 2180 L, 2190 L, 2200 L, 2210 L, 2220 L, 2230 L, 2240 L, 2250 L, 2260 L, 2270 L, 2280 L, 2290 L, 2300 L, 2310 L, 2320 L, 2330 L, 2340 L, 2350 L, 2360 L, 2370 L, 2380 L, 2390 L, 2400 L, 2410 L, 2420 L, 2430 L, 2440 L, 2450 L, 2460 L, 2470 L, 2480 L, 2490 L, 2500 L, 2510 L, 2520 L, 2530 L, 2540 L, 2550 L, 2560 L, 2570 L, 2580 L, 2590 L, 2600 L, 2610 L, 2620 L, 2630 L, 2640 L, 2650 L, 2660 L, 2670 L, 2680 L, 2690 L, 2700 L, 2710 L, 2720 L, 2730 L, 2740 L, 2750 L, 2760 L, 2770 L, 2780 L, 2790 L, 2800 L, 2810 L, 2820 L, 2830 L, 2840 L, 2850 L, 2860 L, 2870 L, 2880 L, 2890 L, 2900 L, 2910 L, 2920 L, 2930 L, 2940 L, 2950 L, 2960 L, 2970 L, 2980 L, 2990 L, or 3000 L. In further embodiments, the culture volume is a range between any two volumes provided above.

In various embodiments, the complete or partial baculovirus genome is circular or linearized baculovirus genome that is, upon recombination with a heterologous sequence, capable of generating rBV. In one embodiment, the method utilizes the circular baculovirus genome as the frequency of recombination with the heterologous sequence carrying the homologous region or regions is low. In another embodiment, the method utilizes the linearized baculovirus genome, which decreases the generation of non-recombinant baculoviruses and increases the frequency of recombination with the said heterologous sequence, thereby improving the probability of generating recombinant baculoviruses. In a further embodiment, the method utilizes the linearized baculovirus genome lacking one or more essential baculovirus genes or their parts, thereby preventing generation of non-recombinant baculoviruses. The complementation of the essential genes occurs upon recombination with the said heterologous sequence and circularization of the baculovirus genome, assuring generation of recombinant baculoviruses with a high frequency.

In various embodiments, the heterologous nucleotide sequence or the nucleotide sequence providing an rAAV genome vector or encoding Rep or Cap proteins and carrying a region or regions homologous to the baculovirus genome, is present in linear or circular form or integrated within a transfer vector.

In various embodiments, co-transfection into the cells of the complete or partial baculovirus genome and at least one heterologous nucleotide or the nucleotide sequence providing an rAAV genome vector or encoding Rep or Cap proteins, and carrying the region or regions homologous to the baculovirus genome, results in homologous recombination of the at least one heterologous nucleotide or the nucleotide sequence providing an AAV genome vector or encoding Rep or Cap proteins into the complete or partial baculovirus genome.

In various embodiments, co-transfection into the cells of the complete or partial baculovirus genome and at least one heterologous nucleotide or the nucleotide sequence providing an rAAV genome vector or encoding Rep or Cap proteins, and carrying the region or regions homologous to the baculovirus genome, results in homologous recombination of the ends of the complete or partial, linearized baculovirus genome and at least one said heterologous nucleotide or the nucleotide sequence providing an AAV genome vector or encoding Rep or Cap proteins, such that a circularized recombinant baculovirus genome is formed.

In various embodiments of the cell generating methods, the vector includes an expression control element that is operably linked to the nucleotide such that the expression control element controls expression of the element for producing rAAV.

In various embodiments of the cell generating methods, the vector includes a nucleotide encoding a selection element. The methods include the step of applying a selection pressure (e.g., antibiotic) specific for the selection element (e.g., antibiotic resistance protein) prior to the isolation step. Alternatively, the methods include the step of identifying (e.g., emitting a fluorescence wavelength to the cells via flow cytometry) the selection element (e.g., fluorescent protein) prior to the isolation step.

In various embodiments of the cell generating methods, the element for producing rAAV is an element providing an AAV helper function or an element providing an AAV non-helper function.

In various embodiments, at least a portion of the cationic peptide/HRP of any embodiment has a α-helical conformation comprising hydrophobic amino acid residues positioned on a side of the conformation and polar amino acid residues positioned on an opposing side of the conformation during transfection. For example, linear cationic peptide/HRP can form into a-helical conformation when in the presence of cell membranes.

In various embodiments, the polar amino acid residues include a histidine residue(s) (His or H). Other examples of a polar amino acid residue include glutamine (Gln or Q), asparagine (Asn or N), serine (Ser or S), threonine (Thr or T), tyrosine (Tyr or Y), and cysteine (Cys or C).

In various embodiments, the hydrophobic amino acid residues include an alanine residue(s) (Ala or A) or leucine residue(s) (Leu or L). Other examples of a hydrophobic amino acid residue include methionine (Met or M), phenylalanine (Phe or F), valine (Val or V), Proline (Pro or P), or glycine (Gly or G).

In various embodiments, the cationic peptide/HRP of any embodiment has N-terminal or C-terminal end portions having an amino acid residue that is positively charged at a neutral pH (e.g., pH 7.4) such as a lysine or arginine residue. For example, the positively charged amino acid can be positioned at the N-terminus or C-terminus of the cationic peptide/HRP.

In various embodiments, the cationic peptide/HRP of any embodiment comprises tyrosine (Tyr or Y) or tryptophan (Trp or W).

In various embodiments, the cationic peptide/HRP of any embodiment includes at least 5 amino acid residues, at least 10 amino acid residues, at least 15 amino acid residues, at least 19 amino acid residues, at least 20 amino acid residues, at least 21 amino acid residues, at least 25 amino acid residues, at least 30 amino acid residues, at least 35 amino acid residues, at least 40 amino acid residues, at least 45 amino acid residues, or at least 50 amino acid residues. In various embodiments, the length of the cationic peptide/HRP is 10 amino acid residues, 11 amino acid residues, 12 amino acid residues, 13 amino acid residues, 14 amino acid residues, 15 amino acid residues, 16 amino acid residues, 17 amino acid residues, 18 amino acid residues, 19 amino acid residues, 20 amino acid residues, 21 amino acid residues, 22 amino acid residues, 23 amino acid residues, 24 amino acid residues, 25 amino acid residues, 26 amino acid residues, 27 amino acid residues, 28 amino acid residues, 29 amino acid residues, 30 amino acid residues, 31 amino acid residues, 32 amino acid residues, 33 amino acid residues, 34 amino acid residues, 35 amino acid residues, 36 amino acid residues, 37 amino acid residues, 38 amino acid residues, 39 amino acid residues, 40 amino acid residues, 41 amino acid residues, 42 amino acid residues, 43 amino acid residues, 44 amino acid residues, 45 amino acid residues, 46 amino acid residues, 47 amino acid residues, 48 amino acid residues, 49 amino acid residues, 50 amino acid residues, 51 amino acid residues, 52 amino acid residues, 53 amino acid residues, 54 amino acid residues, 55 amino acid residues, 56 amino acid residues, 57 amino acid residues, 58 amino acid residues, 59 amino acid residues, 60 amino acid residues, 61 amino acid residues, 62 amino acid residues, 63 amino acid residues, 64 amino acid residues, 65 amino acid residues, 66 amino acid residues, 67 amino acid residues, 68 amino acid residues, 69 amino acid residues, 70 amino acid residues, 71 amino acid residues, 72 amino acid residues, 73 amino acid residues, 74 amino acid residues, 75 amino acid residues, 76 amino acid residues, 77 amino acid residues, 78 amino acid residues, 79 amino acid residues, 80 amino acid residues, 81 amino acid residues, 82 amino acid residues, 83 amino acid residues, 84 amino acid residues, 85 amino acid residues, 86 amino acid residues, 87 amino acid residues, 88 amino acid residues, 89 amino acid residues, 90 amino acid residues, 91 amino acid residues, 92 amino acid residues, 93 amino acid residues, 94 amino acid residues, 95 amino acid residues, 96 amino acid residues, 97 amino acid residues, 98 amino acid residues, 99 amino acid residues, or 100 amino acid residues. In various embodiments, the length of the cationic peptide/HRP is a range between any two lengths provided above.

In various embodiments, the cationic peptide/HRP/transfection reagent of any embodiment is prepared according to good manufacturing practices.

In various embodiments, the cationic peptide/HRP and transfection reagent of any embodiment is biodegradable. The term “biodegradable” refers to a material that can be broken down or eroded by chemical (pH, hydrolysis, enzymatic action) and/or physical processes once added to cell culture and exposed to the physiological environment of the cell culture. The kinetics of this process can take from minutes to days.

### Adeno Associated Virus

In various embodiments, an rAAV capsid produced by a method of any embodiment is disclosed. The rAAV capsid has a concentration of VP1, VP2, or VP3 proteins that is greater than a concentration of VP1, VP2, or VP3 proteins of an rAAV capsid produced under the same conditions.

In various embodiments, an rAAV capsid produced by a method of any embodiment is disclosed.

The rAAV and rAAV capsids include rAAV particles disclosed in or may be made according to knowing methods, for example as taught in U.S. Pat. No. 9,504,762, WO 2018/022608, WO 2019/222136, and US 2019/0376081, the disclosures of which are hereby incorporated by reference in their entirety.

“AAV” is a standard abbreviation for adeno-associated virus. Adeno-associated virus is a single-stranded DNA parvovirus having a genome encapsulated by a capsid. There are currently thirteen serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to inverted terminal repeats (ITRs). The similar infectivity patterns also suggest that the replication functions in each serotype are under similar regulatory control.

An “AAV viral particle” as used herein refers to an infectious viral particle composed of at least one AAV capsid protein and an encapsidated AAV genome. “Recombinant AAV” or “rAAV”, “rAAV virion” or “rAAV viral particle” or “rAAV vector particle” or “AAV virus” refers to a viral particle composed of at least one capsid or Cap protein and an encapsidated rAAV vector genome (vg) as described herein. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of AAV vector particles necessarily includes production of rAAV vector, as such a vector is contained within an rAAV vector particle.

As used herein, the terms “heterologous gene”, “heterologous sequence”, “heterologous”, “heterologous regulatory sequence”, “heterologous transgene”, or “transgene” means that the referenced gene or regulatory sequence is not naturally present in the AAV vector or particle and has been artificially introduced therein. For example, these terms refer to a nucleic acid that comprises both a heterologous gene and a heterologous regulatory sequence that are operably linked to the heterologous gene that control expression of that gene in a host cell. It is contemplated that the transgene herein can encode a biomolecule (e.g., a therapeutic biomolecule), such as a protein (e.g., an enzyme), polypeptide, peptide, RNA (e.g., tRNA, dsRNA, ribosomal RNA, catalytic RNAs, siRNA, miRNA, pre-miRNA, lncRNA, snoRNA, small hairpin RNA, trans-splicing RNA, and antisense RNA), one or more components of a gene or base editing system, e.g., a CRISPR gene editing system, antisense oligonucleotides (AONs), antisense oligonucleotide (AON)-mediated exon skipping, a poison exon(s) that triggers nonsense mediated decay (NMD), or a dominant negative mutant.

The term “recombinant” refers nucleic acid molecules or proteins formed by using recombinant DNA techniques. For example, a recombinant nucleic acid molecule can be formed by combining nucleic acid sequences and sequence elements. A recombinant protein can be a protein that is produced by a cell that has received a recombinant nucleic acid molecule.

The terms “encodes,” “encoded” and “encoding” refer to the inherent property of specific sequences of nucleotides in a nucleic acid molecule, such as a gene, complementary DNA (cDNA), or messenger RNA (mRNA), to serve as templates for synthesis of other polymers and macromolecules in biological processes. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA.

“Capsid” refers to the structure in which the rAAV vector genome is packaged. The capsid includes VP1 proteins or VP3 proteins, but more typically, all three of VP1, VP2, and VP3 proteins, as found in native AAV. The sequence of the capsid proteins determines the serotype of the rAAV virions. rAAV virions include those derived from a number of AAV serotypes, including AAV1, AAV2, AAV4, AAV5, AAV6, AAV7, AAV8, AAV9, AAV10, AAV11, AAV12, AAV13, AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof (see, e.g., U.S. Pat. No. 8,318,480 for its disclosure of non-natural mixed serotypes). Exemplary capsids are also provided in International Application Publication No. WO 2018/022608 and WO 2019/222136, which are incorporated herein in its entirety. The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. In various embodiments, the capsid of the AAV viral particle has a VP1, VP2, or VP3 protein with an amino acid sequence that is at least about 50%, 55%, 60%, 65%, 70%, 75%, 80%, 85%, 90%, 95%, 96%, 97%, 98%, 99% or 100% identical to a portion of an amino acid sequence from AAV-1 (Genbank Accession No. AAD27757.1), AAV-2 (NCBI Reference Sequence No. YP_680426.1), AAV-3 (NCBI Reference Sequence No. NP_043941.1), AAV-3B (Genbank Accession No. AAB95452.1), AAV-4 (NCBI Reference Sequence No. NP_044927.1), AAV-5 (NCBI Reference Sequence No. YP_068409.1), AAV-6 (Genbank Accession No. AAB95450.1), AAV-7 (NCBI Reference Sequence No. YP_077178.1), AAV-8 (NCBI Reference Sequence No. YP_077179.1), AAV-9 (Genbank Accession No. AAS99264.1), AAV-10 (Genbank Accession No. AAT46337.1), AAV-11 (Genbank Accession No. AAT46339.1), AAV-12 (Genbank Accession No. ABI16639.1), AAV-13 (Genbank Accession No. ABZ10812.1), or any amino acid sequence disclosed in WO 2018/022608 and WO 2019/222136. Construction and use of AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In one embodiment, the rAAV particle is pseudotyped with an AAV capsid, wherein the VP1 protein comprises the amino acid sequence of any one of SEQ ID NOs:2-76; or comprises an amino acid sequence that is at least 60%, at least 65%, at least 70%, at least 75%, at least 80%, at least 85%, at least 90%, or at least 95% identical over the full length of any one of SEQ ID NOs: 2-76.

An AAV viral particle may be a “pseudotyped” or “hybrid” AAV viral particle. The terms “hybrid” and “pseudotyped” as they relate to AAV viral particles are used interchangeably herein and are intended to indicate that the Rep proteins, inverted terminal repeat sequences (ITRs) and/or capsid proteins are of different serotypes. A large number of alternative capsid variants have been identified from, for example, humans, baboons, pigs, marmosets, chimpanzees, and rhesus, pigtailed, and/or cynomolgus macaques, for example, as disclosed by U.S. Pat. No. 9,737,618; and Gao, G. et al., Clades of Adeno-associated viruses are widely disseminated in human tissues, J. Virol., 78(12):6381-8 (2004), each of which is incorporated herein by reference in its entirety. Production of pseudotyped AAV viral particles is disclosed in, for example, WO 2018/022608 and WO 2001/83692, each of which is herein incorporated by reference in its entirety. Other types of AAV viral particle variants, for example AAV viral particles with capsid mutations, are also contemplated. See, for example, Marsic et al., Molecular Therapy, 22(11): 1900-1909 (2014), which is herein incorporated by reference its entirety. For example, without limitation, the ITRs and/or the Rep proteins may be of, for example, the capsid proteins are derived from sequences of AAV found in a mammal such as, for example, capsid sequences disclosed and designated herein as Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, or Bfm35 or variants thereof.

As used herein, an “AAV vector genome”, “vector genome”, or “rAAV vector genome” refers to single-stranded nucleic acids. An rAAV viral particle has an rAAV vector genome encapsidated within a capsid. The rAAV vector genome has an AAV 5′ inverted terminal repeat (ITR) sequence and an AAV 3′ ITR flanking a protein-coding sequence (preferably a functional therapeutic protein-encoding sequence; e.g., FVIII, FIX, and PAH) operably linked to transcription regulatory elements that are heterologous to the AAV viral genome, e.g., one or more promoters and/or enhancers and, optionally, a polyadenylation sequence and/or one or more introns inserted in the regulatory elements or between the regulatory elements and the protein-coding sequence or between exons of the protein-coding sequence. rAAV vector genome refers to nucleic acids that are present in the rAAV virus particle and can be either the sense strand or the anti-sense strand of the nucleic acid sequences disclosed herein. The size of such single-stranded nucleic acids is provided in bases. The terms “inverted terminal repeat” and “ITR” as used herein refers to the art-recognized regions found at the 5′ and 3′ termini of the rAAV genome which function in cis as origins of viral DNA replication and as packaging signals for the viral genome. AAV ITRs, together with the Rep proteins, provide for efficient excision and rescue from, and integration of a nucleotide sequence interposed between two flanking ITRs into a host cell genome. Sequences of certain AAV-associated ITRs are disclosed by Yan et al., J. Virol. 79(1):364-379 (2005). ITRs are also found in a “flip” or “flop” configuration in which the sequence between the AA′ inverted repeats (that form the arms of the hairpin) are present in the reverse complement (Wilmott, Patrick, et al. Human gene therapy methods 30.6 (2019): 206-213). Construction and use of AAV vector genomes of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001. Because of wide construct availability and extensive characterization, illustrative AAV vector genomes disclosed below are derived from serotype 2.

The terms “therapeutically effective AAV”, “therapeutically effective AAV particle”, “therapeutic AAV”, “therapeutically effective rAAV”, “therapeutically effective rAAV particle”, “therapeutic rAAV”, and “therapeutically effective rAAV” refer to recombinant AAV that are capable of infecting cells such that the infected cells express (e.g., by transcription and/or by translation) an element (e.g., nucleotide sequence, protein, etc.) of interest. To this extent, the therapeutically effective rAAV particles can include AAV particles having capsids or vector genomes (vgs) with different properties. For example, the therapeutically effective rAAV particles can have capsids with different post translation modifications. In other examples, the therapeutically effective AAV particles can contain vgs with differing sizes/lengths, plus or minus strand sequences, different flip/flop ITR configurations flip/flop, flop/flip, flip/flip, flop/flop, etc.), different number of ITRs (1, 2, 3, etc.), or truncations. For example, overlapping homologous recombination occurs in rAAV infected cells between nucleic acids having 5′ end truncations and 3′ end truncations so that a “complete” nucleic acid encoding the large protein is generated, thereby reconstructing a functional, full-length gene. In other examples, complementary nucleic acid sequences having 5′ end truncations and 3′ end truncations interact with each such that a “complete” nucleic acid is formed during second strand synthesis. The “complete” nucleic acid encodes the large protein, thereby reconstructing a functional, full-length gene. Therapeutically effective rAAV particles are also referred to as heavy capsids, full capsids, or partially full capsids.

The terms “transduction” and “transduce” refers to the transfer of genetic material (e.g., vector genome) from an rAAV into a recipient cell and the expression transgene from the rAAV genetic material in the recipient cell. The transfer of the genetic material is mediated through an rAAV particle infecting a recipient cell. To this end, the term “potency” refers to the level of transgene expression in a recipient cell or recipient cells infected by rAAV particles. Thus, an rAAV having a greater potency highlights that a recipient cell infected by rAAV has greater transgene expression.

The term “therapeutically effective amount” means an amount of a therapeutic agent that is sufficient, when administered to a subject suffering from or susceptible to a disease, disorder, or condition, to treat, diagnose, prevent, or delay the onset of the symptom(s) of the disease, disorder, or condition. It will be appreciated by those of ordinary skill in the art that a therapeutically effective amount is typically administered via a dosing regimen comprising at least one unit dose. The term “therapeutically effective” refers to any element or composition of a therapeutic agent acting sufficiently such that a therapeutically effective amount of the therapeutic agent is sufficient, when administered to a subject suffering from or susceptible to a disease, disorder, and/or condition, to treat, diagnose, prevent, and/or delay the onset of the symptom(s) of the disease, disorder, and/or condition. For example as previously noted, a therapeutically effective rAAV is capable of infecting cells such that the infected cells express (e.g., by transcription and/or by translation) an element (e.g., nucleotide sequence, protein, etc.) of interest. The therapeutically effective rAAV has a vector genome that is used by cells infected by the therapeutically effective rAAV to generate therapeutically effective nucleotide sequences that are used by the infected cell to generate an element (e.g., nucleotide sequence, protein, etc.) of interest by various methods such as replication, transcription, or translation. It is also noted that a “therapeutic agent” includes therapeutically effective rAAV or a therapeutic rAAV virus.

As an example, a “therapeutic rAAV virus”, which refers to an rAAV virion, rAAV viral particle, rAAV vector particle, or rAAV virus that comprises a heterologous polynucleotide that encodes a therapeutic protein, can be used to replace or supplement the protein in vivo. The “therapeutic protein” is a polypeptide that has a biological activity that replaces or compensates for the loss or reduction of activity of a corresponding endogenous protein. For example, a functional phenylalanine hydroxylase (PAH) is a therapeutic protein for phenylketonuria (PKU). Thus, for example recombinant rAAV PAH virus can be used for a medicament for the treatment of a subject suffering from PKU. The medicament may be administered by intravenous (IV) administration and the administration of the medicament results in expression of PAH protein in the bloodstream of the subject sufficient to alter the neurotransmitter metabolite or neurotransmitter levels in the subject. Optionally, the medicament may also comprise a prophylactic and/or therapeutic corticosteroid for the prevention and/or treatment of any hepatotoxicity associated with administration of the rAAV PAH virus. The medicament comprising a prophylactic or therapeutic corticosteroid treatment may comprise at least 5, 10, 15, 20, 25, 30, 35, 40, 45, 50, 55, 60, or more mg/day of the corticosteroid. The medicament comprising a prophylactic or therapeutic corticosteroid may be administered over a continuous period of at least about 3, 4, 5, 6, 7, 8, 9, 10 weeks, or more. The PKU therapy may optionally also include tyrosine supplements.

The transgene incorporated into the AAV capsid is not limited and may be any heterologous gene of therapeutic interest. The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a host cell.

The composition of the transgene sequence will depend upon the use to which the resulting vector will be put. For example, one type of transgene sequence includes a reporter sequence, which upon expression produces a detectable signal. Such reporter sequences include, without limitation, DNA sequences encoding b-lactamase, b-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (MA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

However, the transgene is typically a non-marker sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated animal. Typically, suitable target sequences include oncologic targets and viral diseases. See for examples of such targets the oncologic targets and viruses identified below in the section relating to immunogens.

The transgene may be used to correct or ameliorate gene deficiencies, which may include deficiencies in which normal genes are expressed at less than normal levels or deficiencies in which the functional gene product is not expressed. A preferred type of transgene sequence encodes a therapeutic protein or polypeptide which is expressed in a host cell. The vector may further include multiple transgenes, e.g., to correct or ameliorate a gene defect caused by a multi-subunit protein. In certain situations, a different transgene may be used to encode each subunit of a protein, or to encode different peptides or proteins. This is desirable when the size of the DNA encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same transgene. In this case, a single transgene includes the DNA encoding each of the subunits, with the DNA for each subunit separated by an internal ribozyme entry site (IRES). This is desirable when the size of the DNA encoding each of the subunits is small, e.g., the total size of the DNA encoding the subunits and the IRES is less than five kilobases (Kb). It is also noted that longer genomes (i.e., >5 (Kb)) might be feasible due to recombination of partial genomes in target cells. As an alternative to an IRES, the DNA may be separated by sequences encoding a 2A peptide, which self-cleaves in a post-translational event. See, e.g., Donnelly et al, J. Gen. Virol., 78(Pt 1): 13-21 (January 1997); Furler, et al, Gene Ther., 8(1 1):864-873 (June 2001); Klump et al, Gene Ther., 8(1O):8 11-817 (May 2001). This 2A peptide is significantly smaller than an IRES, making it well suited for use when space is a limiting factor. More often, when the transgene is large, consists of multi-subunits, or two transgenes are co-delivered, rAAV carrying the desired transgene(s) or subunits are co-administered to allow them to concatamerize in vivo to form a single vector genome. In such an embodiment, a first AAV may carry an expression cassette which expresses a single transgene and a second AAV may carry an expression cassette which expresses a different transgene for co-expression in the host cell. However, the selected transgene may encode any biologically active product or other product, e.g., a product desirable for study.

Suitable transgenes may be readily selected by one of skill in the art. The selection of the transgene is not considered to be a limitation of this invention. The transgene may be a heterologous protein, and this heterologous protein may be a therapeutic protein. Exemplary therapeutic proteins include, but are not limited to, blood factors, such as b-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a.), transforming growth factor beta (TGF-.b.), and the like; soluble receptors, such as soluble TNF-a. receptors, soluble VEGF receptors, soluble interleukin receptors (e.g., soluble IL-1 receptors and soluble type II IL-1 receptors), soluble g/d T cell receptors, ligand-binding fragments of a soluble receptor, and the like; enzymes, such as a-glucosidase, imiglucarase, b-glucocerebrosidase, and alglucerase; enzyme activators, such as tissue plasminogen activator; chemokines, such as 1P-10, monokine induced by interferon-gamma (Mig), Groa/IL-8, RANTES, MIP-1a, MIR-1b., MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors, luteinizing hormone; macrophage activating factors; tumor necrosis factor (TNF); neutrophil chemotactic factor (NCF); tissue inhibitors of metalloproteinases; vasoactive intestinal peptide; angiogenin; angiotropin; fibrin; hirudin; IF-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; hereditary angioedema related proteins such as Cl-inhibitor; dystrophin, mini-dystrophin, or microdystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GFUT2), aldolase A, b-enolase, and glycogen synthase; lysosomal enzymes (e.g., beta-N-acetylhexosaminidase A); and any variants thereof. Other transgenes include transgenes encoding cardiac myosin binding protein C, β-myosin heavy chain, cardiac troponin T, cardiac troponin I, myosin ventricular essential light chain 1, myosin ventricular regulatory light chain 2, cardiac α actin (ACTC), α-tropomyosin, titin, four-and-a-half LIM protein 1, and other transgenes disclosed in U.S. Patent No. in International Application Publication No. WO 2014/170470. The AAV vector also includes conventional control elements or sequences which are operably linked to the transgene in a manner which permits its transcription, translation and/or expression in a cell transfected with the plasmid vector or infected with the virus. As used herein, “operably linked” sequences include both expression control sequences that are contiguous with the gene of interest (GOI) and expression control sequences that act in trans or at a distance to control the gene of interest. Suitable genes include those genes discussed in Anguela et al. “Entering the Modern Era of Gene Therapy”, Annual Rev. of Med. Vol. 70, pages 272-288 (2019) and Dunbar et al., “Gene Comes of Age”, Science, Vol. 359, Issue 6372, eaan4672 (2018).

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart el al, Cell, 41:521-530 (1985)), the SV40 promoter, the dihydrofolate reductase promoter, the b-actin promoter, the phosphoglycerol kinase (PGK) promoter, and the EF1 promoter [Invitrogen]. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied compounds, include, the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracyclinerepressible system [Gossen et al., Proc. Natl. Acad. Sci. USA, 89:5547-5551 (1992)], the tetracycline-inducible system [Gossen et al., Science, 268:1766-1769 (1995), see also Harvey et al., Curr. Opin. Chem. Biol., 2:512-518 (1998)], the RU486-inducible system [Wang et al., Nat. Biotech., 15:239-243 (1997) and Wang et al., Gene Ther., 4:432-441 (1997)] and the rapamycininducible system [Magari et al., J. Clin. Invest., 100:2865-2872 (1997)]. Other types of inducible promoters which may be useful in this context are those which are regulated by a specific physiological state, e.g., temperature, acute phase, a particular differentiation state of the cell, or in replicating cells only.

Optionally, the native promoter for the transgene may be used. The native promoter may be preferred when it is desired that expression of the transgene should mimic the native expression. The native promoter may be used when expression of the transgene is preferably regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

The transgene may also include a gene operably linked to a tissue specific promoter. For instance, if expression in skeletal muscle is desired, a promoter active in muscle should be used. These include the promoters from genes encoding skeletal b-actin, myosin light chain 2A, dystrophin, muscle creatine kinase, as well as synthetic muscle promoters with activities higher than naturally-occurring promoters (see Li et al., Nat. Biotech., 17:241-245 (1999)). Examples of promoters that are tissue-specific are known for liver (albumin, Miyatake et al., J. Virol., 71:5124-32 (1997); hepatitis B virus core promoter, Sandig et al., Gene Ther., 3:1002-9 (1996); alpha-fetoprotein (AFP), Arbuthnot et al., Hum. Gene Ther., 7:1503-14 (1996)), bone osteocalcin (Stein et al., Mol. Biol. Rep., 24:185-96 (1997)); bone sialoprotein (Chen et al., J. Bone Miner. Res., 11:654-64 (1996)), lymphocytes (CD2, Hansal et al., J. Immunol., 161:1063-8 (1998); immunoglobulin heavy chain; T cell receptor chain), neuronal such as neuron-specific enolase (NSE) promoter (Andersen et al., Cell. Mol. Neurobiol., 13:503-15 (1993)), neurofilament light-chain gene (Piccioli et al., Proc. Natl. Acad. Sci. USA, 88:5611-5 (1991)), and the neuron-specific vgf gene (Piccioli et al., Neuron, 15:373-84 (1995)), among others.

The recombinant AAV can be used to produce a protein of interest in vitro, for example, in a cell culture. For example, the AAV can be used in a method for producing a protein of interest in vitro, where the method includes providing a recombinant AAV comprising a nucleotide sequence encoding the heterologous protein; and contacting the recombinant AAV with a cell in a cell culture, whereby the recombinant AAV expresses the protein of interest in the cell. The size of the nucleotide sequence encoding the protein of interest can vary. For example, the nucleotide sequence can be at least about 0.1 kilobases (kb), at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

The recombinant AAV can also be used to produce a protein of interest in vivo, for example in an animal such as a mammal. Some embodiments provide a method for producing a protein of interest in vivo, where the method includes providing a recombinant AAV comprising a nucleotide sequence encoding the protein of interest; and administering the recombinant AAV to the subject, whereby the recombinant AAV expresses the protein of interest in the subject. The subject can be, in some embodiments, a non-human mammal, for example, a monkey, a dog, a cat, a mouse, or a cow. The size of the nucleotide sequence encoding the protein of interest can vary. For example, the nucleotide sequence can be at least about 0.1 kb, at least about 0.2 kb, at least about 0.3 kb, at least about 0.4 kb, at least about 0.5 kb, at least about 0.6 kb, at least about 0.7 kb, at least about 0.8 kb, at least about 0.9 kb, at least about 1 kb, at least about 1.1 kb, at least about 1.2 kb, at least about 1.3 kb, at least about 1.4 kb, at least about 1.5 kb, at least about 1.6 kb, at least about 1.7 kb, at least about 1.8 kb, at least about 2.0 kb, at least about 2.2 kb, at least about 2.4 kb, at least about 2.6 kb, at least about 2.8 kb, at least about 3.0 kb, at least about 3.2 kb, at least about 3.4 kb, at least about 3.5 kb in length, at least about 4.0 kb in length, at least about 5.0 kb in length, at least about 6.0 kb in length, at least about 7.0 kb in length, at least about 8.0 kb in length, at least about 9.0 kb in length, or at least about 10.0 kb in length. In some embodiments, the nucleotide is at least about 1.4 kb in length.

Of particular interest is the use of recombinant AAV to express one or more therapeutic proteins to treat various diseases or disorders. Non-limiting examples of the diseases include cancer such as carcinoma, sarcoma, leukemia, lymphoma; and autoimmune diseases such as multiple sclerosis. Non-limiting examples of carcinomas include esophageal carcinoma; hepatocellular carcinoma; basal cell carcinoma, squamous cell carcinoma (various tissues); bladder carcinoma, including transitional cell carcinoma; bronchogenic carcinoma; colon carcinoma; colorectal carcinoma; gastric carcinoma; lung carcinoma, including small cell carcinoma and non-small cell carcinoma of the lung; adrenocortical carcinoma; thyroid carcinoma; pancreatic carcinoma; breast carcinoma; ovarian carcinoma; prostate carcinoma; adenocarcinoma; sweat gland carcinoma; sebaceous gland carcinoma; papillary carcinoma; papillary adenocarcinoma; cystadenocarcinoma; medullary carcinoma; renal cell carcinoma; ductal carcinoma in situ or bile duct carcinoma; choriocarcinoma; seminoma; embryonal carcinoma; Wilm's tumor; cervical carcinoma; uterine carcinoma; testicular carcinoma; osteogenic carcinoma; epithelieal carcinoma; and nasopharyngeal carcinoma. Non-limiting examples of sarcomas include fibrosarcoma, myxosarcoma, liposarcoma, chondrosarcoma, chordoma, osteogenic sarcoma, osteosarcoma, angiosarcoma, endothelio sarcoma, lymphangiosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Non-limiting examples of leukemias include chronic myeloproliferative syndromes; acute myelogenous leukemias; chronic lymphocytic leukemias, including B-cell CLL, T-cell CLL prolymphocytic leukemia, and hairy cell leukemia; and acute lymphoblastic leukemias. Examples of lymphomas include, but are not limited to, B-cell lymphomas, such as Burkitt's lymphoma; Hodgkin's lymphoma; and the like.

Other non-liming examples of the diseases that can be treated using rAAV and methods disclosed herein include genetic disorders including sickle cell anemia, cystic fibrosis, lysosomal acid lipase (LAL) deficiency 1, Tay-Sachs disease, Phenylketonuria, Mucopolysaccharidoses, Glycogen storage diseases (GSD, e.g., GSD types I, II, III, IV, V, VI, VII, VIII, IX, X, XI, XII, XIII, and XIV), Galactosemia, muscular dystrophy (e.g., Duchenne muscular dystrophy), cardiomyopathies (e.g., hypertrophic cardiomyopathy, dilated cardiomyopathy, arrhythmogenic right ventricular cardiomyopathy, etc.) and hemophilia such as hemophilia A (classic hemophilia) and hemophilia B (Christmas Disease), Wilson's disease, Fabry Disease, Gaucher Disease hereditary angioedema (HAE), and alpha 1 antitrypsin deficiency. In addition, the rAAV and methods disclosed herein can be used to treat other disorders that can be treated by local expression of a transgene in the liver or by expression of a secreted protein from the liver or a hepatocyte.

The amount of the heterologous protein expressed in the subject (e.g., the serum of the subject) can vary. For example, in some embodiments the protein can be expressed in the serum of the subject in the amount of at least about 9 milligram (mg)/mL, at least about 10 mg/mL, at least about 11 mg/mL, at least about 12 mg/mL, at least about 13 mg/mL, at least about 14 mg/mL, at least about 15 mg/mL, at least about 16 mg/mL, at least about 17 mg/mL, at least about 18 mg/mL, at least about 19 mg/mL, at least about 20 mg/mL, at least about 21 mg/mL, at least about 22 mg/mL, at least about 23 mg/mL, at least about 24 mg/mL, at least about 25 mg/mL, at least about 26 mg/mL, at least about 27 mg/mL, at least about 28 mg/mL, at least about 29 mg/mL, at least about 30 mg/mL, at least about 31 mg/mL, at least about 32 mg/mL, at least about 33 mg/mL, at least about 34 mg/mL, at least about 35 mg/mL, at least about 36 mg/mL, at least about 37 mg/mL, at least about 38 mg/mL, at least about 39 mg/mL, at least about 40 mg/mL, at least about 41 mg/mL, at least about 42 mg/mL, at least about 43 mg/mL, at least about 44 mg/mL, at least about 45 mg/mL, at least about 46 mg/mL, at least about 47 mg/mL, at least about 48 mg/mL, at least about 49 mg/mL, or at least about 50 mg/mL. The protein of interest may be expressed in the serum of the subject in the amount of about 9 pg/mL, about 10 pg/mL, about 50 pg/mL, about 100 pg/mL, about 200 pg/mL, about 300 pg/mL, about 400 pg/mL, about 500 pg/mL, about 600 pg/mL, about 700 pg/mL, about 800 pg/mL, about 900 pg/mL, about 1000 pg/mL, about 1500 pg/mL, about 2000 pg/mL, about 2500 pg/mL, or a range between any two of these values. A skilled artisan will understand that the expression level in which a protein of interest is needed for therapeutic efficacy can vary depending on non-limiting factors, such as the particular protein of interest and the subject receiving the treatment, and an effective amount of the protein can be readily determined by a skilled artisan using conventional methods known in the art without undue experimentation.

In other embodiments, the present invention is directed to pharmaceutical formulations of AAV viral particles of the present invention useful for administration to a subject.

In one embodiment, the pharmaceutical formulations of the present invention are liquid formulations that comprise AAV viral particles disclosed herein, wherein the concentration of AAV viral particles in the formulation may vary widely. AAV viral particles and compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the amount of AAV viral particles necessary to produce the desired effect(s). The amount necessary can be formulated in a single dose or can be formulated in multiple dosage units. The dose may be adjusted to a suitable AAV viral particle concentration, optionally combined with one or more other agents, and packaged for use.

In another embodiment, pharmaceutical compositions will include sufficient genetic material to provide a prophylactically or therapeutically effective amount, i.e., an amount sufficient to prevent, reduce or ameliorate symptoms of a disease state in question or an amount sufficient to confer the desired benefit.

In other embodiments, the AAV viral particle containing pharmaceutical formulation of the invention comprises one or more pharmaceutically acceptable excipients to provide the formulation with advantageous properties for storage and/or administration to subjects. In certain embodiments, the pharmaceutical formulations of the present invention are capable of being stored at <65° C. for a period of at least 2 weeks, preferably at least 4 weeks, more preferably at least 6 weeks and yet more preferably at least about 8 weeks, without detectable change in stability. In this regard, the term “stable” means that the AAV viral particles present in the formulation essentially retains its physical stability, chemical stability and/or biological activity during storage. In other embodiments of the present invention, the AAV viral particle present in the pharmaceutical formulation retains at least about 80% of its biological activity in a subject during storage for a determined period of time at −65° C., more preferably at least about 85%, 90%, 95%, 98% or 99% of its biological activity in a subject.

In some embodiments, sodium phosphate dibasic at a concentration of about 0.1 mg/ml to about 3 mg/ml, about 0.5 mg/ml to about 2.5 mg/ml, about 1 mg/ml to about 2 mg/ml, or about 1.4 mg/ml to about 1.6 mg/ml. In a particularly preferred embodiment, the AAV viral particle formulation of the present invention comprises about 1.42 mg/ml of sodium phosphate, dibasic (dried).

In other embodiments, another buffering agent that may find use in the AAV viral particle formulations of the present invention is sodium phosphate, monobasic monohydrate which, in some embodiments, finds use at a concentration of from about 0.1 mg/ml to about 3 mg/ml, about 0.5 mg/ml to about 2.5 mg/ml, about 1 mg/ml to about 2 mg/ml, or about 1.3 mg/ml to about 1.5 mg/ml. In one embodiment, the AAV viral particle formulation of the present invention comprises about 1.38 mg/ml of sodium phosphate, monobasic monohydrate. In another embodiment, the AAV viral particle formulation of the present invention comprises about 1.42 mg/ml of sodium phosphate, dibasic and about 1.38 mg/ml of sodium phosphate, monobasic monohydrate.

In one embodiment, the AAV viral particle formulation of the present invention may comprise one or more isotonicity agents, such as sodium chloride, preferably at a concentration of about 1 mg/ml to about 20 mg/ml, for example, about 1 mg/ml to about 10 mg/ml, about 5 mg/ml to about 15 mg/ml, or about 8 mg/ml to about 20 mg/ml. In another embodiment, the formulation of the present invention comprises about 8.18 mg/ml sodium chloride. Other buffering agents and isotonicity agents known in the art are suitable and may be routinely employed for use in the formulations of the present disclosure.

In another embodiment, the AAV viral particle formulations of the present invention may comprise one or more bulking agents. Exemplary bulking agents include without limitation mannitol, sucrose, dextran, lactose, trehalose, and povidone (PVP K24). In certain preferred embodiments, the formulations of the present invention comprise mannitol, which may be present in an amount from about 5 mg/ml to about 40 mg/ml, or from about 10 mg/ml to about 30 mg/ml, or from about 15 mg/ml to about 25 mg/ml. In a particularly preferred embodiment, mannitol is present at a concentration of about 20 mg/ml.

In some embodiment, the AAV viral particle formulations of the present invention may comprise one or more surfactants, which may be non-ionic surfactants.

Exemplary surfactants include, but are not limited to, ionic surfactants, non-ionic surfactants, and combinations thereof. For example, the surfactant can be, without limitation, TWEEN 80 (also known as polysorbate 80, or its chemical name polyoxyethylene sorbitan monooleate), sodium dodecylsulfate, sodium stearate, ammonium lauryl sulfate, TRITON AG 98 (Rhone-Poulenc), poloxamer 407, poloxamer 188 and the like, and combinations thereof.

In some embodiments, the formulation of the present invention comprises poloxamer 188, which may be present at a concentration of from about 0.1 mg/ml to about 4 mg/ml, or from about 0.5 mg/ml to about 3 mg/ml, from about 1 mg/ml to about 3 mg/ml, about 1.5 mg/ml to about 2.5 mg/ml, or from about 1.8 mg/ml to about 2.2 mg/ml. In a particularly preferred embodiment, poloxamer 188 is present at a concentration of about 2.0 mg/ml.

In other embodiments, the pharmaceutical formulation of the present invention comprises AAV viral particle formulated in a liquid solution that comprises about 1.42 mg/ml of sodium phosphate, dibasic, about 1.38 mg/ml of sodium phosphate, monobasic monohydrate, about 8.18 mg/ml sodium chloride, about 20 mg/ml mannitol and about 2 mg/ml poloxamer 188.

In some embodiments, the AAV viral particle-containing formulations of the present disclosure are stable and can be stored for extended periods of time without an unacceptable change in quality, potency, or purity.

In one embodiment, the formulation is stable at a temperature of about 5° C. (e.g., 2° C. to 8° C.) for at least 1 month, for example, at least 1 month, at least 3 months, at least 6 months, at least 12 months, at least 18 months, at least 24 months, or more.

In another embodiment, the formulation is stable at a temperature of less than or equal to about −20° C. for at least 6 months, for example, at least 6 months, at least 12 months, at least 18 months, at least 24 months, at least 36 months, or more.

In some embodiments, the formulation is stable at a temperature of less than or equal to about −40° C. for at least 6 months, for example, at least 6 months, at least 12 months, at least 18 months, at least 24 months, at least 36 months, or more.

In other embodiments, the formulation is stable at a temperature of less than or equal to about −60° C. for at least 6 months, for example, at least 6 months, at least 12 months, at least 18 months, at least 24 months, at least 36 months, or more.

In one embodiment, the present invention provides uses of the AAV viral particles of the invention for efficient transduction of cells, tissues, and/or organs of interest, and/or for use in gene therapy.

In one embodiment, the present invention provides a method for transduction of cells, tissues, and/or organs of interest, comprising introducing into a cell, a composition comprising an effective amount of the AAV viral particles of the present invention.

In some embodiments, AAV viral particles of the invention are used for transduction of cells, tissues, and/or organs of interest of a subject.

In other embodiments, a method for transduction of cells, tissues, and/or organs of interest, comprising introducing into a cell is provided, the method comprising a composition comprising an effective amount of AAV viral particles of the present invention.

In one embodiment, methods for prophylactic or therapeutic treatment of a subject are provided.

In another embodiment, the subject is need thereof of the prophylactic or therapeutic treatment.

In some embodiments, the subject comprises a condition or disease, wherein the subject is need of treatment for said condition or disease.

In other embodiments, the subject is a mammal.

In other embodiments, the subject is a non-rodent mammal, primate, human, livestock, horse, sheep, goat, pig, dog, or cat.

In another embodiment, AAV viral particles of the present invention may be administered to the subject through a variety of known administration techniques.

In some embodiments, the AAV viral particle is administered by intravenous injection either as a single bolus or over a prolonged time period, which may be at least about 1, 5, 10, 15, 30, 45, 60, 75, 90, 120, 150, 180, 210 or 240 minutes, or more.

In one embodiment, cells (e.g., ependymal cells) comprising the cerebrospinal fluid (CSF) of the subject are transduced by said AAV viral particles. In some embodiments, cells (e.g., ependymal cells) transduced with the AAV viral particles express and secrete the transgene(s) into the CSF of said mammal.

In another embodiment, administration of the AAV viral particles comprises administration to the cisterna magna, intraventricular space, brain ventricle, subarachnoid space, intrathecal space and/or ependyma of the subject.

In other embodiments, administration of the AAV viral particles comprises administration to the cerebral spinal fluid (CSF) of said subject.

In some embodiments, administration of the AAV viral particles comprises contacting ependymal cells of said subject with the AAV viral particles.

In one embodiment, administration of the AAV viral particles comprises contacting a pial cell, endothelial cell, or meningeal cell of said subject with said AAV viral particles.

In another embodiment, administration of the AAV viral particles comprises injection of the AAV viral particles into a tissue or fluid of the brain or spinal cord of said subject.

In some embodiments, administration of the AAV viral particles comprises injection of the AAV viral particles into cerebral spinal fluid of said subject.

In another embodiment, the present invention provides a kit for use with methods and compositions described herein. Compositions and virus formulations may be provided in the kit. The kits can also include a suitable container and optionally one or more additional agents. In some embodiments, the container is a vial, test tube, flask, bottle, syringe and/or other container. In other embodiments, the kit comprises the AAV viral particle, a pharmaceutically acceptable carrier, and instructional material for the use thereof, for example, for directing the administration of the AAV viral particle.

### Methods of Producing Adeno Associated Virus

The present disclosure provides materials and methods for producing rAAV virions in cells such as mammalian and insect cells.

In various embodiments, the method of any embodiment includes the step culturing a host cell having one or more vectors for rAAV production. In other embodiments, the one or more vectors for rAAV production includes at least one nucleic acid molecule that provides AAV helper function, or at least one nucleic acid molecule that provides non-AAV helper function, or at least one nucleic acid molecule that generates an AAV genome vector, or any combination thereof. The method of any embodiment further includes culturing the cells under conditions that that permit production of the rAAV. The method optionally includes recovering the rAAV. For example, the AAV viral particles can be collected at about 12 hours, about 24 hours, about 36 hours, about 48 hours, about 72 hours, about 96 hours, about 120 hours, about 144 hours, about 168 hours, about 192 hours, about 216 hours, about 240 hours, or a time between any of these two time points after the co-transfection.

In some embodiments, cultures for the production of AAV viral particle comprise one or more of the following: the host cell, a suitable helper virus function, provided by wild type or mutant adenovirus (such as temperature sensitive adenovirus), herpes virus, baculovirus, or a plasmid construct providing helper functions, an AAV rep and cap genes and gene products, a transgene (such as diagnostic and/or therapeutic transgene(s)) flanked by AAV ITR sequences, and suitable media and media components to support AAV viral particle production.

In some embodiments, the insect or mammalian cell can be transfected with the helper plasmid or helper virus, the viral construct and the plasmid encoding the AAV cap genes; and the rAAV particles can be collected at various time points after co-transfection.

In some embodiments, a novel rAAV viral particle is produced in insect cells (e.g., Sf9). In some embodiments, an AAV viral particle is prepared by providing to a host cell with an AAV genome vector comprising a transgene together with a Rep and Cap gene. In some embodiments, an AAV genome vector comprises a transgene, an AAV Rep gene and an AAV Cap gene. In some embodiments, an rAAV viral particle is prepared by providing to a host cell with two or more vectors. For example, in some embodiments, an AAV genome vector comprising a transgene is introduced (e.g., transfected or transduced) into a cell with a vector (e.g., a plasmid or baculovirus) comprising an AAV Rep gene and a AAV Cap gene. In some embodiments, a cell transfected or transduced with an AAV genome vector comprising a transgene, a vector (e.g., a plasmid or baculovirus) comprising an AAV Rep gene, and a vector (e.g., a plasmid or baculovirus) comprising an AAV Cap gene.

In various embodiments, the method of any embodiment includes the steps of infecting the host cells with rBV. The rBV includes one or more nucleic acid molecules encoding Rep proteins, one or more nucleic acid molecules encoding capsid proteins, and at least one nucleic acid molecule that generates an AAV genome vector. The method of any embodiment further includes culturing the cells under conditions that that permit production of the rAAV. The method optionally includes recovering the rAAV.

There are a number of methods for generating AAV viral particles: for example, but not limited to, transfection using vector and AAV helper sequences in conjunction with coinfection with one of the AAV helper viruses (e.g., adenovirus, herpesvirus, or vaccinia virus) or transfection with a recombinant AAV vector, an AAV helper vector, and an accessory function vector. Methods of making AAV viral particles are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, WO2001023597, WO2015191508, WO2019217513, WO2018022608, WO2019222136, WO2020232044, WO2019222132; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir.63:3822-8 (1989); Kajigaya et al., Proc. Nat'l. Acad. Sci. USA 88: 4646-50 (1991); Ruffing et al., J. Vir.66:6922-30 (1992); Kimbauer et al., Vir., 219:37-44 (1996); Zhao et al., Vir.272:382-93 (2000); the contents of each of which are herein incorporated by reference in their entirety. For detailed descriptions of methods for generating AAV viral particles see, for example, U.S. Pat. Nos. 6,001,650, 6,004,797, and 9,504,762, each herein incorporated by reference in its entirety. In one embodiment, a triple transfection method (see, e.g., U.S. Pat. No. 6,001,650, herein incorporated by reference in its entirety) is used to produce AAV viral particles. This method does not require the use of an infectious helper virus, enabling AAV viral particles to be produced without any detectable helper virus present. This is accomplished by use of three vectors for AAV viral particle production, namely an AAV helper function vector, an accessory function vector, and an AAV viral particle expression vector. One of skill in the art will appreciate, however, that the nucleic acid sequences encoded by these vectors can be provided on two or more vectors in various combinations. In other embodiments, the host cell can be transfected with the helper plasmid or helper virus, the viral construct and the plasmid encoding the AAV cap genes; and the AAV viral particles can be collected at various time points after co-transfection.

For example, wild-type AAV and helper viruses may be used to provide the necessary replicative functions for producing AAV viral particles (see, e.g., U.S. Pat. No. 5,139,941, herein incorporated by reference in its entirety). Alternatively, a plasmid, containing helper function genes, in combination with infection by one of the well-known helper viruses can be used as the source of replicative functions (see e.g., U.S. Pat. Nos. 5,622,856 and 5,139,941, both herein incorporated by reference in their entireties). Similarly, a plasmid, containing accessory function genes can be used in combination with infection by wild-type AAV, to provide the necessary replicative functions. Other approaches, described herein and/or well known in the art, can also be employed by the skilled artisan to produce AAV viral particles.

In various embodiments, the culturing step of any embodiment occurs in a volume of at least 20 milliliter(s) (mL), at least 50 mL, at least 100 mL, at least 500 mL, at least 1 liter (L), at least 10 L, at least 50 L, at least 100 L, at least 250 L, at least 500 L, at least 1000 L, at least 1500 L, at least 2000 L, or at least 2500 L.

In examples, the culturing step can occur in a shake flask or shake flasks. In various embodiments, the culturing step of any embodiment occurs in a volume of 20 mL, 30 mL, 40 mL, 50 mL, 60 mL, 70 mL, 80 mL, 90 mL, 100 mL, 200 mL, 300 mL, 400 mL, 500 mL, 600 mL, 700 mL, 800 mL, 900 mL, 1 L, 2 L, 3 L, 4 L, or 5 L. In other embodiments, the volume of the culturing step is a range between any two volumes provided above.

In other examples, the culturing step can occur in a bioreactor or bioreactors. In various embodiments, the culturing step of any embodiment occurs in a volume of 1 L, 2 L, 3 L, 4 L, 5 L, 6L, 7L, 8 L, 9 L, 10 L, 11 L, 12 L, 13 L, 14 L, 15 L, 16 L, 17 L, 18 L, 19 L, 20 L, 21 L, 22 L, 23 L, 24 L, 25 L, 26 L, 27 L, 28 L, 29 L, 30 L, 31 L, 32 L, 33 L, 34 L, 35 L, 36 L, 37 L, 38 L, 39 L, 40 L, 41 L, 42 L, 43 L, 44 L, 45 L, 46 L, 47 L, 48 L, 49 L, 50 L, 51 L, 52 L, 53 L, 54 L, 55 L, 56 L, 57 L, 58 L, 59 L, 60 L, 61 L, 62 L, 63 L, 64 L, 65 L, 66 L, 67 L, 68 L, 69 L, 70 L, 71 L, 72 L, 73 L, 74 L, 75 L, 76 L, 77 L, 78 L, 79 L, 80 L, 81 L, 82 L, 83 L, 84 L, 85 L, 86 L, 87 L, 88 L, 89 L, 90 L, 91 L, 92 L, 93 L, 94 L, 95 L, 96 L, 97 L, 98 L, 99 L, 100 L, 110 L, 120 L, 130 L, 140 L, 150 L, 160 L, 170 L, 180 L, 190 L, 200 L, 210 L, 220 L, 230 L, 240 L, 250 L, 260 L, 270 L, 280 L, 290 L, 300 L, 310 L, 320 L, 330 L, 340 L, 350 L, 360 L, 370 L, 380 L, 390 L, 400 L, 410 L, 420 L, 430 L, 440 L, 450 L, 460 L, 470 L, 480 L, 490 L, 500 L, 510 L, 520 L, 530 L, 540 L, 550 L, 560 L, 570 L, 580 L, 590 L, 600 L, 610 L, 620 L, 630 L, 640 L, 650 L, 660 L, 670 L, 680 L, 690 L, 700 L, 710 L, 720 L, 730 L, 740 L, 750 L, 760 L, 770 L, 780 L, 790 L, 800 L, 810 L, 820 L, 830 L, 840 L, 850 L, 860 L, 870 L, 880 L, 890 L, 900 L, 910 L, 920 L, 930 L, 940 L, 950 L, 960 L, 970 L, 980 L, 990 L, 1000 L, 1010 L, 1020 L, 1030 L, 1040 L, 1050 L, 1060 L, 1070 L, 1080 L, 1090 L, 1100 L, 1110 L, 1120 L, 1130 L, 1140 L, 1150 L, 1160 L, 1170 L, 1180 L, 1190 L, 1200 L, 1210 L, 1220 L, 1230 L, 1240 L, 1250 L, 1260 L, 1270 L, 1280 L, 1290 L, 1300 L, 1310 L, 1320 L, 1330 L, 1340 L, 1350 L, 1360 L, 1370 L, 1380 L, 1390 L, 1400 L, 1410 L, 1420 L, 1430 L, 1440 L, 1450 L, 1460 L, 1470 L, 1480 L, 1490 L, 1500 L, 1510 L, 1520 L, 1530 L, 1540 L, 1550 L, 1560 L, 1570 L, 1580 L, 1590 L, 1600 L, 1610 L, 1620 L, 1630 L, 1640 L, 1650 L, 1660 L, 1670 L, 1680 L, 1690 L, 1700 L, 1710 L, 1720 L, 1730 L, 1740 L, 1750 L, 1760 L, 1770 L, 1780 L, 1790 L, 1800 L, 1810 L, 1820 L, 1830 L, 1840 L, 1850 L, 1860 L, 1870 L, 1880 L, 1890 L, 1900 L, 1910 L, 1920 L, 1930 L, 1940 L, 1950 L, 1960 L, 1970 L, 1980 L, 1990 L, 2000 L, 2010 L, 2020 L, 2030 L, 2040 L, 2050 L, 2060 L, 2070 L, 2080 L, 2090 L, 2100 L, 2110 L, 2120 L, 2130 L, 2140 L, 2150 L, 2160 L, 2170 L, 2180 L, 2190 L, 2200 L, 2210 L, 2220 L, 2230 L, 2240 L, 2250 L, 2260 L, 2270 L, 2280 L, 2290 L, 2300 L, 2310 L, 2320 L, 2330 L, 2340 L, 2350 L, 2360 L, 2370 L, 2380 L, 2390 L, 2400 L, 2410 L, 2420 L, 2430 L, 2440 L, 2450 L, 2460 L, 2470 L, 2480 L, 2490 L, 2500 L, 2510 L, 2520 L, 2530 L, 2540 L, 2550 L, 2560 L, 2570 L, 2580 L, 2590 L, 2600 L, 2610 L, 2620 L, 2630 L, 2640 L, 2650 L, 2660 L, 2670 L, 2680 L, 2690 L, 2700 L, 2710 L, 2720 L, 2730 L, 2740 L, 2750 L, 2760 L, 2770 L, 2780 L, 2790 L, 2800 L, 2810 L, 2820 L, 2830 L, 2840 L, 2850 L, 2860 L, 2870 L, 2880 L, 2890 L, 2900 L, 2910 L, 2920 L, 2930 L, 2940 L, 2950 L, 2960 L, 2970 L, 2980 L, 2990 L, or 3000 L. In other embodiments, the volume of the culturing step is a range between any two volumes provided above.

The term “vector” is understood to refer to any genetic element, such as a plasmid, phage, transposon, cosmid, bacmid, mini-plasmid (e.g., plasmid devoid of bacterial elements), Doggybone DNA (e.g., minimal, closed-linear constructs), chromosome, virus, virion (e.g., baculovirus), etc., which is capable of replication when associated with the proper control elements and which can transfer gene sequences between cells. An “mammalian cell-compatible vector” or “vector” as used herein refers to a nucleic acid molecule capable of productive transformation or transfection of a mammal or mammalian cell. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. Vectors and methods for their use are described in the above cited references on molecular engineering of cells.

The vector from which the cell generates an rAAV vector genome may contain a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. The vector may also contain a posttranscriptional regulatory element downstream of the restriction site and upstream of the 3′ AAV ITR. The viral construct may further comprise a polynucleotide inserted at the restriction site and operably linked with the promoter, where the polynucleotide comprises the coding region of a protein of interest. In some embodiments, the viral construct further includes a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the viral construct further incudes a posttranscriptional regulatory element downstream of the restriction site and upstream of the 3′ AAV ITR. In some embodiments, the viral construct further includes a polynucleotide inserted at the restriction site and operably linked with the promoter, where the polynucleotide includes the coding region of a protein of interest. As a skilled artisan will appreciate, any one of the AAV vectors disclosed in the present application can be used in the method as the viral construct to produce the rAAV virions.

The term “AAV helper” refer to AAV-derived coding sequences which can be expressed to provide AAV gene products that, in turn, function in trans for productive AAV replication. Thus, AAV helper functions include both of the major AAV open reading frames (ORFs), rep and cap. The Rep expression products have been shown to possess many functions, including, among others: recognition, binding and nicking of the AAV origin of DNA replication; DNA helicase activity; and modulation of transcription from AAV (or other heterologous) promoters. The capsid (Cap) expression products supply necessary packaging functions. AAV helper functions are used herein to complement AAV functions in trans that are missing from AAV vector genomes.

For production, cells with AAV helper functions produce recombinant capsid proteins sufficient to form a capsid. This includes at least VP1 and VP3 proteins, but more typically, all three of VP1, VP2, and VP3 proteins, as found in native AAV. The sequence of the capsid proteins determines the serotype of the AAV virions produced by the host cell. Capsids useful in the invention include those derived from a number of AAV serotypes, including 1, 2, 3, 3B, 4, 5, 6, 7, 8, 9, 10, 11, 12, 13 or mixed serotypes (see, e.g., U.S. Pat. No. 8,318,480 for its disclosure of non-natural mixed serotypes). The capsid proteins can also be variants of natural VP1, VP2 and VP3, including mutated, chimeric or shuffled proteins. The capsid proteins can be those of rh.10 or other subtype within the various clades of AAV; various clades and subtypes are disclosed, for example, in U.S. Pat. No. 7,906,111. Because of wide construct availability and extensive characterization, illustrative AAV vectors disclosed below are derived from serotype 2. Construction and use of AAV vectors and AAV proteins of different serotypes are discussed in Chao et al., Mol. Ther. 2:619-623, 2000; Davidson et al., PNAS 97:3428-3432, 2000; Xiao et al., J. Virol. 72:2224-2232, 1998; Halbert et al., J. Virol. 74:1524-1532, 2000; Halbert et al., J. Virol. 75:6615-6624, 2001; and Auricchio et al., Hum. Molec. Genet. 10:3075-3081, 2001.

In various embodiments, nucleotide sequences encoding VP proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters such as the SV40 promoter, CMV promoter, RSV promoter, UBC promoter, EF1A promoter, PGK promoter, dihydrofolate reductase promoter, the b-actin promoter, TRE (Tet, Tet-On, Tet-Off) promoter, Cumate controlled systems (CuR/CuO) (See US2004/0205834), the temperature-induced HSP70 promoter, p5 promoter, p10 promoter, p19 promoter, and the p40 promoter. In another example, the nucleotide sequences can be operably linked to baculoviral promoters such as the polyhedrin (Polh) promoter, ΔIE1 promoter, p5 promoter, p10 promoter, p19 promoter, the p40 promoter, metallothionein promoter, 39K promoter, p6.9 promoter, and orf46 promoter.

For production, cells with AAV helper functions produce Rep proteins to promote production of rAAV. It has been found that infectious particles can be produced when at least one large Rep protein (Rep78 or Rep68) and at least one small Rep protein (Rep52 and Rep40) are expressed in cells. In a specific embodiment all four of Rep 78, Rep68, Rep52 and Rep 40 are expressed. Alternately, Rep78 and Rep52, Rep78 and Rep40, Rep 68 and Rep52, or Rep68 and Rep40 are expressed. Examples below demonstrate the use of the Rep78/Rep52 combination. Rep proteins can be derived from AAV-2 or other serotypes. In various embodiments, nucleotide sequences encoding Rep proteins can be operably linked to a suitable expression control sequence. In various embodiments, nucleotide sequences encoding Rep proteins can be operably linked to a suitable expression control sequence such as eukaryotic promoters. For example, the nucleotide sequences can be operably linked to eukaryotic promoters such as the SV40 promoter, CMV promoter, RSV promoter, UBC promoter, EF1A promoter, PGK promoter, dihydrofolate reductase promoter, the b-actin promoter, TRE (Tet, Tet-On, Tet-Off) promoter, Cumate controlled systems (CuR/CuO) (See US2004/0205834), and the temperature-induced HSP70 promoter, p5 promoter, p10 promoter, p19 promoter, and the p40 promoter. In other examples, the nucleotide sequences can be operably linked to baculoviral promoters such as the polyhedrin (Polh) promoter, 4IE1 promoter, p5 promoter, p10 promoter, p19 promoter, the p40 promoter, metallothionein promoter, 39K promoter, p6.9 promoter, and orf46 promoter.

In some embodiments, the AAV cap genes are present in a plasmid or bacmid. The plasmid can further include an AAV rep gene which may or may not correspond to the same serotype as the cap genes. The cap genes and/or rep gene from any AAV serotype.

Cells with AAV helper functions can also produce assembly-activating proteins (AAP), which help assemble capsids. In various embodiments, nucleotide sequences encoding AAP can be operably linked to a suitable expression control sequence. For example, the nucleotide sequences can be operably linked to eukaryotic promoters. In other examples, the nucleotide sequences can be operably linked to baculoviral promoters such as the polyhedrin (Polh) promoter, 4IE1 promoter, p5 promoter, p10 promoter p19 promoter, the p40 promoter, metallothionein promoter, 39K promoter, p6.9 promoter, and orf46 promoter.

The term “non-AAV helper function” refers to non-AAV derived viral and/or cellular functions upon which AAV is dependent for its replication. Thus, the term captures proteins and RNAs that are required in AAV replication, including those moieties involved in activation of AAV gene transcription, stage specific AAV mRNA splicing, AAV DNA replication, synthesis of Cap expression products and AAV capsid assembly. Viral-based accessory functions can be derived from any of the known helper viruses such as adenovirus, herpesvirus (other than herpes simplex virus type-1) and vaccinia virus.

The term “non-AAV helper function vector” refers generally to a nucleic acid molecule that includes nucleotide sequences providing accessory functions. An accessory function vector can be transfected into a suitable host cell, wherein the vector is then capable of supporting AAV virion production in the host cell. Expressly excluded from the term are infectious viral particles as they exist in nature, such as adenovirus, herpesvirus or vaccinia virus particles. Thus, accessory function vectors can be in the form of a plasmid, phage, transposon or cosmid. In particular, it has been demonstrated that the full-complement of adenovirus genes are not required for accessory helper functions. For example, adenovirus mutants incapable of DNA replication and late gene synthesis have been shown to be permissive for AAV replication. Ito et al., (1970) J. Gen. Virol. 9:243; Ishibashi et al, (1971) Virology 45:317. Similarly, mutants within the E2B and E3 regions have been shown to support AAV replication, indicating that the E2B and E3 regions are probably not involved in providing accessory functions. Carter et al., (1983) Virology 126:505. However, adenoviruses defective in the E1 region, or having a deleted E4 region, are unable to support AAV replication. Thus, E1A and E4 regions are likely required for AAV replication, either directly or indirectly. Laughlin et al., (1982). J. Virol. 41:868; Janik et al., (1981) Proc. Natl. Acad. Sci. USA 78:1925; Carter et al., (1983) Virology 126:505. Other characterized Ad mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Although studies of the accessory functions provided by adenoviruses having mutations in the E1B coding region have produced conflicting results, Samulski et al., (1988) J. Virol. 62:206-210, recently reported that E1B55k is required for AAV virion production, while E1B19k is not. In addition, International Publication WO 97/17458 and Matshushita et al., (1998) Gene Therapy 5:938-945, describe accessory function vectors encoding various Ad genes. Particularly preferred accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such vectors are described in International Publication No. WO 01/83797.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. (1989) vol. 63, pp.3822-3828; Kajigaya et al., Proc. Nat'l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). In some embodiments, the nucleic acid construct encoding AAV in insect cells is an insect cell-compatible vector. “Expression vector” refers to a vector including a recombinant polynucleotide including expression control sequences operatively linked to a nucleotide sequence to be expressed. An expression vector includes sufficient cis-acting elements for expression; other elements for expression can be supplied by the host cell or in vitro expression system. Expression vectors include all those known in the art, such as cosmids, plasmids (e.g., naked or contained in liposomes), artificial chromosomes, and viruses that incorporate the recombinant polynucleotide. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In a more preferred embodiment, the vector is a baculovirus, i.e. the construct is a baculoviral vector. Baculoviral vectors and methods for their use are described in the above cited references on molecular engineering of insect cells.

The baculovirus shuttle vector or bacmids are used for generating baculoviruses. Bacmids propagate in bacteria such as Escherichia colt as a large plasmid. When transfected into insect cells, the bacmids generate baculovirus.

In some embodiments, the culture medium is an infection or transfection medium (e.g., medium in which the host cell producing the AAV viral particle is infected or transfected with genes (infection or transfection media).

In another embodiment, the culture medium is a producer medium (e.g., medium in which the host cell produces the AAV viral particle). These media include, without limitation, media produced by Life Technologies including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of AAV viral particle.

rAAV particles can also be produced using methods disclosed in various embodiments. In some instances, rAAV particles can be produced by using an insect or mammalian cell that stably expresses some of the necessary components for rAAV particle production. For example, a plasmid (or multiple plasmids) including AAV rep and cap genes, and a selectable marker, such as a neomycin resistance gene, can be integrated into the genome of the cell. In another example, a plasmid (or multiple plasmids) including a selectable marker, such as a neomycin resistance gene, can be integrated into the genome of the cell. The insect, fungal, or mammalian cell can then be co-infected with a helper virus (e.g., adenovirus or baculovirus providing the helper functions) and the viral vector including the 5′ and 3′ AAV ITR (and the nucleotide sequence encoding the heterologous protein, if desired). The advantages of this method are that the cells are selectable and are suitable for large-scale production of the rAAV. As another non-limiting example, adenovirus or baculovirus rather than plasmids can be used to introduce a host regulatory gene, rep gene, and cap gene into packaging cells.

### Host Cells

The host cell can be any invertebrate or vertebrate cell type which allows for production of the AAV viral particle and which can be maintained in culture.

In one embodiment, the host cell is an insect cell or a mammalian cell.

In another embodiment, the host cell is an insect cell.

In another embodiment, the mammalian cell is a human cell.

In another embodiment, the mammalian cell is HEK293, HeLa, CHO, NSO, SP2/0, PER.C6, Vera, RD, BHK, HT 1080, A549, Cos-7, ARPE-19 or MRC-5 cells.

In some embodiments, the insect cell is from Spodoptera frugiperda, such as Sf9, Sf21, Sf900+, drosophila cell lines, mosquito cell lines, for example, Aedes albopictus derived cell lines, domestic silkworm cell lines, for example, Bombyxmori cell lines, Trichoplusia ni cell lines such as High Five cells or Lepidoptera cell lines such as Ascalapha odorata cell lines. Preferred insect cells are cells from the insect species which are susceptible to baculovirus infection, including High Five, Sf9, Se301, SeIZD2109, SeUCR1, Sf900+, Sf21, BTI-TN-5B 1-4, MG-1, Tn368, HzAm1, BM-N, Ha2302, Hz2E5 and Ao38.

### AAV Serotypes

There are at least thirteen serotypes of AAV that have been characterized, as shown in Table 2. The instant invention encompasses but is not limited to these specific AAV serotypes.

TABLE 2
AAV Serotypes.

| | NCBI Reference Sequence No./ |
|---|---|
| | Genbank Accession No. |
| AAV Serotype | (each herein incorporated by reference) |
| AAV-1 | NC_002077.1 |
| AAV-2 | NC_001401.2 |
| AAV-3 | NC_001729.1 |
| AAV-3B | AF028705.1 |
| AAV-4 | NC_001829.1 |
| AAV-5 | NC_006152.1 |
| AAV-6 | AF028704-1 |
| AAV-7 | NC_006260.1 |
| AAV-8 | NC_006261.1 |
| AAV-9 | AX7S3250.1 |
| AAV-10 | AY631965.1 |
| AAV-11 | AY631966.1 |
| AAV-12 | DQ813647.1 |
| AAV-13 | EU28SS62.1 |

General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV-6. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control.

AAV “rep” and “cap” genes are genes encoding replication and encapsidation proteins, respectively. AAV rep and cap genes have been found in all AAV serotypes examined and are described herein, and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap gene in accordance with the present invention encodes a Cap protein which is capable of packaging AAV vectors in the presence of rep and adeno helper function and is capable of binding target cellular receptors. In some embodiments, the AAV cap gene encodes a capsid protein having an amino acid sequence derived from a particular AAV serotype, for example the serotypes shown in Table 2; or derived from alternative capsid variant sequences of AAV found in mammals e.g., humans, baboons, pigs, marmosets, chimpanzees, or macaques (e.g., rhesus (Macaca mulatta), cynomolgus (“long-tailed”) (M. fascicularis), or pigtailed (M. nemestrina)).

The AAV sequences employed for the production of AAV can be derived from the genome of any AAV serotype. The AAV serotypes may have genomic sequences of significant homology at the amino acid and the nucleic acid levels, provide a similar set of genetic functions, produce virions which are essentially physically and functionally equivalent, and replicate and assemble by practically identical mechanisms. See, for example, GenBank Accession number U89790; GenBank Accession number J01901; GenBank Accession number AF043303; GenBank Accession number AF085716; Chlorini et al., J. Vir. 71: 6823-33 (1997); Srivastava et al., J. Vir. 45:555-64 (1983); Chlorini et al., J. Vir. 73:1309-1319 (1999); Rutledge et al., J. Vir. 72:309-319 (1998); and Wu et al., J. Vir. 74: 8635-47 (2000), which are herein incorporated by reference for the genomic sequences of AAV serotypes and/or discussions of the genomic similarities.

The genomic organization of many of the known AAV serotypes can be very similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In various embodiments, a vector providing AAV helper functions includes a nucleotide sequence(s) that encode capsid proteins, Rep proteins, or AAP proteins. The cap genes, rep gene, and/or AAP gene from any AAV serotype (including, but not limited to, AAV1 (NCBI Reference Sequence No./Genbank Accession No. NC_002077.1), AAV2 (NCBI Reference Sequence No./Genbank Accession No. NC_001401.2), AAV3 (NCBI Reference Sequence No./Genbank Accession No. NC_001729.1), AAV3B (NCBI Reference Sequence No./Genbank Accession No. AF028705.1), AAV4 (NCBI Reference Sequence No./Genbank Accession No. NC_001829.1), AAV5 (NCBI Reference Sequence No./Genbank Accession No. NC_006152.1), AAV6 (NCBI Reference Sequence No./Genbank Accession No. AF028704.1), AAV7 (NCBI Reference Sequence No./Genbank Accession No. NC_006260.1), AAV8 (NCBI Reference Sequence No./Genbank Accession No. NC_006261.1), AAV9 (NCBI Reference Sequence No./Genbank Accession No. AX753250.1), AAV10 (NCBI Reference Sequence No./Genbank Accession No. AY631965.1), AAV11 (NCBI Reference Sequence No./Genbank Accession No. AY631966.1), AAV12 (NCBI Reference Sequence No./Genbank Accession No. DQ813647.1), AAV13 (NCBI Reference Sequence No./Genbank Accession No. EU285562.1), is AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAV_po.6, AAV_po., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, AAVanc80L65, or any variants thereof) can be used herein to produce the recombinant AAV Exemplary capsids are also provided in International Application No. WO 2018/022608 and WO 2019/222136, which are incorporated herein in its entirety. Each NCBI Reference Sequence Number or Genbank Accession Numbers provided above is also incorporated by reference herein. In some embodiments, the AAV cap genes encode a capsid from serotype 1, serotype 2, serotype 3, serotype 3B, serotype 4, serotype 5, serotype 6, serotype 7, serotype 8, serotype 9, serotype 10, serotype 11, serotype 12, serotype 13, or a variant thereof.

In addition to the capsid, Rep, and AAP genes, embodiments include exogenous polynucleotides that express helper proteins. Without limitation, helper gene products that can be expressed in the host cell in various combinations include Spodoptera frugiperda FKBP46, human FKBP52, Adenovirus E1A, E1B, E2A, E4 and VA, Herpes simplex virus UL29, UL30, UL42, U15, UL8, UL52, and UL9. In an embodiment, the cell expresses at least one immunophilin analogue (i.e., an immunophilin or similar protein) and at least one helper virus gene product.

In some embodiments, the three AAV capsid proteins, namely VP1, VP2, and VP3, are produced in an overlapping fashion from the cap open reading frame (ORF) using alternative mRNA splicing of the transcript and alternative translational start codon usage. For example, VP1 can be translated from an ATG start codon (amino acid M1) on the mRNA, while VP2 and VP3 can arise from a shorter mRNA, for example, using a different start codon for VP2 production and readthrough translation to the next available start codon for the production of VP3.

The Cap proteins can be VP1 and VP3, or VP1, VP2, and VP3. The VP1, VP2 or VP3 genes can express capsid proteins of AAV serotypes AAV1, AAV2, AAV4, AAV5, AAV6, AAV7, AAV8, AAV9, AAV10, AAV11, AAV12, AAV13, AAV-rh.10 (AAVrh10), AAV-DJ (AAVDJ), AAV-DJ8 (AAVDJ8), AAV-1, AAV-2, AAV-2G9, AAV-3, AAV3a, AAV3b, AAV3-3, AAV4, AAV4-4, AAV-5, AAV-6, AAV6.1, AAV6.2, AAV6.1.2, AAV-7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV-10, AAV-11, AAV-12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1. 14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV10, or Japanese AAV10 serotypes, AAVpo.6, AAVpo., AAV_po.5, AAV_LK03, AAV_ra.1, AAV_bat_YNM, AAV_bat_Brazil, AAV_mo.1, AAV_avian_DA-1, or AAV_mouse_NY1, Bba21, Bba26, Bba27, Bba29, Bba30, Bba31, Bba32, Bba33, Bba34, Bba35, Bba36, Bba37, Bba38, Bba41, Bba42, Bba43, Bba44, Bce14, Bce15, Bce16, Bce17, Bce18, Bce20, Bce35, Bce36, Bce39, Bce40, Bce41, Bce42, Bce43, Bce44, Bce45, Bce46, Bey20, Bey22, Bey23, Bma42, Bma43, Bpo1, Bpo2, Bpo3, Bpo4, Bpo6, Bpo8, Bpo13, Bpo18, Bpo20, Bpo23, Bpo24, Bpo27, Bpo28, Bpo29, Bpo33, Bpo35, Bpo36, Bpo37, Brh26, Brh27, Brh28, Brh29, Brh30, Brh31, Brh32, Brh33, Bfm17, Bfm18, Bfm20, Bfm21, Bfm24, Bfm25, Bfm27, Bfm32, Bfm33, Bfm34, Bfm35, AAV-rh10, AAV-rh39, AAV-rh43, or AAVanc80L65. In another embodiment, the VP1, VP2, or VP3 genes express a capsid of a mixed serotype wherein at the VP1, VP2, and VP3 genes do not all come from the same serotype. Exemplary capsids are provided in International Application No. WO 2018/022608, incorporated herein in its entirety.

### Baculovirus virions

In some embodiments, a baculoviral system is employed.

Baculoviruses are enveloped DNA viruses of arthropods, two members of which are well known for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedro virus (AcMNPV) or Bombyx mori (Bm)NPV).

Baculoviruses are commonly used for the infection of insect cells for the expression of recombinant proteins. In particular, expression of heterologous genes in insects can be accomplished as described in for instance U.S. Pat. No. 4,745,051; Friesen et al (1986); EP 127,839; and EP 155,476. Numerous baculovirus strains and variants and corresponding permissive insect host cells that can be used for protein production are known in the art.

For example, the commercially available Bac-to-Bac® system (Thermo Fisher Scientific, Rockford, IL) (Catalog No. 10359016) includes expression vectors for recombinant protein expression. The pFastBac™ 1 vector (Thermo Fisher Scientific, Rockford, IL) has the strong polyhedrin promoter for high-level protein expression and a large multiple cloning site for simplified cloning. The pFastBac™ Dual (Thermo Fisher Scientific, Rockford, IL) is a single vector featuring two strong promoters, the polyhedrin promoter and the p10 promoter in a single vector for simultaneous expression of two proteins in insect cells.

Briefly, by way of illustration, a baculoviral system such as Bac-to-Bac® relies on the generation of recombinant baculovirus by site-specific transposition in E. coli rather than homologous recombination in insect cells. For example, a gene of interest can be cloned into a pFastBac™ vector and transformed into DH10Bac™ competent E. coli (Thermo Fisher Scientific, Rockford, IL). DH10Bac™ contains a parent bacmid with a lacZ-mini-attTn7 fusion. Transposition occurs between the elements of the pFastBac™ vector and the parent bacmid in the presence of the transposition proteins provided by a helper plasmid. When the transposition is successful, the expression cassette disrupts the lacZ gene and the new expression bacmid can be visualized as white bacterial colonies. The new expression bacmid can be isolated and used to transfect, for example, Sf9 or Sf21 cells using a transfection reagent of any embodiment. After an appropriate amount of time in culture, recombinant baculovirus can be isolated. The recombinant baculovirus can be used to infect the cell to produce AAV viral particles and/or to express gene(s) of interest.

### Purification of AAV Viral Particles

In other embodiments, AAV viral particles can be purified from the host cell using a variety of conventional purification methods, such as column chromatography, CsCl gradients, and the like. For example, a plurality of column purification steps can be used, such as purification over an anion exchange column, an affinity column, and/or a cation exchange column. See, for example, International Publication No. WO 02/12455. Further, if infection is employed to express the accessory functions, residual helper virus can be inactivated, using known methods. For example, adenovirus can be inactivated by heating to temperatures of approximately 60° C. for, for example, 20 minutes or more. This treatment effectively inactivates only the helper virus since AAV is extremely heat stable while the helper adenovirus is heat labile.

In one embodiment, the AAV viral particle stock is then treated to remove empty capsids, for example, using column chromatography techniques.

In another embodiment, AAV viral particle preparations are obtained by lysing transfected cells to obtain a crude cell lysate. The crude cell lysate can then be clarified to remove cell debris by techniques well known in the art, such as filtering, centrifuging, and the like, to render a clarified cell lysate. The crude cell lysate or clarified cell lysate, which may contain both AAV viral particles and AAV empty capsids, can then be applied to a first cation exchange matrix under non-separating conditions, wherein the first cation exchange column functions to further separate the AAV viral particles and the AAV empty capsids from cellular and other components present in the cell lysate preparation. Methods for performing the initial purification of the cell lysate are known. One representative method is described in U.S. Pat. No. 6,593,123, herein incorporated by reference in its entirety.

### Analytical Techniques

Generally, vg and capsid (cp) titers may be evaluated in any way that is suitable for measuring the respective vg and capsids. For example, quantitative polymerase chain reaction (qPCR) may be used to measure vg titers and enzyme-linked immunosorbent assay (ELISA) may be used to measure Cp titer. Alternatively, SEC (size-exclusion chromatography)-HPLC may be used to measure the vg and cp titers. In addition, RP (reverse phase)-HPLC or capillary electrophoresis assays may be used to evaluate the potential impact of process parameters on VP ratios.

qPCR may be used for vg quantification by quantitative polymerase chain reaction (qPCR) using a standard qPCR system, such as an Applied Biosystems 7500 Fast Real-Time PCR system. Alternatively, digital droplet PCR (ddPCR) may be used for Vg quantification. Primers and probes may be designed to target the DNA of the AAV, allowing its quantification as it accumulates during PCR. Examples of ddPCR are described in Pasi, K. John, et al. “Multiyear Follow-Up of AAV5-hFVIII-SQ Gene Therapy for Hemophilia A.” New England Journal of Medicine 382.1 (2020): 29-40; Regan, John F., et al. “A Rapid Molecular Approach for Chromosomal Phasing.” PloS one 10.3 (2015): 00118270; and Furuta-Hanawa, Birei, Teruhide Yamaguchi, and Eriko Uchida. “Two-Dimensional Droplet Digital PCR as a Tool for Titration and Integrity Evaluation of Recombinant Adeno-Associated Viral Vectors” Human gene therapy methods 30.4 (2019): 127-136. Other systems for vg quantification include SEC, SEC-HPLC, and size exchange chromatography multi-angle light scattering, all of which are described in WO 2021/062164, which is incorporated in its entirety by reference.

The capsid ELISA (cp-ELISA) assay measures intact capsids using, e.g., the AAV5 or AAV5 Capsid ELISA method and may utilize a commercially-available kit (for example, Progen PRAAV5). This kit ELISA employs a monoclonal antibody specific for a conformational epitope on assembled AAV5 or other capsids. Capsids can be captured on a plate-bound monoclonal antibody, followed by subsequent binding of a detection antibody. The assay signal may be generated by addition of conjugated streptavidin peroxidase followed by addition of colorimetric TMB substrate solution, and sulfuric acid to end the reaction. The titers of test samples are interpolated from a four-parameter calibration curve of the target capsid standard. Another system for quantifying capsid titers is SEC-MALS, which are described in WO 2021/062164.

The titer of the rBV can be determined using a foci/viral plaque assay. This assay first includes the step of infecting cells with serial dilutions of a solution containing rBV. After infection occurs for a predetermined time, the rBV is removed from the cultures and the cells are incubated for a pre-determined time. After the pre-determined time has elapsed, a plaquing media (e.g., containing agarose) is added to the cultures and allowed to harden. The cells are allowed to further incubate for a pre-determined time and the number of plaques are counted after the pre-determined time. The titer is calculated using the following formula: Titer (plaque forming units/mL)=number of plaques×dilution factor×(1/(mL of inoculum/well)

### Cationic Peptides and Histidine Rich Peptides

The methods include the use of cationic peptides as transfection reagents, where the peptides have a positive charge at a pH ranging from 6 to 8 (e.g., 7.4). A suitable transfection reagent should meet several criteria to be a successful candidate for large scale viral vector production: 1) preferably bind the foreign nucleic acids provided in the forms of plasmids, bacmids or any other forms; 2) complexes of foreign DNA and the transfection reagent is preferably stable for more than 10 minutes; 3) complexes of foreign DNA:transfection reagent bind to the cell surface and be taken up by the cells; 4) transfection reagent preferably provides a way for endosomal release of the foreign DNA or foreign DNA: transfection reagent complexes; 5) foreign DNA or foreign DNA: transfection reagent complexes is preferably able to reach the nucleus of the cell; 6) foreign DNA or foreign DNA:transfection reagent complexes is preferably available for transcription of the proteins and replication of the genes; 7) the transfection reagent and foreign DNA: transfection reagent complexes is preferably well tolerated by the cells; 8) foreign DNA or foreign DNA:transfection reagent complexes is preferably compatible with the production media of choice; and 9) the transfection reagent is preferably able to transfect the production cells in the scalable manner.

Examples of cationic peptides include histidine rich peptides (HRPs). HRPs have been used for the delivery of different types of cargo into the cells (Moulay G. et al., Histidine-rich designer peptides of the LAH4 family promote cell delivery of a multitude of cargo. Journal of Peptide Science, 2017, 23(4):320-328).

As used herein, the terms “polypeptide,” “peptide,” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

However, HRPs have not been used as a transfection reagent for viral vector production in mammalian or insect cells before.

HRPs are about 20 amino acids long and contain hydrophobic amino acid residues which facilitate their binding to the cellular membranes, hydrophilic residues which facilitate their solubility in water and several histidine residues (four histidine residues for the well characterized member of the family, LAH4). Histidines at neutral pH are positively charged and can bind to the negatively charged cargo. This makes the overall charge of the cargo:peptide complex positive. A positive charge ratio of (+/−) 3.6 was seen for LAH4 and a given plasmid [4]. This positive charge of cargo:peptide complex facilitates the binding to the cellular plasma membrane which is generally considered negatively charged. It was also reported that receptor mediated endocytosis can occur through glycoproteins on the surface of the cells (Kichler A. et al., Cationic amphipathic histidine-rich peptides for gene delivery, Biochimica et biophysica acta 2006, 1758(3):301-307). The endocytosed DNA:peptide complex moves along the endocytotic pathways from early endosome to late endosome. Along the pathway the pH gets more acidic and histidines get more protonated changing their net charge from +5 to +9 (for LAH4 peptide). That causes approximately half of the peptide to dissociate from DNA:peptide complexes. Released free peptides destabilize the endosomal membranes and allow DNA containing complexes to escape into the cytosol.

HRP are histidine-peptides that are approximately 10-50 amino acids in length. Suitable HRP have a nominal charge at pH 7.4 of 5, 6, 7, 8, 9 or 10 and a nominal charge at pH 5 of 6, 7, 8, 9 or 10. HRP further have a hydrophobic moment at pH 7 of 0.02-0.4, e.g., the hydrophobic moment at pH 7 may be 0.03, 0.04, 0.05. 0.06, 0.07, 0.08, 0.09, 0.10, 0.11, 0.12, 0.13. 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.,49, 0.50; and a polar angle of 20-160, e.g., approximately, 10, 20, 30, 40, 50, 60, 70, 80, 90, 100, 110, 120, 130, 140, 150, 160. Preferred HRP have a core made of alanines and leucines interspersed with four histidines.

In various examples, at least a portion of the HRP forms a-helical conformation when in the presence of cell membranes and when represented according to Schiffer-Edmundson's wheel representation (Schiffer M, Edmundson A B. Biophys J. 1967 March;7(2):121-35). The α-helix can be an amphipathic helix harboring a cluster of hydrophobic amino acid residues on one side of the helix and two to four histidine residues on the other side of the helix, defining a hydrophilic angle comprised between 60° and 180° in Schiffer-Edmundson's wheel representation. The α-helix can also be an apolar helix harboring a cluster of hydrophobic amino acid residues on one side of the helix and consecutive alanine residues on the other side of the helix, said consecutive alanine residues defining an angle of 60 to 180° in Schiffer-Edmundson's wheel representation. In other examples, the portions N-terminus and C-terminus of the HRP include one or more amino acid residues that are positively charged at pH 7.4. For example, the LAH4 peptide (SEQ ID NO: 1) has a lysine or arginine residues at the N-terminus and C-terminus. See International publication WO 2013/001041, which is incorporated by reference in its entirety by reference.

For the purpose of transfecting DNA, the characteristics of exemplary HRP include peptides that are relatively short, present cationic residues which allow electrostatic interactions with DNA but with a limited positive charge density, be soluble in aqueous solutions, and be able to interact with and destabilize membranes. See International publication WO 2002/096928; Kichler, Antoine, A. James Mason, and Burkhard Bechinger. Biochimica et Biophysica Acta (BBA)-Biomembranes 1758.3 (2006): 301-307; Kichler, Antoine, et al. Journal of Molecular Medicine 85.2 (2007): 191-201. All of these references are incorporated in their entirety by reference.

In various embodiments, the cationic peptide or HRP includes a covalent modification. Examples of covalent modifications include acylation, acetylation, linkage (e.g., to the N-terminus) to a non-peptidic macro molecular carrier group; amidation, linkage (e.g., to the C-terminus) to a non-peptidic macro molecular carrier group; glycosylation (e.g., of amino acid side chains); linkage to an adaptor protein that can promote uptake of the peptide into cells or linkage to a hydrophobic group such as a lipid, a fatty acid, a dansyl, a carbobenzoxyl or a t-butyloxycarbonyl group; oxidation, sulphatization, esterification, lactone formation and/or phosphorylation. In other embodiments, the cationic peptide includes a marker that allows for quantification of the cationic peptide. For example, the cationic peptide can include tyrosine (Tyr or Y) or tryptophan (Trp or W).

It is noted that the cationic peptides as described in various embodiments are not limited to the HRPs and also include other cationic peptides that are also described in various embodiments. For example, various cationic peptides or HRPs are disclosed in table 3.

TABLE 3
Cationic Peptides and Histidine Rich Peptides

| | Alter- | SEQ | |
|---|---|---|---|
| | native | ID | |
| Amino Acid Sequence | Name | NO: | Comment |
| KKALLALALHHLAHLALHLALALKKA | LAH4 | 1 | |
| KKALLAHALHLLALLALHLAHALKKA | LAH4-L1 | 2 | |
| KKALLALALHHLAHLAHHLALALKKA | LAH5 | 3 | |
| KKALLAHFFHLLALLALHFFHALKKA | LAH4-L1- | 4 | |
| | F4 | | |
| KKALLAHFLHLLALLALHLFHALKKA | LAH4-L1- | 5 | |
| | F2D | | |
| KKALLALALHHALHLALHLALALKKA | LAH4 | 6 | |
| | INV-AL | | |
| KKALAHALHLLALALLHLAHALAKK | LAH4-L1-β | 7 | |
| KKLAKALAKALAKALKLALALAKK | LAK4 | 8 | |
| KKLALALALALHALALALALKKA | LAH1 | 9 | |
| KKLAHLALALALGLALAHLAKKA | LAH2 | 10 | |
| KKALALGLHLAHLALHLALALKKA | LAH3 | 11 | |
| KKALLALALHHLAHPALHLALALKKA | LAH4-P15 | 12 | |
| KKALLALALHHLAHLALHLALALKKA | D-LAH4 | 13 | D-enantiomer |
| KKLALAHALHLALLLALHLAHALKKA | LAH4-L1- | 14 | |
| | Opt | | |
| KKALLALAXHHLAHLALHXALALKKA | LAH4X2 | 15 | X is α-amino |
| | | | butyric acid |
| KKALLALAXHHLAHLAHHXALALKKA | LAH5X2 | 16 | X is α-amino |
| | | | butyric acid |
| KKALLALAXKKWAKLWKKXALALKKA | LAK5X2W | 17 | X is α-amino |
| | 2 | | butyric acid |
| KKALLALALHHLALLAHHLALALKKA | | 18 | |
| KKALLALALHHLALLAHLLALHLKKA | | 19 | |
| KKKGLFHAIHGFIHNGWHGMIHGWYGKKK | | 20 | |
| RRRRRRRRGLFHAIHGFIHNGWHGMIHGWYG | | 21 | |
| CKKKGLFHAIHGFIHNGWHGMIHGWYGKKKC | | 22 | |
| KKKGLFHALLHLLHSLWHLLLHAKKK | | 23 | |
| KKALLAHALHLLALLALHLAHALA | | 24 | |
| RRALLAHALHLLALLALHLAHALRRA | | 25 | |
| KKALLAHALAHLALLALHLALHLKKA | | 26 | |
| KKALLALALHHLALLALHLAHALKKA | | 27 | |
| KKALLHAALAHLLPLAHHLLALLKKA | | 28 | |
| KKALLAKALKLLALLALKLAKALKKA | | 29 | |
| HHALLAHALHLLALLALHLAHALHHA | | 30 | |
| ALLAHALHLLALLALHLAHALA | | 31 | |
| ALLAHALHLLALLALHLAHALKKA | | 32 | |
| ALLHAALAHLLALAHHLLALLKKA | | 33 | |
| KKALLAAALAALLALAHHLLALLKKA | | 34 | |
| GIHKQKEKSRLQGGVLVNEILNHMKRATQIPSYKK | | 35 | |
| LIMY | | | |
| KKKKALLHLHLLALHLHLLALLALKKK | | 36 | |
| KWKLFKKIGAVLKVLTTG | | 37 | |
| CLLKKLLKKLLKKC | | 38 | |
| WEAKLAKALAKALAKHLAKALAKALKACEA | | 39 | |
| YGRKKRRQRRRC | | 40 | |
| RQIKIWFQNRRMKWKKC | | 41 | |
| YARAAARQARA | | 42 | |
| HHHHHHYARAAARQARA | | 43 | |
| YGRKKRRQRRRCKWKLFKKIGAVLKVLTTG | | 44 | |
| YGRKKRRQRRRCWEAKLAKALAKALAKHLAKAL | | 45 | |
| AKALKACEA | | | |
| YARAAARQARAWEAKLAKALAKALAKHLAKALA | | 46 | |
| KALKACEA | | | |
| RRRRRRRRRWEAKLAKALAKALAKHLAKALAKAL | | 47 | |
| KACEA | | | |
| KETWWETWWTEWSQPKKKRKVWEAKLAKALAK | | 48 | |
| ALAKHLAKALAKALKACEA | | | |
| LCLRPVGWEAKLAKALAKALAKHLAKALAKALKA | | 49 | |
| CEA | | | |
| RRLSYSRRRFWEAKLAKALAKALAKHLAKALAKA | | 50 | |
| LKACEA | | | |
| KWKLFKKIGAVLKVLTTGYGRKKRRQRRRC | | 51 | |
| KWKLFKKIGAVLKVLTTGRQIKIWFQNRRMKWKK | | 52 | |
| C | | | |
| KWKLFKKIGAVLKVLTTGYGRKKRRQRRRC | | 53 | Includes dimers |
| | | | linked via |
| | | | disulfide bridge |
| | | | between |
| | | | cysteine(s) |
| KWKLFKKIGAVLKVLTTGRQIKIWFQNRRMKWKK | | 54 | Includes dimers |
| C | | | linked via |
| | | | disulfide bridge |
| | | | between |
| | | | cysteine(s) |
| KFTIVFPHNQKGNWKNVPSNYHYCPYARAAARQA | | 55 | |
| RA | | | |
| LIRLWSHLIHIWFQNRRLKWKKKYARAAARQARA | | 56 | |
| GLFEALLELLESLWELLLEAYARAAARQARA | | 57 | |
| KWKLFKKIGAVLKVLTTGYARAAARQARA | | 58 | |
| CCCCCCKWKLFKKIGAVLKVLTTGYARAAARQAR | | 59 | |
| A | | | |
| KWKLFKKIGAVLKVLTTGGGSYARAAARQARA | | 60 | |
| KWKLFKKIGAVLKVLTTGGGSGGGSYARAAARQA | | 61 | |
| RA | | | |
| KWKLFKKIGAVLKVLTTGGGSGGGSGGGSGYARA | | 62 | |
| AARQARA | | | |
| MHHHHHHKWKLFKKIGAVLKV | | 63 | |
| LTTGYGRKKRRQRRRC | | | |
| HHHHHHKWKLFKKIGAVLKVLTTGYGRKKRRQRR | | 64 | |
| R | | | |
| HHHHHHKWKLFKKIGAVLKVLTTGYARAAARQAR | | 65 | |
| A | | | |
| HHHHHHKWKLFKKIGAVLKVLT | | 66 | |
| TGYARAAARQARACCCCCC | | | |
| HHHHHHKWKLFKKIGAVLKVLTTGRRRRRRRRR | | 67 | |
| HHHHHHKWKLFKKIGAVLKVLT | | 68 | |
| TGGWTLNSAGYLLKINLKALAALAKKIL | | | |
| HHHHHHKKALLALALHHLAHLALHLALALKKAYA | | 69 | |
| RAAARQARA | | | |
| HHHHHHCLLKKLLKKLLKKCYARAAARQARA | | 70 | |
| HHHKWKLFKKIGAVLKVLTTGYARAAARQARA | | 71 | |
| HHHHHHHHHHHHKWKLFKKIGAVLKVLTTGYAR | | 72 | |
| AAARQARA | | | |
| HHHAHHHKWKLFKKIGAVLKVLTTGYARAAARQ | | 73 | |
| ARA | | | |
| HAHHAHHAHKWKLFKKIGAVLKVLTTGYARAAA | | 74 | |
| RQARA | | | |
| KWKLFKKIGAVLKVLTTGHHHH | | 75 | |
| HHYARAAARQARA | | | |
| HHHHHHKWKLFKKIGAVLKVLT | | 76 | |
| TGYARAAARQARAHHHHHH | | | |
| HHHHHHYARAAARQARAGGGGSKKALLALALHH | | 77 | |
| LAHLALHLALALKKA | | | |
| YARAAARQARAGGGGSKKALLALALHHLAHLALH | | 78 | |
| LALALKKA | | | |
| HHHHHHRQIKIWFQNRRMKWKKGGGGGGSKKAL | | 79 | |
| LALALHHLAHLALHLALALKKA | | | |
| RQIKIWFQNRRMKWKKGGGGGGSKKALLALALHH | | 80 | |
| LAHLALHLALALKKA | | | |
| HHHHHHYARAAARQARAGGGGSKKALLAHFFHLL | | 81 | |
| ALLALHFFHALKKA | | | |
| YARAAARQARAGGGGSKKALLAHFFHLLALLALH | | 82 | |
| FFHALKKA | | | |
| HHHHHHRQIKIWFQNRRMKWKKGGGGGGSKKAL | | 83 | |
| LAHFFHLLALLALHFFHALKKA | | | |
| RQIKIWFQNRRMKWKKGGGGGGSKKALLAHFFHL | | 84 | |
| LALLALHFFHALKKA | | | |
| KKWALLALALHHLAHLALHLALALKKA | | 85 | |
| KKWALLAHFFHLLALLALHFFHALKKA | | 86 | |
| PKKKRKVGGGGSKKALLALALHHLAHLALHLALA | | 87 | |
| LKKA | | | |
| PKKKRKVGGGGSKKALLAHFFHLLALLALHFFHAL | | 88 | |
| KKA | | | |
| KRPAATKKAGQAKKKKGGGGSKKALLALALHHLA | | 89 | |
| HLALHLALALKKA | | | |
| KRPAATKKAGQAKKKKGGGGSKKALLAHFFHLLA | | 90 | |
| LLALHFFHALKKA | | | |
| PARKRLNGGGGSKKWALLALALHHLAHLALHLAL | | 91 | |
| ALKKA | | | |
| PARKRLNGGGGSKKALLAHFFHLLALLALHFFHAL | | 92 | |
| KKA | | | |
| HHHHHHYARAAARQARAGGGGSKKALLALALHH | His-PTD4- | 93 | |
| LAHLALHLALALKKA | LAH4 | | |
| YARAAARQARAGGGGSKKALLALALHHL | PTD4- | 94 | |
| | LAH4 | | |
| HHHHHHRQIKIWFQNRRMKWKKGGGGGGSKKAL | His- | 95 | |
| LALALHHLAHLALHLALALKKA | Penetratin- | | |
| | LAH4 | | |
| RQIKIWFQNRRMKWKKGGGGGGSKKALLALALHH | Penetratin- | 96 | |
| LAHLALHLALALKKA | LAH4 | | |
| HHHHHHYARAAARQARAGGGGSKKALLAHFFHLL | His-PTD4- | 97 | |
| ALLALHFFHALKKA | LAH4-L1- | | |
| | F4 | | |
| YARAAARQARAGGGGSKKALLAHFFHLLALLALH | PTD4- | 98 | |
| FFHALKKA | LAH4-L1- | | |
| | F4 | | |
| HHHHHHRQIKIWFQNRRMKWKKGGGGGGSKKAL | His- | 99 | |
| LAHFFHLLALLALHFFHALKKA | Penetratin- | | |
| | LAH4-L1- | | |
| | F4 | | |
| RQIKIWFQNRRMKWKKGGGGGGSKKALLAHFFHL | Penetratin- | 100 | |
| LALLALHFFHALKKA | LAH4-L1- | | |
| | F4 | | |
| KKWALLALALHHLAHLALHLALALKKA | LAH4(W) | 101 | |
| KKWALLAHFFHLLALLALHFFHALKKA | LAH4-L1- | 102 | |
| | F4(W) | | |
| PKKKRKVGGGGSKKALLALALHHLAHLALHLALA | SV40 T | 103 | |
| LKKA | NLS- | | |
| | spacer- | | |
| | LAH4 | | |
| PKKKRKVGGGGSKKALLAHFFHLLALLALHFFHAL | SV40 T | 104 | |
| KKA | NLS- | | |
| | spacer- | | |
| | LAH4-L1- | | |
| | F4 | | |
| KRPAATKKAGQAKKKKGGGGSKKALLALALHHLA | nucleo- | 105 | |
| HLALHLALALKKA | plasmin | | |
| | NLS-spacer- | | |
| | LAH4 | | |
| KRPAATKKAGQAKKKKGGGGSKKALLAHFFHLLA | nucleo- | 106 | |
| LLALHFFHALKKA | plasmin | | |
| | NLS-spacer- | | |
| | LAH4-L1- | | |
| | F4 | | |
| PARKRLNGGGGSKKWALLALALHHLAHLALHLAL | AAV2 | 107 | |
| ALKKA | VP1-2 | | |
| | BR3- | | |
| | spacer- | | |
| | LAH4 | | |
| PARKRLNGGGGSKKALLAHFFHLLALLALHFFHAL | AAV2 | 108 | |
| KKA | VP1-2 | | |
| | BR3- | | |
| | spacer- | | |
| | LAH4-L1- | | |
| | F4 | | |
| KKALLAHALAHLALLALHLALHLKKA | LAH4-L0 | 109 | |
| KKALLALALHHLALLALHLAHALKKA | LAH4-L2 | 110 | |
| KKALLALALHHLALLAHHLALALKKA | LAH4-L3 | 111 | |
| KKALLHLALLHAALLAHHLALALKKA | LAH4-L4 | 112 | |
| KKALLHLALLHAALLAHLAALHLKKA | LAH4-L5 | 113 | |
| KKALLHLALLLAALHAHLAALHLKKA | LAH4-L6 | 114 | |
| KKALLAHALHLLAALALHLAHLLKKA | LAH4-A1 | 115 | |
| KKALLLAALHHLAALALHLAHLLKKA | LAH4-A2 | 116 | |
| KKALLLAALHHLLALAHHLAALLKKA | LAH4-A3 | 117 | |
| KKALLHAALAHLLALAHHLLALLKKA | LAH4-A4 | 118 | |
| KKALLHALLAHLAALLHALLAHLKKA | LAH4-A5 | 119 | |
| KKALLHALLAALLAHLHALLAHLKKA | LAH4-A6 | 120 | |
| HHHHHHRQIKIWFQNRRMKWKKGGGGSKKALLA | His- | 121 | |
| LALHHLAHLALHLALALKKA | Penetratin- | | |
| | LAH4-2 | | |
| RQIKIWFQNRRMKWKKGGGGSKKALLALALHHLA | Penetratin- | 122 | |
| HLALHLALALKKA | LAH4-2 | | |
| HHHHHHRQIKIWFQNRRMKWKKGGGGSKKALLA | His- | 123 | |
| HFFHLLALLALHFFHALKKA | Penetratin- | | |
| | LAH4-L1- | | |
| | F4-2 | | |
| RQIKIWFQNRRMKWKKGGGGSKKALLAHFFHLLA | Penetratin- | 124 | |
| LLALHFFHALKKA | LAH4-L1- | | |
| | F4-2 | | |

Other examples of cationic peptides and HRPs that can be used as transfection reagents are disclosed in International publication WO 2017/175072, U.S. Pat. No. 8,652,483, and Mello, Lucas R., et al. “Self-assembly and intracellular delivery of DNA by a truncated fragment derived from the Trojan peptide Penetratin.” Soft matter 16.20 (2020): 47464755. All of these references are incorporated in their entirety by reference.

The efficiency of the transfection process can be expressed in many ways, for example by the % of the cells producing a protein from the gene which was delivered with foreign DNA, number of copies of foreign DNA detected in the cells after transfection, enzymatic activity of the protein produced from the delivered gene and other ways. For example, transfection efficiency may be determined as % Green Fluorescence Protein positive (GFP⁺) cells at a given time point after transfection of plasmid carrying the gene for GFP.

HRPs may be used to improve transfection with the product of AAV in insect cells, such as sf9 cells. In this regard, after bacmids containing the gene of interest are produced the plasmids from the bacmids may be used along with the HRP transfection agent to create baculovirus infected insect cells (BIICs). When expansion is needed the previously cryopreserved BIIC can be expanded with, insect cells, and AAV capsids containing therapeutic gene produced. Thus, with the case of insect cells the transfection agent may be used at the upstream stage of creating BIICs.

HRPs may also be used to improve the transfection with mammalian cells. In this case, the HRP is along with a plasmid to transfect a mammalian cell, such as HEK293 cells.

HRPs have the advantages of having a long stability time and being suitable at low transfection volumes. In this regard HRPs may be used at a 1% transfection volume and are stable for at least 60 minutes. Unlike other many other transfection reagents such as PEI and FECTOVIR®, HRP of a GMP grade are also commercially available. In addition, unlike PEI and FECTOVIR®, which is presumed to remain in the cells, HRPs are broken down in the cells and have low cytotoxicity. HRPs may also be used for transfection at high cell density culture, e.g., 2-8×10⁶ cells/ml, with a high transfection efficiency of at least 70% and up to 90% or more. Thus, the transfection efficiency is about 70%, 75%, 80%, 85%, or 90%. With the transfection method of the invention using the HRP transfection reagent vector/transfection reagent complexes that are stable for more than 10 minutes, or more than 15 minutes, more than 20 minutes, more than 25 min. One advantage provided by the HRP transfection reagent is the low volume, which may be used. In this regard, the vectors for rAAV production and transfection reagent are mixed together in a volume that is ≤15%, of the culture volume, ≤10% of the culture volume, ≤5% of the culture volume. Thus, the vectors for rAAV production and transfection reagent are mixed together in a volume that is ≤15%, ≤14%, ≤13%, ≤12%, ≤11%, ≤10%, ≤9%, ≤8%, ≤7%, ≤6%, or ≤5%, of the culture volume.

The cationic peptide or HRP may be added to the cell culture to a concentration of is at least 0.001 μg/mL, at least 0.01 μg/ mL, at least 0.1 μg/mL, at least 0.5 μg/mL, at least 1 μg/mL, at least 10 μg/mL, at least 50 μg/mL, at least 100 μg/mL, 1-200 μg/mL, 5-50 μg/mL, 10-150 μg/mL, or 100-200 μg/mL.

As previously noted, the HRP are also manufactured in GMP grade. The phrase “Good Manufacturing Practice” or “GMP” refers to the set of methods, protocols and procedures established by the United States Food and Drug Administration (FDA) producing products intended for human use. The abbreviation “cGMP” specifically designates those protocols and procedures that are currently approved by the FDA (e.g., under 21 Code of Federal Regulations, part 211). The cationic peptide/HRP and transfection reagent of any embodiment is also biodegradable and have limited to no effect on cell viabilities.

The present inventors have found, for the first time, that HRPs can also be useful as a transfection agent for rAAV production in mammalian cells and rBV production in insect cells. The invention will be further described in the following examples, which do not limit the scope of the invention described in the claims.

### EXAMPLES

### Example 1: Generation of Bba41 Capsids

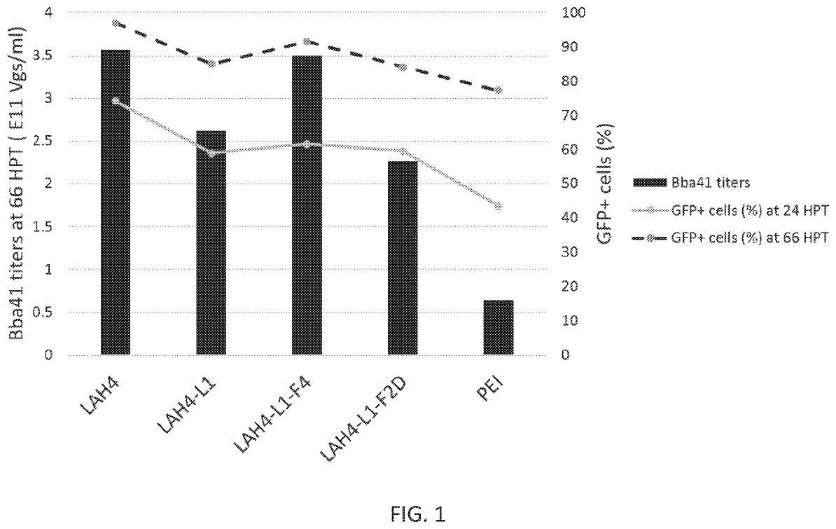

Bba41 capsids were produced in human embryonic kidney 293 (HEK293) cells cultured in Ambr15 minibioreactors (15 mL volume). All of the transfection reagents, cationic peptides, and HRPs were synthetically generated using solid phase peptide synthesis, which can be made under GMP conditions. The HEK293 cells were transfected with plasmids for producing Bba41 capsids and providing an AAV genome vector containing an GFP expression cassette using PEIPRO® and different HRPs including LAH4, LAH4-L1, LAH4-L1-F4, and LAH4-L1-F2D. PEIPRO® is a transfection reagent based of linear polyethylenimine (PEI), which is a cationic polymer. PEIPRO® is optimized for vector production and is considered in the industry to be the gold standard transfection reagent for generating reliable viral vector production and high infectious titer yields. The titers were determined using ddPCR. As shown in FIG. 1, HEK293 cells transfected with the different HRPs produced substantially greater titers of Bba41 capsids (>2.3×10e11 vector genome(s) (vg)/milliliter (mL)) as compared to HEK293 cells transfected with PEIPRO® (˜0.6×10e11 vg/mL). GFP intensity was measured by attune flow cytometer. FIG. 1 also shows that a greater percentage of the HEK293 cells exhibited GFP expression when transfected with the different HRPs (˜58% to ˜74%) at 24 hours post transfection as compared to HEK293 cells transfected with PEIPRO® (˜44%). At 66 hours post transfection, the percentage of HRP transfected HEK293 cells exhibiting GFP expression increased (˜83% to 98%) and remained greater than the PEIPRO® transfected HEK293 cells (˜77%).

In shake flasks (30 mL working volume), Bba41 capsids were produced in HEK293 cells transfected with plasmids for producing Bba41 capsids and providing an AAV genome vector containing an GFP expression cassette using different concentrations and different complexation volumes of the LAH4 peptide. Prior to transfection, the plasmids were mixed with 5, 6, 7, 8, 9, 10, 11, 12, and 13 micrograms (μg)/mL concentrations of the LAH4 peptide. The LAH4/plasmids mixtures had volumes that were 0%, 1%, and 10% of the HEK293 cell culture volume. For example, adding a 0% complexation volume into the HEK293 cell culture indicates that the LAH4 peptide and plasmids were directly added to the culture independent of each other. The titers were determined using ddPCR and GFP intensity was measured by attune flow cytometer.

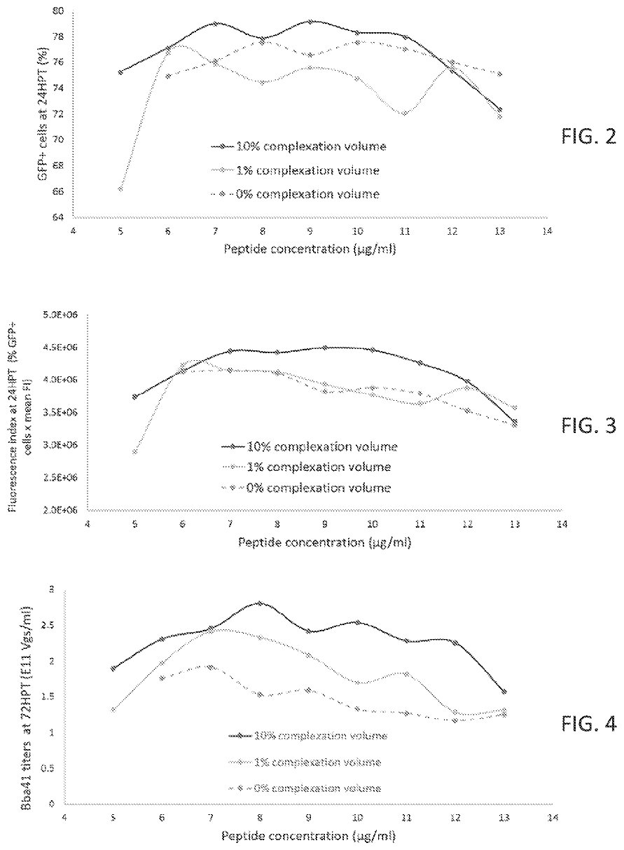

As shown in FIG. 2, 5-13 μg/ml concentrations of LAH4 peptides were able to transfect HEK293 cells at high efficiencies at 0%, 1%, and 10% complexation volumes. FIG. 3 also shows the transfected HEK293 cells exhibiting high GFP fluorescence when transfected with different LAH4 peptide concentrations and complexation volumes.

As shown in FIG. 4, transfections with 5-13 μg/ml concentrations of LAH4 peptides at 0%, 1%, and 10% complexation volumes generated high rAAV titers ranging from ˜1.25×10e11 to ˜2.8×10e11 vg/mL.

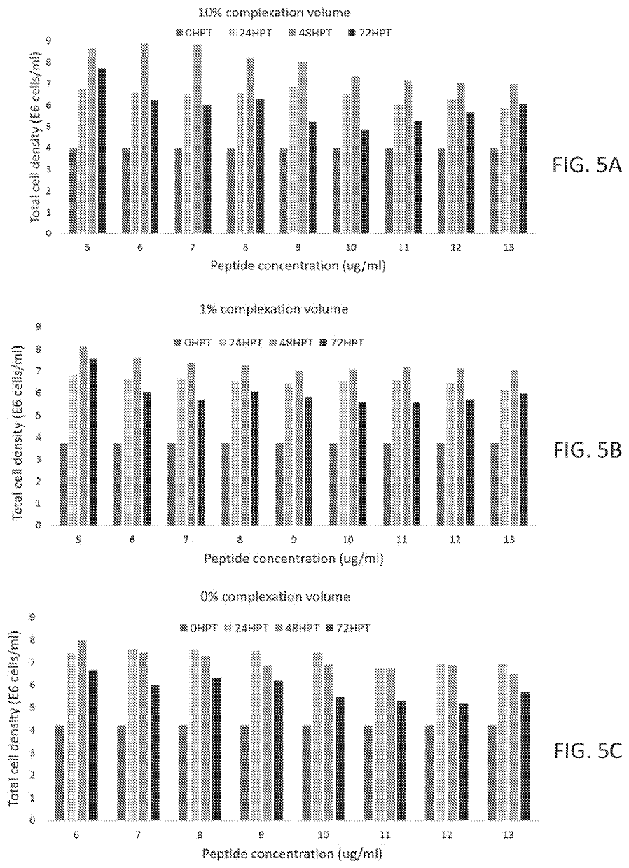

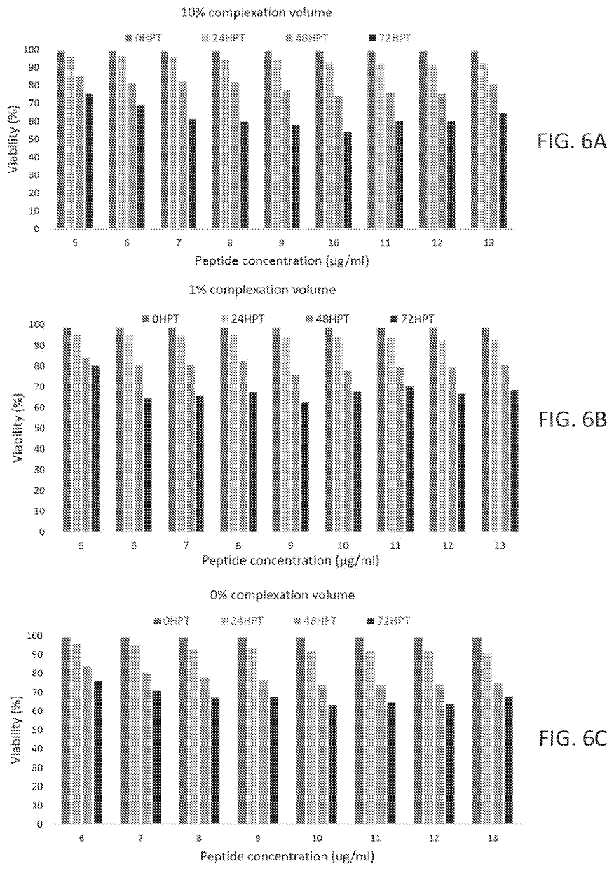

As shown in FIGS. 5A, 5B, and 5C, transfections with 5-13 μg/ml concentrations of LAH4 peptides at 0%, 1%, and 10% complexation volumes had similar cell densities at different time points. FIGS. 6A, 6B, and 6C also show that transfections with 5-13 μg/ml concentrations of LAH4 peptides at 0%, 1%, and 10% complexation volumes had similar viabilities at different time points. The viabilities shown in FIGS. 5A, 5B, and 5C highlight that HRPs at concentrations used for transfections do not induce cytotoxicity.

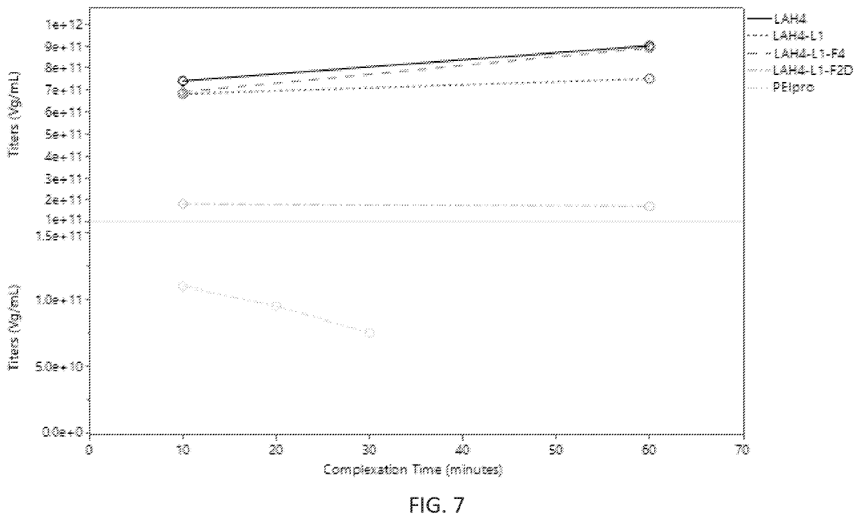

In shake flasks (30 mL working volume), Bba41 capsids were produced in HEK293 cells using a variety of HRPs. The HEK293 cells were transfected with plasmids for producing Bba41 capsids and providing an AAV genome vector containing an GFP expression cassette using different HRPs including LAH4, LAH4-L1, LAH4-L1-F4, and LAH4-L1-F2D. Prior to transfection, the different HRPs and plasmids were mixed together and incubated for 10 and 60 minutes. The titers were determined using ddPCR and GFP intensity was measured by attune flow cytometer. FIG. 7 shows that titers of Bba41 capsids produced in the HEK293 cells prior to transfection, PEIPRO® and the plasmids were mixed together and incubated for 10, 20, and 30 minutes. The titers of Bba41 capsids produced using different HRPs (greater than 6×10e11 vg/mL) were substantially larger than titers of Bba41 capsids produced using PEIPRO® (less than 1.25×10e11 vg/mL). The Bba41 capsid titers also remained high when using the different HRPs at complexation times of 10 and 60 minutes.

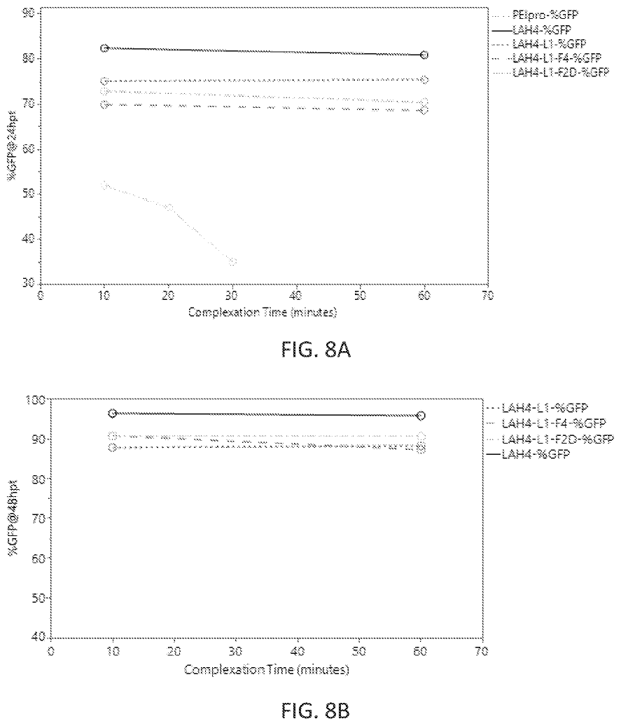

For FIGS. 8A and 8B, the transfection efficiency was determined by the percentage of cells expressing GFP. As shown in FIG. 8A, the transfection efficiency of the different HRPs at 24 hours post transfection was substantially higher than the transfection efficiency of PEIPRO®. The transfection efficiency of the different HRPs at 24 hours post transfection also remained high at complexation times of 10 and 60 minutes. FIG. 8B further shows the transfection efficiency of the different HRPs at 48 hours post transfection remaining high (>85%) at complexation times of 10 and 60 minutes.

FIGS. 1, 2, 3, 4, 5A-5C, 6A-6C, 7, 8A, and 8B highlight that HRPs exhibit better transfection efficiencies in transiently transfected cells such as HEK293 cells as compared to PEI, which is considered to be the gold standard transfection reagent for generating viral vectors. HRPs also generated higher titers of Bba41 capsids as compared to PEI and exhibited enhanced stability when mixed with plasmids and incubated for extended periods of time. PEI when mixed with plasmids does not remain stable over extended periods of time. To this extent, HRPs allow for transfection of greater volumes of cells. For example, a major limitation of current HEK293 cell productions of viral vectors is the inability to scale productions above 500 liters (L).

To test the ability of HRPs to transfect HEK293 cell culture volumes of greater than 500 L, Bba41 capsids are produced in HEK293 cells cultured in different volumes using bioreactors. The bioreactors include cultures with volumes of 100 L, 500 L, 750 L, 1000 L and 2000 L. Prior to transfection, different concentrations of different HRPs are mixed with plasmids for producing Bba41 capsids at different weight ratios. The HRP/plasmids are incubated for different periods of time prior to addition to the cell culture. For each of the different volumes of bioreactor cultures, Bba41 capsids are generated.

### Example 2: Generation of AAV9 Capsids

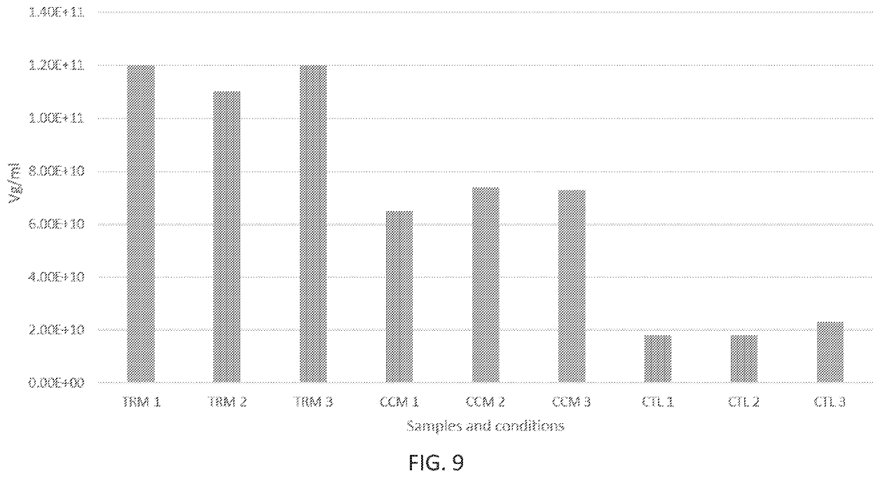

In 1.6 L shake flasks (500 mL working volume), AAV9 capsids were produced in HEK293 cells transfected with plasmids for producing AAV9 capsids and providing an AAV genome vector with a gene of interest using the LAH4 peptide and PEIPRO®. The titers were determined using ddPCR. FIG. 9 shows the AAV9 capsid titers. TRM1, TRM2, and TRM3 are triplicate cultures where the cell culture media was replaced before transfection with the LAH4 peptide. CCM1, CCM2, and CCM3 are triplicate cultures where the cell culture media was not replaced before transfection with the LAH4 peptide. CTL1, CTL2, and CTL3 are triplicate cultures where the HEK293 cells were transfected with PEIPRO®. As shown in FIG. 9, the cultures transfected with the LAH4 peptide (e.g., TRM1-3 and CCM1-3) generated AAV9 capsid titers (6×10e10 vg/mL to ˜1.2×10e11 vg/mL) that were substantially greater than the AAV9 capsid titers (less than 2.2×10e10vg/mL) produced in cultures transfected with PEIPRO® (e.g., CTL1-3).

In Ambr15 minibioreactors (15 mL volume), AAV9 capsids were produced in HEK293 cells transfected with plasmids for producing AAV9 capsids and providing an AAV genome vector containing an GFP expression cassette using different complexation volumes of the LAH4 peptide. Prior to transfection, the plasmids were mixed with a concentration of the LAH4 peptide and incubated for 10 minutes and 60 minutes. The LAH4/plasmids mixtures had volumes that were 1% and 10% of the HEK293 cell culture volume. The titers were determined using ddPCR and GFP intensity was measured by attune flow cytometer.

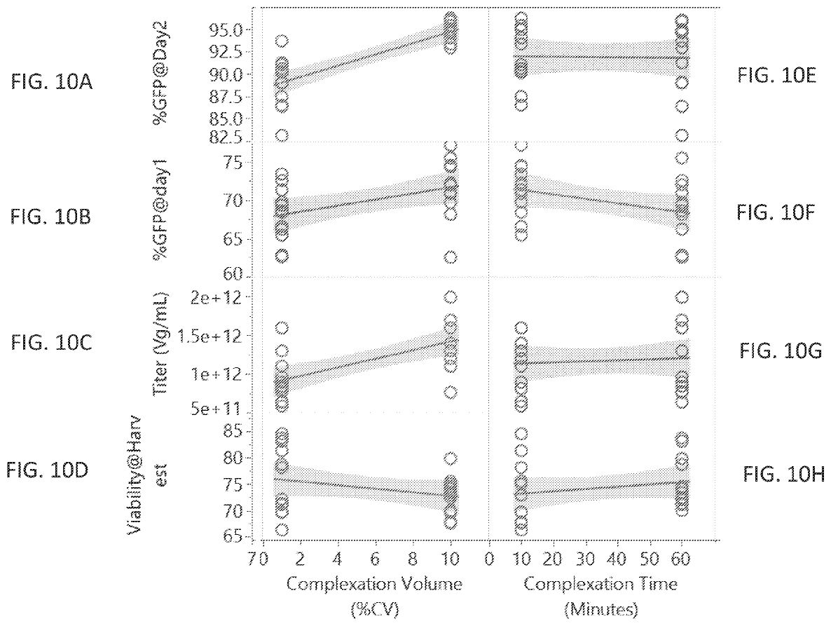

For FIGS. 10A, 10B, 10E, and 10F, the transfection efficiency was determined by the percentage of cells expressing GFP. As shown in FIG. 10A, the HEK293 cells transfected with 1% and 10% complexation volumes of LAH peptide and plasmids resulted in high average transfection efficiencies of greater than 87.5% and 95% at 48 hours. As shown in FIG. 10B, the HEK293 cells transfected with 1% and 10% complexation volumes of LAH4 peptide and plasmids resulted in high average transfection efficiencies of greater than ˜67.5% and greater than 70% at 24 hours. For 10 and 60 minute complexation times, FIG. 10E shows the HEK293 cells transfected with LAH4 peptide/plasmids mixture resulted in a high average transfection efficiency of ˜90% at 48 hours and FIG. 10F shows the HEK293 cells transfected with LAH4 peptide/plasmids mixture resulted in high average transfection efficiencies of ˜70% and ˜67.5% at 24 hours.

As shown in FIG. 10C, the HEK293 cells transfected with 1% and 10% complexation volumes of LAH4 peptide and plasmids produced high titers of AAV9 capsids ranging from ˜5×10e11 to ˜2×10e12 vg/mL. For 10 and 60 minute complexation times, FIG. 10G shows the HEK293 cells transfected with LAH4 peptide/plasmids mixture generating high AAV9 capsid titers with an average titer ranging at ˜1×10e12 vg/mL for both complexation times.

As shown in FIG. 10D, the average viability of the HEK293 cells transfected with 1% and 10% complexation volumes of LAH4 peptide and plasmids was high with greater than 75% and —72.5% cell viabilities. For 10 and 60 minute complexation times, FIG. 10H also shows the average viability of the HEK293 cells transfected with the LAH4 peptide and plasmids mixtures being high with —72.5% and greater 75% cell viabilities. The viabilities shown in FIGS. 10D and 10H highlight that HRPs at concentrations used for transfections do not induce cytotoxicity.

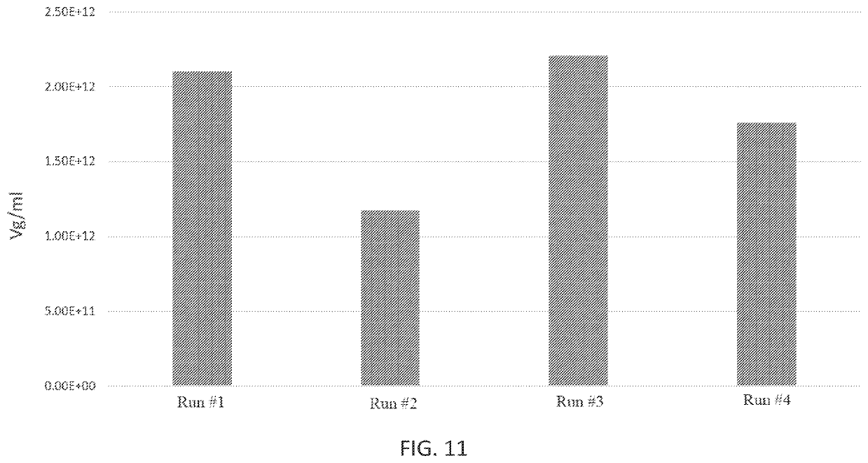

Additional 3L bioreactor productions of the AAV9 capsids were prepared using the LAH4 peptide as the transfection reagent. HEK293 cells were transfected with plasmids for producing AAV9 capsids and providing an AAV genome vector with a gene of interest using the LAH4 peptide. The titers were determined using ddPCR. As shown in FIG. 11, the titers of the additional production generated high AAV9 capsid titers ranging from greater than 1×10e12 vg/mL to ˜2.2×10e12 vg/mL.

FIGS. 9, 10A-10H, and 11 highlight that HRPs exhibit high transfection efficiencies and generated high titers of AAV9 capsids. HRPs also generated higher titers of AAV9 capsids and exhibited enhanced stability when mixed with plasmids and incubated for extended periods of time. PEI when mixed with plasmids does not remain stable over extended periods of time. To this extent, HRPs allow for transfection of greater volumes of cells. For example, a major limitation of current HEK293 productions of viral vectors is the inability to scale productions above 500 L.

Additional HRPs were constructed. Different elements were added to the LAH4 and LAH4-L1-L4 peptides.

For example, the protein transduction domain 4 (PDT4) for enhancing cell binding and penetrating was added to the LAH4 and LAH4-L1-L4 peptides (His-PTD4-LAH4 and SEQ ID NO: 93; PTD4-LAH4 and SEQ ID NO: 94; His-PTD4-LAH4-L1-F4 and SEQ ID NO: 97; PTD4-LAH4-L1-F4 and SEQ ID NO: 98).

Histidine residues for enhanced endosomal escape was added to the LAH4 and LAH4-L1-L4 peptides (His-PTD4-LAH4 and SEQ ID NO: 93; His-Penetratin-LAH4 and SEQ ID NO: 95; His-PTD4-LAH4-L1-F4 and SEQ ID NO: 97; His-Penetratin-LAH4-L1-F4 and SEQ ID NO: 99; His-Penetratin-LAH4-2 and SEQ ID NO: 121; His-Penetratin-LAH4-L1-F4-2 and SEQ ID NO: 123).

A tryptophan residue at the N terminius for stock solution quantitation was added to the LAH4 and LAH4-L1-L4 peptides (LAH4(W) and SEQ ID NO: 101; LAH4-L1-F4(W) and SEQ ID NO: 102).

Penetratin for enhanced cell and DNA binding as well as cell penetrating was added to the LAH4 and LAH4-L1-L4 peptides (His-Penetratin-LAH4 and SEQ ID NO: 95; Penetratin-LAH4 and SEQ ID NO: 96; His-Penetratin-LAH4-L1-F4 and SEQ ID NO: 99; Penetratin-LAH4-L1-F4 and SEQ ID NO: 100; His-Penetratin-LAH4-2 and SEQ ID NO: 121; Penetratin-LAH4-2 and SEQ ID NO: 122; His-Penetratin-LAH4-L1-F4-2 and SEQ ID NO: 123; Penetratin-LAH4-L1-F4-2 and SEQ ID NO: 124).

The simian virus 40 (SV40) nuclear localization signal (NLS) for enhanced nuclear delivery of plasmids was added to the LAH4 and LAH4-L1-L4 peptides (SV40 T NLS-spacer-LAH4 and SEQ ID NO: 103; SV40 T NLS-spacer-LAH4-L1-F4 and SEQ ID NO: 104).

The nucleoplasmin NLS for enhanced nuclear delivery of plasmids was added to the LAH4 and LAH4-L1-L4 peptides (nucleoplasmin NLS-spacer-LAH4 and SEQ ID NO: 105; nucleoplasmin NLS-spacer-LAH4-L1-F4 and SEQ ID NO: 106).

AAV2 VP1-2 BR3 for enhanced delivery of plasmids was added to the LAH4 and LAH4-L1-L4 peptides (AAV2 VP1-2 BR3-spacer-LAH4 and SEQ ID NO: 107; AAV2 VP1-2 BR3-spacer-LAH4-L1-F4 and SEQ ID NO: 108).

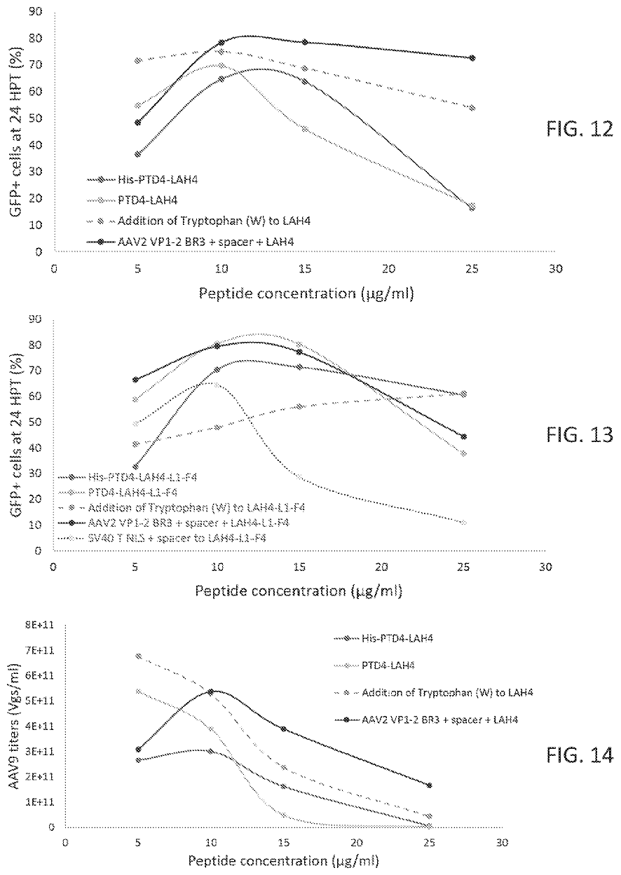

These peptides were chemically synthesized and transfected HEK293 cells at varying concentrations with plasmids containing GFP and various therapeutic transgenes. Particularly, His-PTD4-LAH4, PTD4-LAH4, LAH4(W), AAV2 VP1-2 BR3-spacer-LAH4, His-PTD4-LAH4-L1-F4, PTD4-LAH4-L1-F4, LAH4-L1-F4(W), SV40 T NLS-spacer-LAH4-L1-F4, and AAV2 VP1-2 BR3-spacer-LAH4-L1-F4 were used to transfect HEK293 cells in wells (0.5 mL) of a 96 deep well plate with plasmids for producing rAAV and providing an AAV genome vector containing an GFP expression cassette using the different HRPs at different concentrations. At 24 hours post transfection, the transfection efficiency was measured by identify cells expressing GFP protein. Using 4′,6-diamidino-2-phenylindole (DAPI) staining, the percentage of cells with intact plasma membrane (DAPI negative) in the population of cells to reflect the cytotoxicity of the modified peptide were monitored. Using flow cytometry to gate on cells expressing the GFP protein, the transfection efficiency was expressed as the percentage of positive cells expressing GFP in the population of cells with intact plasma membrane. As shown in FIGS. 12 and 13, the different HRPs were transfect up to ˜80% of the HEK293 cells. Cell viability measurements showed that the different HRPs had limited to no toxicity effects. Accordingly, the different peptides exhibited transfection efficiencies close or equivalent to the LAH4 and LAH4-L1-F4 peptides

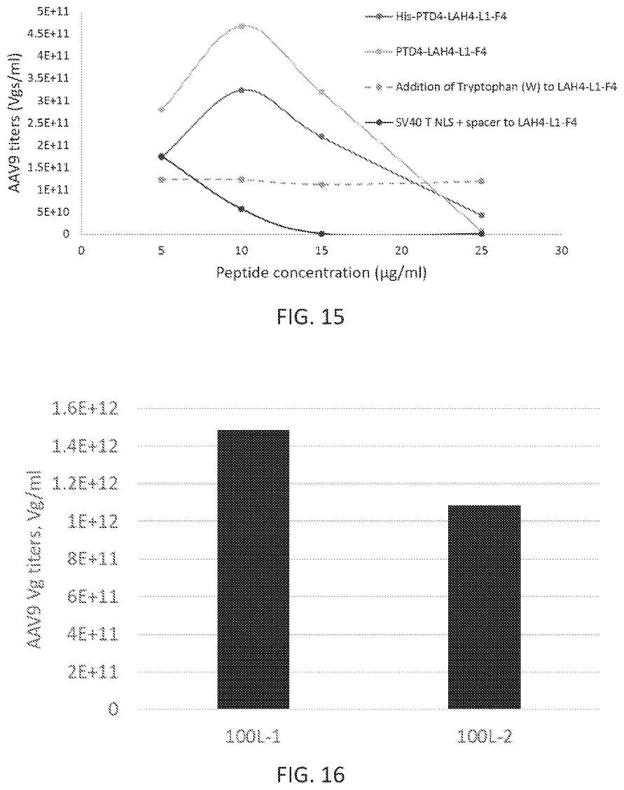

At a predetermined time, AAV capsids were isolated from the cultures. The AAV9 capsid titers were quantified using ddPCR and primers for the vector genomes. As shown in FIGS. 14 and 15, the HEK293 cells transfected with the different HRPs generated titers at the 10e11 vg/mL scale.

### Example 3: Transfection Efficiencies of Histidine Rich Peptides

### Introduction

The goal of the study is to fill the current gap in the knowledge about two properties of LAH4 peptide enabling its use as transfection reagent for large scale transient transfection in biotechnological industry. Two properties are: 1) stability of DNA:LAH4 complexes before transfection; 2) natural clearance of LAH4 peptide post transfection by mammalian cells.

The LAH4 peptide was allowed to incubate with the plasmids for more than 1 hour for large scale (more than or equal 100 L) transient transfections since this incubation time allowed for improved stability of the transfection complexes such that the processes were robust and reproducible. It was discovered that LAH4: DNA complexes are stable at least for 1 hour. In a follow up study, the stability of LAH:DNA complexes are evaluated for up to 1 week when stored at 2-8° C. and/or room temperature and with or without exposure to ambient light.

### Results

Clearance of the ancillary materials such as transfection reagent during bioprocessing is emphasized. We hypothesized that LAH4 is largely degraded by cellular proteases after transfection process is completed. To demonstrate the biodegradable nature of LAH4, we developed a liquid chromatography—mass spectrometry (LC/MS) method to detect LAH4 peptide in the complex matrix of cell lysate. Different concentrations of the LAH4 peptide were added to cell culture volumes of 3 L and 100 L (i.e., bioreactors). At 4 hours post transfection, the LAH4 peptide concentration was reduced by ˜45% to ˜85% relative to initial LAH4 peptide concentration. At 24 hours post transfection, the LAH4 peptide concentration was reduced by ˜83% to ˜97% relative to initial LAH4 peptide concentration. At 48 hours post transfection, the LAH4 peptide concentration was reduced by ˜93% to ˜99% relative to initial LAH4 peptide concentration. At 72 hours post transfection, the LAH4 peptide concentration was reduced by ˜96% to ˜99% relative to initial LAH4 concentration. The data shows that more than 95% clearance of the LAH4 peptide by 72 hours post transfection relative to the initial LAH4 peptide concentration.

### Material and Methods

Cell culturing: Suspension HEK293 cells are cultured according to the manufacturer's instructions in the provided cell culture media. For small scale transfection study HEK293 cells are seeded in the range of at a predetermined cell density in 125 ml Erlenmeyer shake flasks on the day of transfection.

Transfection: LAH4 solution from the same batch is kept at −20° C. in single use aliquots. LAH4 is thawed at RT and mixed with plasmid DNA containing GFP reporter gene. DNA:LAH4 complexes are kept in closed caps tubes to prevent evaporation at either room temperature or 2-8° C. for indicated periods of time before transfection.

Stability of LAH4:DNA complexes: Biophysical characterization of LAH4:DNA complexes is performed. Methods can include size and charge measurements.

Transfection efficiency: Transfection efficiency is measured 24 and 48 hours post transfection using a flow cytometer and expressed as % GFP+cells in the population of single cells with intact plasma membrane.

LAH4 detection in cells: Samples are collected at different time points after transfection and analyzed using LC/MS assay.

### Example 4: Generation of AAV9 Capsids in HEK293 Cells at Large Scale

To test the ability of HRPs to transfect HEK293 cell culture volumes of greater than 500 L, AAV9 capsids are produced in HEK293 cells cultured in different volumes using bioreactors. The bioreactors include cultures with volumes of 100 L, 500 L, 750 L, 1000 L and 2000 L. Prior to transfection, different concentrations of different HRPs are mixed with plasmids for producing AAV9 capsids at different weight ratios. The HRP/plasmids are incubated for different periods of time prior to addition to the cell culture. For each of the different volumes of bioreactor cultures, AAV9 capsids are generated. In different examples, the LAH4 peptide was used to transfect HEK293 cells in suspension in two separate 100 L bioreactor productions. Particularly as shown in FIG. 16, two 100 L productions generated>1.4×10e12 vg/mL and>1×10e12 vg/mL titers. Thus, the present invention allows for large scale production of rAAV using a plasmid/HEK293 system. Accordingly, the generated titers are robust and economically viable such that plasmid/HEK293 systems are now a viable large scale rAAV production system.

### Example 5: Generation of rBV and AAV5 Capsids in Sf9 Cells

As previously highlighted, rAAV can be produced in insect cells (e.g., Sf9 cells) by infecting the cells with recombinant baculovirus (rBV) having nucleotide sequences encoding the capsid and Rep proteins and/or providing an AAV genome vector having an expression cassette. To generate the rBVs, the insect cells such as 519 cells are transfected with recombinant baculovirus genomes having the nucleotide sequences encoding the capsid and Rep proteins and/or providing AAV genome vector having an expression cassette. To generate recombinant baculovirus genomes (i.e., bacmids), different vector systems exist (e.g., Bac-to-Bac™ Baculovirus Expression System, ThermoFisher Scientific) that are capable of inserting the heterologous nucleotide sequences into baculovirus genomes.

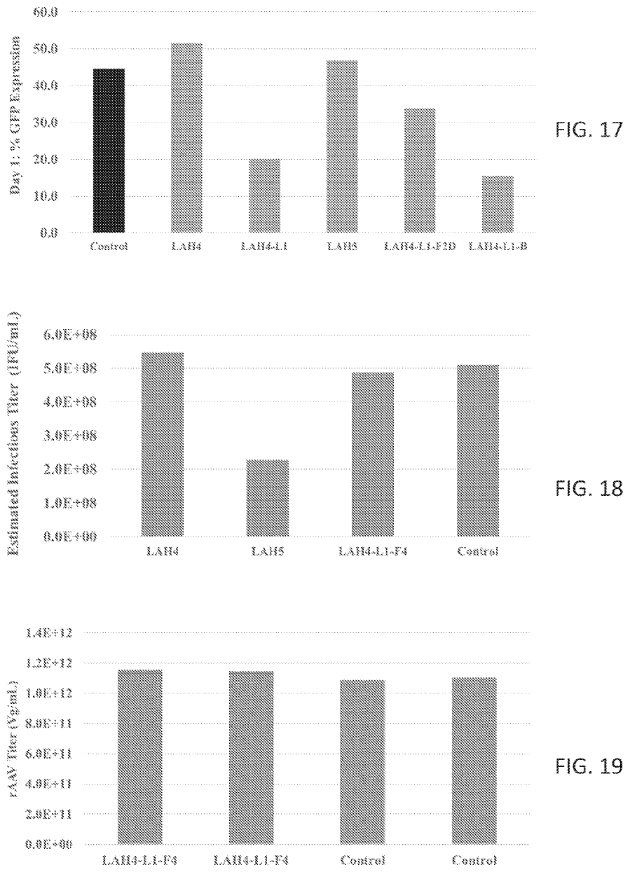

Different HRPs such as LAH4, LAH4-L1, LAH5, LAH4-L1-F2D and a control transfection reagent (CELLFECTIN®, Thermo Fisher) were used to transfect a plasmid with a GFP expression cassette in Sf9 cells. As shown in FIG. 17, 15% to greater than 50% of Sf9 cells transfected with the different HRPs expressed GFP. The transfection efficiencies of the different HRPs were comparable with the transfection efficiency of the control transfection reagent.

AAV5 capsids were also produced in Sf9 cells. To produce the AAV5 capsids, Sf9 cells were transfected with bacmids having nucleotide sequences for generating AAV5 capsids or providing an AAV genome vector using the control transfection reagent and different HRPs. FIG. 18 shows the rBV titers generated from the transfections. In culture, the rBVs generated from the initial bacmid transfection subsequently infect other Sf9 cells that in turn generated more rBVs. As shown in FIG. 18, the rBV titers from LAH4, LAH5, and LAH4-L1-F4 transfected Sf9 cells were comparable to the rBV titers from Sf9 cells transfected with the control reagent.

The different rBVs were collected and used to further infect naive Sf9 cells to generate AAV5 capsids, where the rBVs have nucleotide sequences for generating AAV5 capsids. The titers were determined using ddPCR. As shown in FIG. 19, the rBVs produced from and LAH4-L1-F4 transfected 519 cells generated AAV5 capsid titers comparable to rBVs produced in 519 cells transfected with the control reagent. The AAV5 capsids produced using LAH4-L1-F4 had concentrations of the VP1 capsid protein and ratios of capsid to vector genomes that were the same as AAV5 capsids produced using the control transfection reagent.

To generate large scale productions of AAV5 capsids, different volumes of Sf9 cells are transfected with different HRPs and bacmids for generating AAV5 capsids. The rBVs generated from the transfection infect cultures with volumes of 100 L, 500 L, 750 L, 1000 L and 2000 L to generate AAV5 capsids.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1.-57. (canceled)

58. A method for preparing recombinant adeno-associated virus (rAAV), the method comprising the steps of:
co-transfecting cells with one or more vectors for rAAV production using a transfection reagent, wherein the transfection reagent comprises a histidine rich peptide (HRP);
culturing the transfected cells to generate rAAV; and
recovering the rAAV.

59. A method of preparing recombinant baculovirus (rBV), the method comprising the steps of:
transfecting cells with recombinant bacmid(s) having a baculovirus genome and a heterologous nucleotide sequence using a transfection reagent, wherein the transfection reagent comprises a histidine rich peptide (HRP);
culturing the transfected cells to generate rBV; and
optionally, recovering the rBV.

60. The method according to claim 58, wherein the one or more vectors for rAAV production and transfection reagent are added to cells in a volume that is less than 10% of a volume in which the cells are in culture.

61. The method as in claim 58, wherein the HRP comprises 19 or more amino acids.

62. The method as in claim 59, wherein the HRP comprises 19 or more amino acids.

63. The method as in claim 58, wherein the HRP is prepared according to good manufacturing practices.

64. The method as in claim 58, wherein the HRP is biodegradable.

65. The method as in claim 59, wherein the HRP is prepared according to good manufacturing practices.

66. The method as in claim 59, wherein the HRP is biodegradable.

67. The method as in claim 58, wherein the cells are HEK293, HeLa, CHO, NSO, SP2/0, PER.C6, Vero, RD, BHK, HT 1080, A549, Cos-7, ARPE-19, or MRC-5 cells.

68. The method claim 59, wherein the cells are insect cells derived from Spodoptera frugiperda, Aedes albopictus, Bombyxmori, Trichophusia ni, Ascalapha odorata, Drosphila, Anophele, Culex, or Aedes.

69. The method as in claim 59, wherein the cells are Sf9, High Five, Se301, SeIZD2109, SeUCR1, Sf900+, Sf21, BTI-TN-5B1-4, MG-1, Tn368, HzAm1, BM-N, Ha2302, Hz2E5 or Ao38 cells.

70. The method according to claim 58, wherein the bioreactor has a volume of at least 500 L.

71. The method according to claim 59, wherein the bioreactor has a volume of at least 500 L.

72. The method of claim 58, wherein the one or more vectors for rAAV production and HRP are mixed together prior to the co-transfecting step.

73. The method according to claim 59, wherein the recombinant bacmids and HRP are mixed together prior to the transfecting step.

74. The method as in claim 58, wherein the HRP comprises an amino acid sequence that is at least 85% identical to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124;
culturing the transfected cells to generate rAAV; and
recovering the rAAV.

75. The method as in claim 59, where the cells have at least a portion of a baculovirus genome and the HRP comprises an amino acid sequence that is at least 85% identical to any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124;
culturing the transfected cells to generate rBV, where the heterologous nucleotide sequence and the at least a portion of a baculovirus genome combine to form a baculovirus genome capable of generating rBV; and
optionally, recovering the rBV.

76. An HRP comprising an amino acid sequence that is at least 85% identical to any one of SEQ ID NO: 93, 94, 95, 96, 97, 98, 99, 100, 101, 102, 103, 104, 105, 106, 107, 108, 121, 122, 123, or 124.

77. The HRP as in claim 76, wherein the HRP comprises an amino acid sequence of SEQ ID NO: 93, 94, 95, 96, 97, 98, 99, 100, 101, 102, 103, 104, 105, 106, 107, 108, 121, 122, 123, or 124.


SEQUENCE LISTING (124 sequences)

SEQ ID NO: 1 - protein, 26 aa, Artificial Sequence
  Synthetic LAH4 peptide
KKALLALALHHLAHLALHLALALKKA

SEQ ID NO: 2 - protein, 26 aa, Artificial Sequence
  Synthetic LAH4-L1 peptide
KKALLAHALHLLALLALHLAHALKKA

SEQ ID NO: 3 - protein, 26 aa, Artificial Sequence
  Synthetic LAH5 peptide
KKALLALALHHLAHLAHHLALALKKA

SEQ ID NO: 4 - protein, 26 aa, Artificial Sequence
  Synthetic LAH4-L1-F4 peptide
KKALLAHFFHLLALLALHFFHALKKA

SEQ ID NO: 5 - protein, 26 aa, Artificial Sequence
  Synthetic LAH4-L1-F2D peptide
KKALLAHFLHLLALLALHLFHALKKA

SEQ ID NO: 6 - protein, 26 aa, Artificial Sequence
  Synthetic LAH4 INV-AL peptide
KKALLALALHHALHLALHLALALKKA

SEQ ID NO: 7 - protein, 25 aa, Artificial Sequence
  Synthetic LAH4-L1-? peptide
KKALAHALHLLALALLHLAHALAKK

SEQ ID NO: 8 - protein, 24 aa, Artificial Sequence
  Synthetic LAK4 peptide
KKLAKALAKALAKALKLALALAKK

SEQ ID NO: 9 - protein, 23 aa, Artificial Sequence
  Synthetic LAH1 peptide
KKLALALALALHALALALALKKA

SEQ ID NO: 10 - protein, 23 aa, Artificial Sequence
  Synthetic LAH2 peptide
KKLAHLALALALGLALAHLAKKA

SEQ ID NO: 11 - protein, 24 aa, Artificial Sequence
  Synthetic LAH3 peptide
KKALALGLHLAHLALHLALALKKA

SEQ ID NO: 12 - protein, 26 aa, Artificial Sequence
  Synthetic LAH4-P15 peptide
KKALLALALHHLAHPALHLALALKKA

SEQ ID NO: 13 - protein, 26 aa, Artificial Sequence
  Synthetic D-LAH4 peptide
  misc_feature: D-enantiomer
KKALLALALHHLAHLALHLALALKKA

SEQ ID NO: 14 - protein, 26 aa, Artificial Sequence
  Synthetic LAH4-L1-Opt peptide
KKLALAHALHLALLLALHLAHALKKA

SEQ ID NO: 15 - protein, 26 aa, Artificial Sequence
  Synthetic LAH4X2 peptide
  misc_feature (9)..(9): Xaa is alpha-amino butyric acid
  misc_feature (19)..(19): Xaa is alpha-amino butyric acid
KKALLALAXHHLAHLALHXALALKKA

SEQ ID NO: 16 - protein, 26 aa, Artificial Sequence
  Synthetic LAH5X2 peptide
  misc_feature (9)..(9): Xaa is alpha-amino butyric acid
  misc_feature (19)..(19): Xaa is alpha-amino butyric acid
KKALLALAXHHLAHLAHHXALALKKA

SEQ ID NO: 17 - protein, 26 aa, Artificial Sequence
  Synthetic LAK5X2W2 peptide
  misc_feature (9)..(9): Xaa is alpha-amino butyric acid
  misc_feature (19)..(19): Xaa is alpha-amino butyric acid
KKALLALAXKKWAKLWKKXALALKKA

SEQ ID NO: 18 - protein, 26 aa, Artificial Sequence
  Synthetic peptide
KKALLALALHHLALLAHHLALALKKA

SEQ ID NO: 19 - protein, 26 aa, Artificial Sequence
  Synthetic peptide
KKALLALALHHLALLAHLLALHLKKA

SEQ ID NO: 20 - protein, 29 aa, Artificial Sequence
  Synthetic peptide
KKKGLFHAIHGFIHNGWHGMIHGWYGKKK

SEQ ID NO: 21 - protein, 31 aa, Artificial Sequence
  Synthetic peptide
RRRRRRRRGLFHAIHGFIHNGWHGMIHGWYG

SEQ ID NO: 22 - protein, 31 aa, Artificial Sequence
  Synthetic peptide
CKKKGLFHAIHGFIHNGWHGMIHGWYGKKKC

SEQ ID NO: 23 - protein, 26 aa, Artificial Sequence
  Synthetic peptide
KKKGLFHALLHLLHSLWHLLLHAKKK

SEQ ID NO: 24 - protein, 24 aa, Artificial Sequence
  Synthetic peptide
KKALLAHALHLLALLALHLAHALA

SEQ ID NO: 25 - protein, 26 aa, Artificial Sequence
  Synthetic peptide
RRALLAHALHLLALLALHLAHALRRA

SEQ ID NO: 26 - protein, 26 aa, Artificial Sequence
  Synthetic peptide
KKALLAHALAHLALLALHLALHLKKA

SEQ ID NO: 27 - protein, 26 aa, Artificial Sequence
  Synthetic peptide
KKALLALALHHLALLALHLAHALKKA

SEQ ID NO: 28 - protein, 26 aa, Artificial Sequence
  Synthetic peptide
KKALLHAALAHLLPLAHHLLALLKKA

SEQ ID NO: 29 - protein, 26 aa, Artificial Sequence
  Synthetic peptide
KKALLAKALKLLALLALKLAKALKKA

SEQ ID NO: 30 - protein, 26 aa, Artificial Sequence
  Synthetic peptide
HHALLAHALHLLALLALHLAHALHHA

SEQ ID NO: 31 - protein, 22 aa, Artificial Sequence
  Synthetic peptide
ALLAHALHLLALLALHLAHALA

SEQ ID NO: 32 - protein, 24 aa, Artificial Sequence
  Synthetic peptide
ALLAHALHLLALLALHLAHALKKA

SEQ ID NO: 33 - protein, 24 aa, Artificial Sequence
  Synthetic peptide
ALLHAALAHLLALAHHLLALLKKA

SEQ ID NO: 34 - protein, 26 aa, Artificial Sequence
  Synthetic peptide
KKALLAAALAALLALAHHLLALLKKA

SEQ ID NO: 35 - protein, 39 aa, Artificial Sequence
  Synthetic peptide
GIHKQKEKSRLQGGVLVNEILNHMKRATQIPSYKKLIMY

SEQ ID NO: 36 - protein, 27 aa, Artificial Sequence
  Synthetic peptide
KKKKALLHLHLLALHLHLLALLALKKK

SEQ ID NO: 37 - protein, 18 aa, Artificial Sequence
  Synthetic peptide
KWKLFKKIGAVLKVLTTG

SEQ ID NO: 38 - protein, 14 aa, Artificial Sequence
  Synthetic peptide
CLLKKLLKKLLKKC

SEQ ID NO: 39 - protein, 30 aa, Artificial Sequence
  Synthetic peptide
WEAKLAKALAKALAKHLAKALAKALKACEA

SEQ ID NO: 40 - protein, 12 aa, Artificial Sequence
  Synthetic peptide
YGRKKRRQRRRC

SEQ ID NO: 41 - protein, 17 aa, Artificial Sequence
  Synthetic peptide
RQIKIWFQNRRMKWKKC

SEQ ID NO: 42 - protein, 11 aa, Artificial Sequence
  Synthetic peptide
YARAAARQARA

SEQ ID NO: 43 - protein, 17 aa, Artificial Sequence
  Synthetic peptide
HHHHHHYARAAARQARA

SEQ ID NO: 44 - protein, 30 aa, Artificial Sequence
  Synthetic peptide
YGRKKRRQRRRCKWKLFKKIGAVLKVLTTG

SEQ ID NO: 45 - protein, 42 aa, Artificial Sequence
  Synthetic peptide
YGRKKRRQRRRCWEAKLAKALAKALAKHLAKALAKALKACEA

SEQ ID NO: 46 - protein, 41 aa, Artificial Sequence
  Synthetic peptide
YARAAARQARAWEAKLAKALAKALAKHLAKALAKALKACEA

SEQ ID NO: 47 - protein, 39 aa, Artificial Sequence
  Synthetic peptide
RRRRRRRRRWEAKLAKALAKALAKHLAKALAKALKACEA

SEQ ID NO: 48 - protein, 51 aa, Artificial Sequence
  Synthetic peptide
KETWWETWWTEWSQPKKKRKVWEAKLAKALAKALAKHLAKALAKALKACEA

SEQ ID NO: 49 - protein, 37 aa, Artificial Sequence
  Synthetic peptide
LCLRPVGWEAKLAKALAKALAKHLAKALAKALKACEA

SEQ ID NO: 50 - protein, 40 aa, Artificial Sequence
  Synthetic peptide
RRLSYSRRRFWEAKLAKALAKALAKHLAKALAKALKACEA

SEQ ID NO: 51 - protein, 30 aa, Artificial Sequence
  Synthetic peptide
KWKLFKKIGAVLKVLTTGYGRKKRRQRRRC

SEQ ID NO: 52 - protein, 35 aa, Artificial Sequence
  Synthetic peptide
KWKLFKKIGAVLKVLTTGRQIKIWFQNRRMKWKKC

SEQ ID NO: 53 - protein, 30 aa, Artificial Sequence; identical to SEQ ID NO: 51
  Synthetic peptide
  misc_feature: Includes dimers linked via disulfide bridge between cysteine(s)

SEQ ID NO: 54 - protein, 35 aa, Artificial Sequence; identical to SEQ ID NO: 52
  Synthetic peptide
  misc_feature: Includes dimers linked via disulfide bridge between cysteine(s)

SEQ ID NO: 55 - protein, 36 aa, Artificial Sequence
  Synthetic peptide
KFTIVFPHNQKGNWKNVPSNYHYCPYARAAARQARA

SEQ ID NO: 56 - protein, 34 aa, Artificial Sequence
  Synthetic peptide
LIRLWSHLIHIWFQNRRLKWKKKYARAAARQARA

SEQ ID NO: 57 - protein, 31 aa, Artificial Sequence
  Synthetic peptide
GLFEALLELLESLWELLLEAYARAAARQARA

SEQ ID NO: 58 - protein, 29 aa, Artificial Sequence
  Synthetic peptide
KWKLFKKIGAVLKVLTTGYARAAARQARA

SEQ ID NO: 59 - protein, 35 aa, Artificial Sequence
  Synthetic peptide
CCCCCCKWKLFKKIGAVLKVLTTGYARAAARQARA

SEQ ID NO: 60 - protein, 32 aa, Artificial Sequence
  Synthetic peptide
KWKLFKKIGAVLKVLTTGGGSYARAAARQARA

SEQ ID NO: 61 - protein, 36 aa, Artificial Sequence
  Synthetic peptide
KWKLFKKIGAVLKVLTTGGGSGGGSYARAAARQARA

SEQ ID NO: 62 - protein, 41 aa, Artificial Sequence
  Synthetic peptide
KWKLFKKIGAVLKVLTTGGGSGGGSGGGSGYARAAARQARA

SEQ ID NO: 63 - protein, 37 aa, Artificial Sequence
  Synthetic peptide
MHHHHHHKWKLFKKIGAVLKVLTTGYGRKKRRQRRRC

SEQ ID NO: 64 - protein, 35 aa, Artificial Sequence
  Synthetic peptide
HHHHHHKWKLFKKIGAVLKVLTTGYGRKKRRQRRR

SEQ ID NO: 65 - protein, 35 aa, Artificial Sequence
  Synthetic peptide
HHHHHHKWKLFKKIGAVLKVLTTGYARAAARQARA

SEQ ID NO: 66 - protein, 41 aa, Artificial Sequence
  Synthetic peptide
HHHHHHKWKLFKKIGAVLKVLTTGYARAAARQARACCCCCC

SEQ ID NO: 67 - protein, 33 aa, Artificial Sequence
  Synthetic peptide
HHHHHHKWKLFKKIGAVLKVLTTGRRRRRRRRR

SEQ ID NO: 68 - protein, 50 aa, Artificial Sequence
  Synthetic peptide
HHHHHHKWKLFKKIGAVLKVLTTGGWTLNSAGYLLKINLKALAALAKKIL

SEQ ID NO: 69 - protein, 43 aa, Artificial Sequence
  Synthetic peptide
HHHHHHKKALLALALHHLAHLALHLALALKKAYARAAARQARA

SEQ ID NO: 70 - protein, 31 aa, Artificial Sequence
  Synthetic peptide
HHHHHHCLLKKLLKKLLKKCYARAAARQARA

SEQ ID NO: 71 - protein, 32 aa, Artificial Sequence
  Synthetic peptide
HHHKWKLFKKIGAVLKVLTTGYARAAARQARA

SEQ ID NO: 72 - protein, 41 aa, Artificial Sequence
  Synthetic peptide
HHHHHHHHHHHHKWKLFKKIGAVLKVLTTGYARAAARQARA

SEQ ID NO: 73 - protein, 36 aa, Artificial Sequence
  Synthetic peptide
HHHAHHHKWKLFKKIGAVLKVLTTGYARAAARQARA

SEQ ID NO: 74 - protein, 38 aa, Artificial Sequence
  Synthetic peptide
HAHHAHHAHKWKLFKKIGAVLKVLTTGYARAAARQARA

SEQ ID NO: 75 - protein, 35 aa, Artificial Sequence
  Synthetic peptide
KWKLFKKIGAVLKVLTTGHHHHHHYARAAARQARA

SEQ ID NO: 76 - protein, 41 aa, Artificial Sequence
  Synthetic peptide
HHHHHHKWKLFKKIGAVLKVLTTGYARAAARQARAHHHHHH

SEQ ID NO: 77 - protein, 48 aa, Artificial Sequence
  Synthetic peptide
HHHHHHYARAAARQARAGGGGSKKALLALALHHLAHLALHLALALKKA

SEQ ID NO: 78 - protein, 42 aa, Artificial Sequence
  Synthetic peptide
YARAAARQARAGGGGSKKALLALALHHLAHLALHLALALKKA

SEQ ID NO: 79 - protein, 55 aa, Artificial Sequence
  Synthetic peptide
HHHHHHRQIKIWFQNRRMKWKKGGGGGGSKKALLALALHHLAHLALHLALALKKA

SEQ ID NO: 80 - protein, 49 aa, Artificial Sequence
  Synthetic peptide
RQIKIWFQNRRMKWKKGGGGGGSKKALLALALHHLAHLALHLALALKKA

SEQ ID NO: 81 - protein, 48 aa, Artificial Sequence
  Synthetic peptide
HHHHHHYARAAARQARAGGGGSKKALLAHFFHLLALLALHFFHALKKA

SEQ ID NO: 82 - protein, 42 aa, Artificial Sequence
  Synthetic peptide
YARAAARQARAGGGGSKKALLAHFFHLLALLALHFFHALKKA

SEQ ID NO: 83 - protein, 55 aa, Artificial Sequence
  Synthetic peptide
HHHHHHRQIKIWFQNRRMKWKKGGGGGGSKKALLAHFFHLLALLALHFFHALKKA

SEQ ID NO: 84 - protein, 49 aa, Artificial Sequence
  Synthetic peptide
RQIKIWFQNRRMKWKKGGGGGGSKKALLAHFFHLLALLALHFFHALKKA

SEQ ID NO: 85 - protein, 27 aa, Artificial Sequence
  Synthetic peptide
KKWALLALALHHLAHLALHLALALKKA

SEQ ID NO: 86 - protein, 27 aa, Artificial Sequence
  Synthetic peptide
KKWALLAHFFHLLALLALHFFHALKKA

SEQ ID NO: 87 - protein, 38 aa, Artificial Sequence
  Synthetic peptide
PKKKRKVGGGGSKKALLALALHHLAHLALHLALALKKA

SEQ ID NO: 88 - protein, 38 aa, Artificial Sequence
  Synthetic peptide
PKKKRKVGGGGSKKALLAHFFHLLALLALHFFHALKKA

SEQ ID NO: 89 - protein, 47 aa, Artificial Sequence
  Synthetic peptide
KRPAATKKAGQAKKKKGGGGSKKALLALALHHLAHLALHLALALKKA

SEQ ID NO: 90 - protein, 47 aa, Artificial Sequence
  Synthetic peptide
KRPAATKKAGQAKKKKGGGGSKKALLAHFFHLLALLALHFFHALKKA

SEQ ID NO: 91 - protein, 39 aa, Artificial Sequence
  Synthetic peptide
PARKRLNGGGGSKKWALLALALHHLAHLALHLALALKKA

SEQ ID NO: 92 - protein, 38 aa, Artificial Sequence
  Synthetic peptide
PARKRLNGGGGSKKALLAHFFHLLALLALHFFHALKKA

SEQ ID NO: 93 - protein, 48 aa, Artificial Sequence; identical to SEQ ID NO: 77
  Synthetic peptides

SEQ ID NO: 94 - protein, 28 aa, Artificial Sequence
  Synthetic peptides
YARAAARQARAGGGGSKKALLALALHHL

SEQ ID NO: 95 - protein, 55 aa, Artificial Sequence; identical to SEQ ID NO: 79
  Synthetic peptides

SEQ ID NO: 96 - protein, 49 aa, Artificial Sequence; identical to SEQ ID NO: 80
  Synthetic peptides

SEQ ID NO: 97 - protein, 48 aa, Artificial Sequence; identical to SEQ ID NO: 81
  Synthetic peptides

SEQ ID NO: 98 - protein, 42 aa, Artificial Sequence; identical to SEQ ID NO: 82
  Synthetic peptides

SEQ ID NO: 99 - protein, 55 aa, Artificial Sequence; identical to SEQ ID NO: 83
  Synthetic peptides

SEQ ID NO: 100 - protein, 49 aa, Artificial Sequence; identical to SEQ ID NO: 84
  Synthetic peptides

SEQ ID NO: 101 - protein, 27 aa, Artificial Sequence
  Synthetic peptides
KKWALLALALHHLAHLALHLALALKKA

SEQ ID NO: 102 - protein, 27 aa, Artificial Sequence
  Synthetic peptides
KKWALLAHFFHLLALLALHFFHALKKA

SEQ ID NO: 103 - protein, 38 aa, Artificial Sequence; identical to SEQ ID NO: 87
  Synthetic peptides

SEQ ID NO: 104 - protein, 38 aa, Artificial Sequence; identical to SEQ ID NO: 88
  Synthetic peptides

SEQ ID NO: 105 - protein, 47 aa, Artificial Sequence; identical to SEQ ID NO: 89
  Synthetic peptides

SEQ ID NO: 106 - protein, 47 aa, Artificial Sequence; identical to SEQ ID NO: 90
  Synthetic peptides

SEQ ID NO: 107 - protein, 39 aa, Artificial Sequence; identical to SEQ ID NO: 91
  Synthetic peptides

SEQ ID NO: 108 - protein, 38 aa, Artificial Sequence; identical to SEQ ID NO: 92
  Synthetic peptides

SEQ ID NO: 109 - protein, 26 aa, Artificial Sequence
  Synthetic peptides
KKALLAHALAHLALLALHLALHLKKA

SEQ ID NO: 110 - protein, 26 aa, Artificial Sequence
  Synthetic peptides
KKALLALALHHLALLALHLAHALKKA

SEQ ID NO: 111 - protein, 26 aa, Artificial Sequence
  Synthetic peptides
KKALLALALHHLALLAHHLALALKKA

SEQ ID NO: 112 - protein, 26 aa, Artificial Sequence
  Synthetic peptides
KKALLHLALLHAALLAHHLALALKKA

SEQ ID NO: 113 - protein, 26 aa, Artificial Sequence
  Synthetic peptides
KKALLHLALLHAALLAHLAALHLKKA

SEQ ID NO: 114 - protein, 26 aa, Artificial Sequence
  Synthetic peptides
KKALLHLALLLAALHAHLAALHLKKA

SEQ ID NO: 115 - protein, 26 aa, Artificial Sequence
  Synthetic peptides
KKALLAHALHLLAALALHLAHLLKKA

SEQ ID NO: 116 - protein, 26 aa, Artificial Sequence
  Synthetic peptides
KKALLLAALHHLAALALHLAHLLKKA

SEQ ID NO: 117 - protein, 26 aa, Artificial Sequence
  Synthetic peptides
KKALLLAALHHLLALAHHLAALLKKA

SEQ ID NO: 118 - protein, 26 aa, Artificial Sequence
  Synthetic peptides
KKALLHAALAHLLALAHHLLALLKKA

SEQ ID NO: 119 - protein, 26 aa, Artificial Sequence
  Synthetic peptides
KKALLHALLAHLAALLHALLAHLKKA

SEQ ID NO: 120 - protein, 26 aa, Artificial Sequence
  Synthetic peptides
KKALLHALLAALLAHLHALLAHLKKA

SEQ ID NO: 121 - protein, 53 aa, Artificial Sequence
  Synthetic peptides
HHHHHHRQIKIWFQNRRMKWKKGGGGSKKALLALALHHLAHLALHLALALKKA

SEQ ID NO: 122 - protein, 47 aa, Artificial Sequence
  Synthetic peptides
RQIKIWFQNRRMKWKKGGGGSKKALLALALHHLAHLALHLALALKKA

SEQ ID NO: 123 - protein, 53 aa, Artificial Sequence
  Synthetic peptides
HHHHHHRQIKIWFQNRRMKWKKGGGGSKKALLAHFFHLLALLALHFFHALKKA

SEQ ID NO: 124 - protein, 47 aa, Artificial Sequence
  Synthetic peptides
RQIKIWFQNRRMKWKKGGGGSKKALLAHFFHLLALLALHFFHALKKA